(19) United States
(12) Patent Application Publication
(10) Pub. No.: US 2010/0233537 A1
(43) Pub. Date: Sep. 16, 2010

(54) Triazine Containing Electrode Materials for Secondary Batteries

(76) Inventors: Peter Nesvadba, Marly (CH); Lucienne Bugnon, Pfeffingen (CH)

Correspondence Address: BASF Performance Products LLC;Patent Department, 540 White Plains Road, P.O. Box 2005, Tarrytown, NY, 10591, US

(21) Appl. No.: 12/225,333

(22) PCT Filed: Mar. 12, 2007
(86) PCT No.: PCT/EP2007/052261

§ 371 Date: Sep. 18, 2008

(30) Foreign Application Priority Data
Mar. 21, 2006 (EP) 06111419.5

(51) Int. Cl. H01M 4/60; C07D 401/14; C07D 487/18

(52) U.S. Cl. 429/212; 544/198; 544/219; 540/472; 544/209

(57) ABSTRACT

The invention relates to a stable secondary battery utilizing as active element the oxidation and reduction cycle of a sterically hindered nitroxide radical, which is bonded to a triazine structural element. Further aspects of the invention are a method for providing such a secondary battery, the use of the respective compounds as active elements in secondary batteries and selected novel nitroxide compounds as such.

The invention relates to a stable secondary battery utilizing as active element the oxidation and reduction cycle of a sterically hindered nitroxide radical, which is bonded to a triazine structural element. Further aspects of the invention are a method for providing such a secondary battery, the use of the respective compounds as active elements in secondary batteries and selected novel nitroxide compounds.

Nitroxide polymers as cathode active materials in organic radical batteries have already been described, for example, in Electrochimica Acta 50, 827 (2004). The preparation of 4-meth-acryloyloxy-2,2,6,6-tetramethylpiperidine, its free radical polymerization and subsequent oxidation of the polymer into the corresponding polymeric nitroxide is described.

The use of various radicals, such as, for example, nitroxide radicals as active component in electrode materials of secondary batteries has been also disclosed in EP 1 128 453. Since low solubility or insolubility of the electrode material in the battery electrolyte is preferable, polymeric or oligomeric nitroxides are of particular interest.

Due to the fast growing market of electronic devices, such as mobile telephones and mobile personal computers (lap-tops), there have been increasing needs in the last years for small and large-capacity secondary batteries with high energy density.

Today the most frequently used secondary battery for such applications is the lithium-ion secondary battery. Such a lithium-ion secondary battery uses a transition-metal oxide containing lithium in the positive electrode (cathode) and carbon in a negative electrode (anode) as active materials, and performs charge and discharge via insertion of Li in and elimination of Li from these active materials.

However, since the lithium-ion secondary battery uses a transition-metal oxide with a large specific gravity, particularly in the positive electrode, it has an undesirable secondary battery capacity per unit weight. There have been, therefore, attempts for developing a large-capacity secondary battery using a lighter electrode material. For example, U.S. Pat. Nos. 4,833 and 2,715,778 have disclosed a secondary battery using an organic compound having a disulfide bond in a positive electrode, which utilizes, as a principle of a secondary battery, an electrochemical oxidation-reduction reaction associated with formation and dissociation of a disulfide bond.

As mentioned above EP 1 128 453 similarly discloses, for example, nitroxide radicals as active components in electrode materials of secondary batteries.

Surprisingly it has now been found that nitroxide radicals chemically linked to 1,3,5 triazines can afford active electrode materials having a charge capacity higher than the one theoretically achievable from the reversible oxidatio/reduction of all nitroxide groups present in the molecule. Indeed, the molecular combinations of 1,3,5 triazines with nitroxides can show an unexpected, surprising synergistic effect. This effect has so far not been recognized or described.

One aspect of the invention is a secondary battery with improved capacity, utilizing an electrode reaction of an active material in the reversible oxidation/reduction cycle in at least one of the positive or negative electrodes, which active material comprises a compound selected from the group consisting of radicals of formulae (Ia) to (Iq), which are chemically bonded directly or via a linking group to a triazine structural element of formula (II)

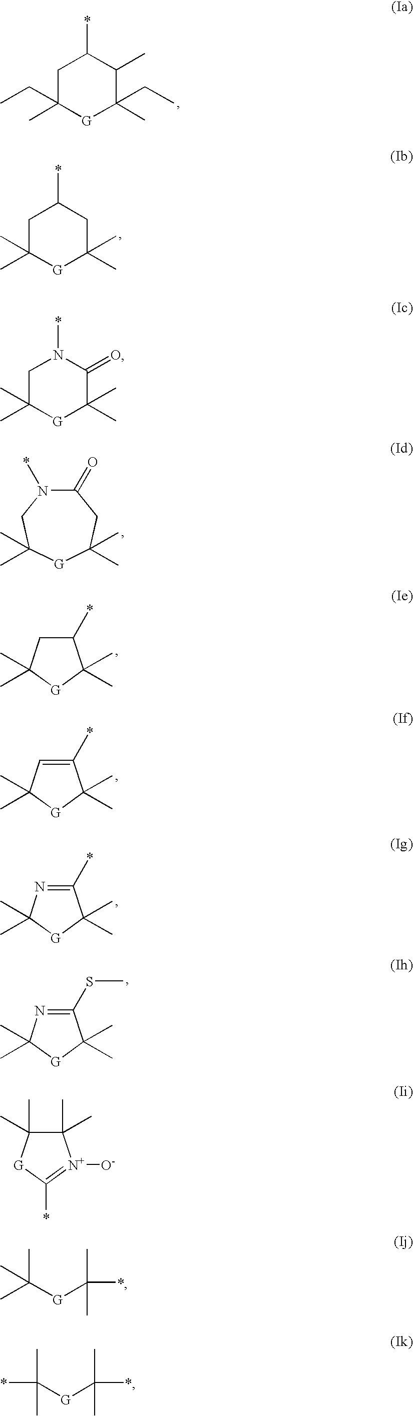

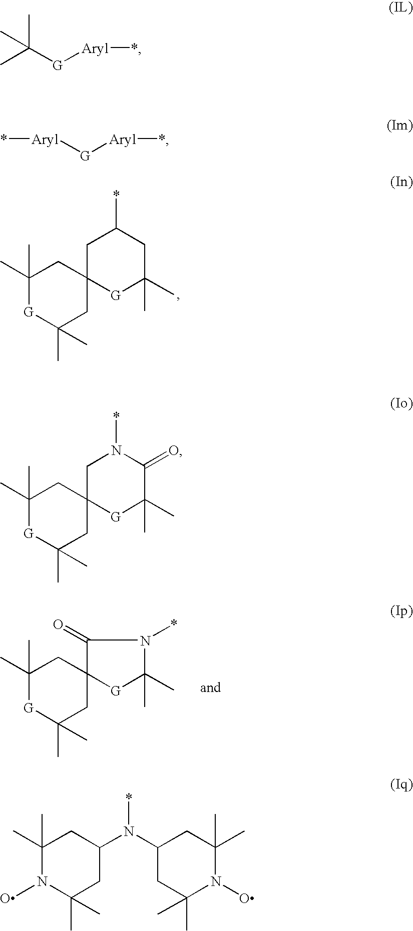

wherein
* is a valence indicating the bond to the triazine structural element;
Aryl is phenyl or naphthyl and

### G is

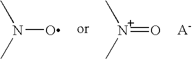

wherein A⁻ is an anion derived from an organic or inorganic acid;

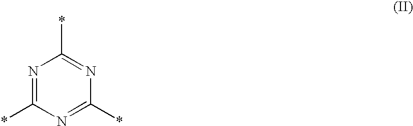

wherein
* is indicating a bond to which at least one radical of formulae (Ia) to (Iq) is attached, either directly or via a linking group;
with the proviso that compound (A) is excluded

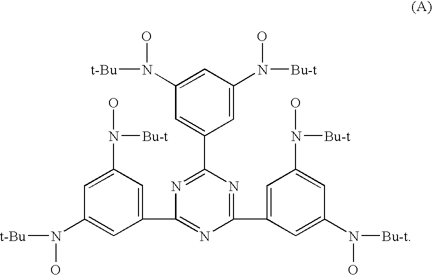

This invention provides a secondary battery using a radical compound as an electrode active material. When the radical compound consists of lighter elements such as carbon, hydrogen and oxygen, it may be expected to provide a secondary battery with a high energy density per weight.

An electrode active material as used herein refers to a material directly contributing to an electrode reaction such as charge and discharge reactions, and plays a main role in a secondary battery system. An active material in this invention may be used as either a positive electrode or negative electrode active material, but it may be more preferably used as a positive electrode active material because it is characterized by a light weight and has a good energy density in comparison with a metal oxide system.

The underlying mechanism of energy storage is the reversible oxidation/reduction of the nitroxide radical according to Scheme 1:

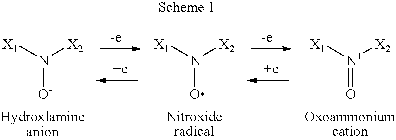

The counter ion of the oxoammonium cation, A⁻ may be, for example, the anion derived from LiPF₆, LiCl0₄, LiBF₄, LiCF₃S0₃, LiN(CF₃SO₂)₂, LiN(C₂F₅S0₂)₂, LiC(CF₃SO₂)₃ and LIC(C₂F_{S}S0₂)₃.

Even though the use of the full redox window (hydroxylamine anion <-> oxoammonium cation) is possible, the currently preferred batteries use the redox pair nitroxide radical <-> oxoammonium cation. Hence, the electrons are exchanged between the oxidized state N⁺═O and reduced state N—O*.

Therefore, preferred is a secondary battery wherein the above electrode reaction is that in the positive electrode.

In this invention, a binder may be used for reinforcing binding between components.

Examples of a binder include polyvinylidene fluoride, a copolymer of vinylidene fluoride and hexafluoropropylene, a copolymer of vinylidene fluoride and tetrafluoroethylene, polytetrafluoroethylene, a copolymer rubber of styrene and butadiene, and resin binders such as polypropylene, polyethylene and polyimide.

According to the invention the active material in at least one of a positive electrode and a negative electrode comprises a radical compound bonded to a triazine structural element, without restrictions to its amount. However, since the capacity as a secondary battery depends on the amount of the radical compound contained in the electrode, the content is desirably 10 to 100% by weight, preferably 20 to 100% and in particular 50 to 100% for achieving adequate effects.

It is also possible to use more than one radical compound as active electrode material. The compound according to the invention may be mixed, for example, with a known active material to function as a complex active material.

When using the instant radical compound in a positive electrode, examples of materials for the negative electrode layer include carbon materials such as graphite and amorphous carbon, lithium metal or a lithium alloy, lithium-ion occluding carbon and conductive polymers. These materials may take an appropriate form such as film, bulk, granulated powder, fiber and flake.

A conductive auxiliary material or ion-conductive auxiliary material may also be added for reducing impedance during forming the electrode layer. Examples of such a material include carbonaceous particles such as graphite, carbon black and acetylene black and conductive polymers such as polyaniline, polypyrrole, polythiophene, polyacetylene and polyacene as conductive auxiliary materials as well as a gel electrolyte and a solid electrolyte as ion-conductive auxiliary material.

A preferred embodiment of the invention is a secondary battery wherein the active material comprises from 10 to 100% by weight of the compound selected from the group consisting of radicals of formulae (Ia) to (Iq), which are chemically bonded to a triazine structural element of formula (II).

For instance, the remainder of the active material, especially in lithium or lithium-ion secondary batteries, contain a lithium transition metal/main group metal composite oxide. Such active material is for example LiMnP0₄, LiCo0₂, LiNi0₂, LiNi₁₋ₓCo_{y}Met_{z}0₂, LiMn_{0.5}Ni_{0.5}0₂, LiMn_{0.3}Co_{0.3}Ni_{0.3}0₂, LiFeP0₄, LiMn₂0₄, LiFe0₂, LiMet_{0.5}Mn_{1.5}0₄, vanadium oxide, or mixtures of any two or more thereof, wherein Met is Al, Mg, Ti, B, Ga. Si, Ni, or Co, and wherein 0<x<0.3, 0<z<0.5, 0<y<0.5. Further suitable active material is a spinel manganese oxide with a formula of Li₁₊ₓMn_{2-z}Met_{Y}0₄₋ₘXₙ, wherein Met is Al, Mg, Ti, B, Ga, Si, Ni, or Co, and X is S or F, and wherein 0<x<0.3, 0<z<0.5, 0<y<0.5, 0<m<0.5 and 0<n<0.5.

A catalyst may also be used for accelerating the electrode reaction. Examples of a catalyst include conductive polymers such as polyaniline, polypyrrole, polythiophene, polyacetylene and polyacene; basic compounds such as pyridine derivatives, pyrrolidone derivatives, benzimidazole derivatives, benzothiazole derivatives and acridine derivatives; and metal-ion complexes.

The concentration of the radical compound in this invention is preferably kept to 10¹⁹ spin/g or more, more preferably 10²¹ spin/g or more. With regard to the capacity of a secondary battery as many spins/g as possible is desirable.

In general, a radical concentration may be expressed as a spin concentration. That is, a spin concentration means the number of unpaired electrons (radicals) per unit weight, which is determined by, for example, the following procedure from an absorption area intensity in an electron spin resonance spectrum (hereinafter, referred to as an “ESR” spectrum). First, a sample to be measured by ESR spectroscopy is pulverized by grinding it in, for example, a mortar, whereby the sample may be ground to a particle size in which skin effect, i.e., a phenomenon that microwave does not penetrate a sample, can be ignored. A given amount of the pulverized sample is filled in a quartz glass capillary with an inner diameter of 2 mm or less, preferably 1 to 0.5 mm, vacuumed to 10-5 mm Hg or less, sealed and subjected to ESR spectroscopy. ESR spectroscopy may be conducted in any commercially available model. A spin concentration may be determined by integrating twice an ESR signal obtained and comparing it to a calibration curve. There are no restrictions to a spectrometer or measuring conditions as long as a spin concentration can be accurately determined. For the stability of a secondary battery, a radical compound is desirably stable. A stable radical as used herein refers to a compound whose radical form has a long life time.

Therefore, preferred is a secondary battery wherein the active material has a spin concentration of at least 10²¹ spins/g.

As outlined in Scheme 1 the underlying mechanism of energy storage is the reversible oxidation/reduction of the nitroxide radical. That means during charging and discharging always two species are present, namely the nitroxide radical and its oxidized or reduced form, depending on whether it is the active material of the positive or negative electrode.

In a preferred embodiment of a secondary battery G is a nitroxide radical

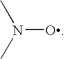

A secondary battery according to this invention has a configuration, for example, as described in EP 1 128 453, where a negative electrode layer and a positive electrode layer are piled via a separator containing an electrolyte. The active material used in the negative electrode layer or the positive electrode layer is a radical compound bonded to a triazine structural element as described above.

In another configuration of a laminated secondary battery a positive electrode collector, a positive electrode layer, a separator containing an electrolyte, a negative electrode layer and a negative electrode collector are piled in sequence. The secondary battery may be a multi-layer laminate as well, a combination of collectors with layers on both sides and a rolled laminate.

The negative electrode collector and the positive electrode collector may be a metal foil or metal plate made of, for example, from nickel, aluminum, copper, gold, silver, an aluminum alloy and stainless steel; a mesh electrode; and a carbon electrode. The collector may be active as a catalyst or an active material may be chemically bound to a collector. A separator made of a porous film or a nonwoven fabric may be used for preventing the above positive electrode from being in contact with the negative electrode.

An electrolyte contained in the separator transfers charged carriers between the electrodes, i.e., the negative electrode and the positive electrode, and generally exhibits an electrolyte-ion conductivity of 10⁻⁵ to 10⁻¹ S/cm at room temperature. An electrolyte used in this invention may be an electrolyte solution prepared by, for example, dissolving an electrolyte salt in a solvent. Examples of such a solvent include organic solvents such as ethylene carbonate, propylene carbonate, dimethyl carbonate, diethyl carbonate, methyl ethyl carbonate, y-butyrolactone, tetrahydrofurane, dioxolane, sulforane, dimethylformamide, dimethylacetamide and N-methyl-2-pyrrolidone. In this invention, these solvents may be used alone or in combination of two or more. Examples of an electrolyte salt include LiPF₆, LiCl0₄, LiBF₄, LiCF₃S0₃, LiN(CF₃SO₂)₂, LiN(C₂F₆S0₂)₂, LiC(CF₃SO₂)₃ and LIC(C₂F_{S}S0₂)₃.

An electrolyte may be solid. Examples of a polymer used in the solid electrolyte include vinylidene fluoride polymers such as polyvinylidene fluoride, a copolymer of vinylidene fluoride and hexafluoropropylene, a copolymer of vinylidene fluoride and ethylene, a copolymer of vinylidene fluoride and monofluoroethylene, a copolymer of vinylidene fluoride and trifluoroethylene, a copolymer of vinylidene fluoride and tetrafluoroethylene and a terpolymer of vinylidene fluoride, hexafluoropropylene and tetrafluoroethylene; acrylonitrile polymers such a copolymer of acrylonitrile and methyl methacrylate, a copolymer of acrylonitrile and methyl acrylate, a copolymer of acrylonitrile and ethyl methacrylate, a copolymer of acrylonitrile and ethyl acrylate, a copolymer of acrylonitrile and methacrylic acid, a copolymer of acrylonitrile and acrylic acid and a copolymer of acrylonitrile and vinyl acetate; polyethylene oxide; a copolymer of ethylene oxide and propylene oxide; and polymers of these acrylates or methacrylates. The polymer may contain an electrolyte solution to form a gel or the polymer may be used alone.

A secondary battery in this invention may have a conventional configuration, where, for example, an electrode laminate or rolled laminate is sealed in, for example, a metal case, a resin case or a laminate film made of a metal foil such as aluminum foil and a synthetic resin film. It may take a shape of, but not limited to, cylindrical, prismatic, coin or sheet.

A secondary battery according to this invention may be prepared by a conventional process. For example, from slurry of an active material in a solvent applied on an electrode laminate. The product is piled with a counter electrode via a separator. Alternatively, the laminate is rolled and placed in a case, which is then filled with an electrolyte solution. A secondary battery may be prepared using the radical compound itself or using a compound which can be converted into the radical compound by a redox reaction, as already described above.

The active material comprises a compound selected from the group consisting of radicals of formulae (Ia) to (Iq), which are chemically bonded directly or via a linking group to a triazine structural element of formula (II).

Examples for linking groups are C₁-C₁₂alkylene, phenylene, amine or alkylamine groups, such as —NH—, —N(C₁-C₁₂)— or —N(C₅-C₆cycloalkyl)- and the —O-atom.

For example the secondary battery contains as active element a compound wherein to the triazine structural element of formula (II) at least two radicals of formulae (Ia) to (Iq) are bonded.

For instance, the secondary battery contains as active element a compound wherein the triazine structural element of formula (II) is part of the repeating unit of an oligomer or polymer.

Preferred radicals, which are bonded to the triazine structural element are of formulae (Ib), Ic), (In), (Ip) or (Iq).

For example the active material comprises a compound of formulae (c1) to (c7)

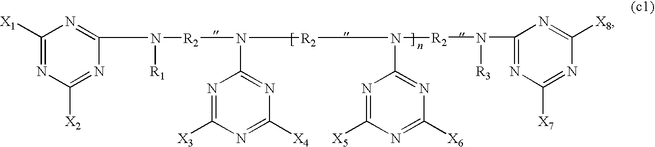

wherein
n is a number from 0-100;
R₁ and R₃ are independently H or C₁-C₁₂alkyl, C₅-C₇cycloalkyl, phenyl, C₁-C₁₂acyl or a group

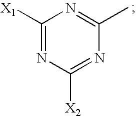

R₂ is C₂-C₁₂alkylene or phenylene;
X₁-X₈ independently of one another are groups of the formulas (c31)-(c45), preferably (c31) or (c32),

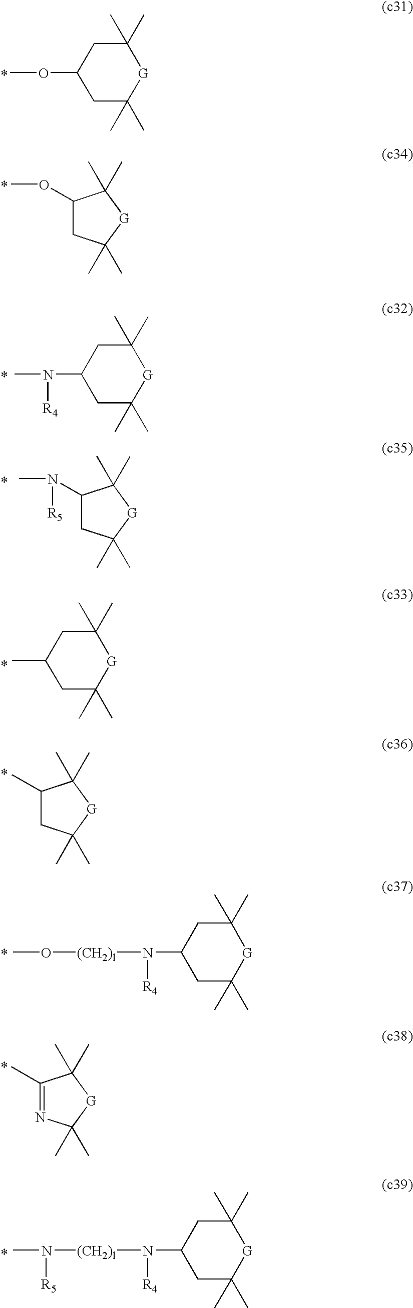

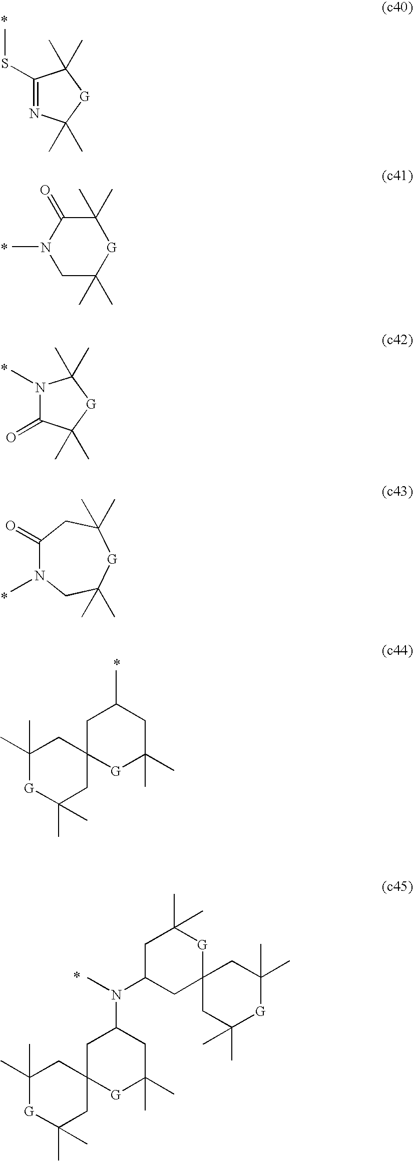

wherein

### I is 2-6;

R₄ and R₅ are hydrogen, C₁-C₁₂alkyl, C₅-C₆cycloalkyl, phenyl, C₁-C₁₂acyl or a group of formula (c33), (c36) or (c44);

additionally one or more of X₁-X₈ are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, NH₂, —NH—NH₂, —SH, C₁-C₁₂alkyl phenyl, H, C₁-C₁₂alkenylamino, C₁-C₁₂alkynylamino, —SCN, —NHNH—C₁-C₁₂Alkyl, —NHNH-Phenyl, —N₃, —CN, C₅-C₆cycloalkyl, C₂-C₁₂alkenyl, C₂-C₁₂alkynyl, wherein the said alkyls are unsubstituted or substituted by OR₄, —NR₄R₅ or SR₄, or the said alkyls comprise at least two carbon atoms and are uninterrupted or interrupted by —O—, —S— or —NR₄— or the said alkyl comprises at least two carbon atoms and is both substituted by OR₄, —NR₄R₅ or SR₄ and interrupted by —O—, —S— or —NR₄—;

with the proviso that at least one of X₁ to X₈ is a group according to the formulas (c31)-(c45); and
G is as defined in claim 1;

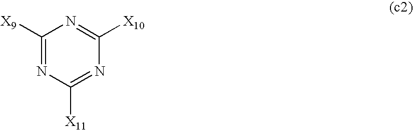

X₉-X₁₁ independently of one another are groups as defined for X₁-X₈;

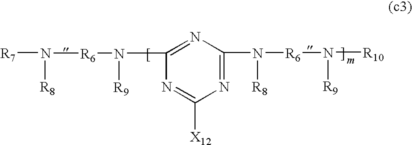

wherein
m is 0-100
R₆ is C₂-C₁₂alkylene, phenylene or a direct bond;
R₇, R₈, R₉ and R₁₀ independently are H or C₁-C₁₂alkyl, C₅-C₆cycloalkyl, phenyl, C₁-C₁₂acyl or the groups (c33) or (c36) or a group

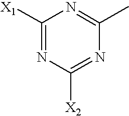

and X₁₂ has the meaning as defined for X₁-X₈;

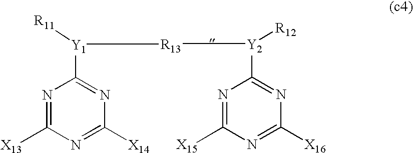

wherein

### Y₁ and Y₂ are N, O or S

R₁₃ is C₂-C₁₂alkylene, phenylene, C₂-C₁₂ diacyl or a direct bond if Y₁ and Y₂ are N or S;
If Y₁ or Y₂ are O or S, then R₁₁ or R₁₂ are an electron pair;
additionally, if Y₁ is N, then Y₂ and R₁₃ together can be direct bond;
If Y₁ and Y₂ are N, then R₁₁ and R₁₂ independently are H or C₁-C₁₂alkyl, C₅-C₆cycloalkyl, C₁-C₁₂acyl, phenyl or a group of formula (c33) or (c36) or a group

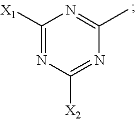

X₁₃-X₁₆ are independently as defined for X₁-X₈;

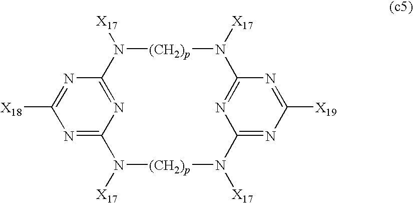

wherein
p is a number 0-18
X₁₇ is independently a group (c33) or (c36); and
X₁₈ and X₁₉ are independently groups as defined for X₁-X₈;

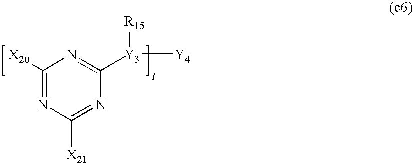

wherein
X₂₀ and X₂₁ are independently groups as defined for X₁-X₈,

### Y₃ is N, O or S;

If Y₃ is O or S, then R₁₅ is an electron pair;
If Y₃ is N, then R₁₅ is H or C₁-C₁₂alkyl, C₅-C₆cycloalkyl, C₁-C₁₂acyl, phenyl or a group of formula (c33) or (c36) or a group

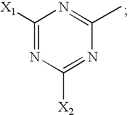

t is 3-6;
Y₄ is a trivalent-, tetravalent-, pentavalent or hexavalent inorganic or organic residue;

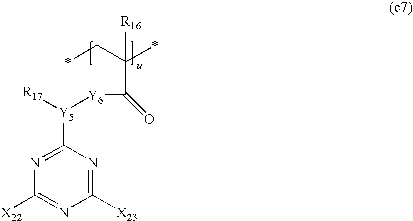

wherein

### R₁₆ is H or CH₃;

### Y₅ is N, O or S;

If Y₅ is O or S, then R₁₇ is an electron pair
If Y₅ is N, then R₁₇ is H or C₁-C₁₂alkyl, C₅-C₆cycloalkyl, C₁-C₁₂acyl, phenyl or a group of formula (c33) or (c36) or a group

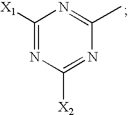

Y₆ is a direct bond, —NH— or —CH₂CH₂—N— or —CH₂CH₂—O—; and
X₂₂ and X₂₃ have the same meaning as defined for X₁-X₈.

Examples of alkyl having up to 12 carbon atoms are methyl, ethyl, propyl, isopropyl, n-butyl, sec-butyl, isobutyl, tert-butyl, 2-ethylbutyl, n-pentyl, isopentyl, 1-methylpentyl, 1,3-dimethyl-butyl, n-hexyl, 1-methylhexyl, n-heptyl, isoheptyl, 1,1,3,3-tetramethylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl,

Examples of alkoxy having up to 12 carbon atoms are methoxy, ethoxy, propoxy, isopropoxy, butoxy, isobutoxy, pentoxy, isopentoxy, hexoxy, heptoxy, octoxy, decyloxy, dodecyloxy,

Examples of C₅-C₆cycloalkyl are cyclopentyl and cyclohexyl, especially cyclohexyl, is preferred.

Examples of acyl containing 8 carbon atoms are formyl, acetyl, propionyl, butyryl, pentanoyl, hexanoyl, heptanoyl, octanoyl, acryloyl, methacryloyl and benzoyl.

Examples of alkylene having up to 12 carbon atoms are methylene, ethylene, propylene, trimethylene, tetramethylene, pentamethylene, 2,2-dimethyltrimethylene, hexamethylene, trimethylhexamethylene, octamethylene and decamethylene.

Examples of halogen are F, Cl, Br and I, especially F and Cl.

Examples of trivalent and tetravalent residues are, for example, the following groups

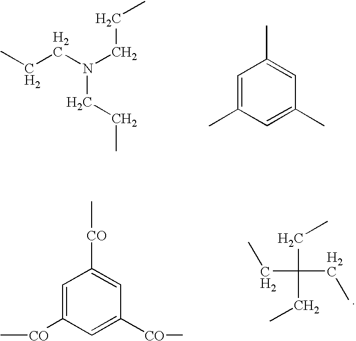

For instance, X₁-X₈ independently of one another are groups of the formulas (c31)-(c45), preferably (c31) or (c32);

additionally one or more of X₁-X₈ are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, —NH₂, —NH—NH₂, —SH, C₁-C₁₂alkyl or phenyl.

For instance, X₁-X₈ for compounds of formula (c1), (c3)-(c7) are as defined above.

Compounds of formula (c2) are preferred.

Preferably the active material comprises a compound of formulae (d1) to (d7)

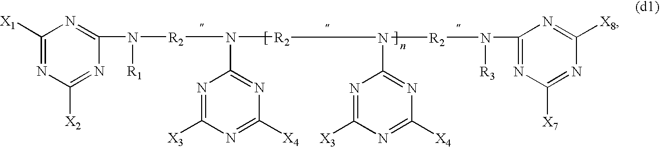

wherein
n is a number from 0-50
R₁ and R₃ are independently H, methyl, formyl, acetyl or a group

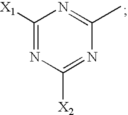

R₂ is C₂-C₆alkylene;
X₁-X₈ independently of one another are groups of the formulas (d31)-(d34), preferably (d31) or (d32)

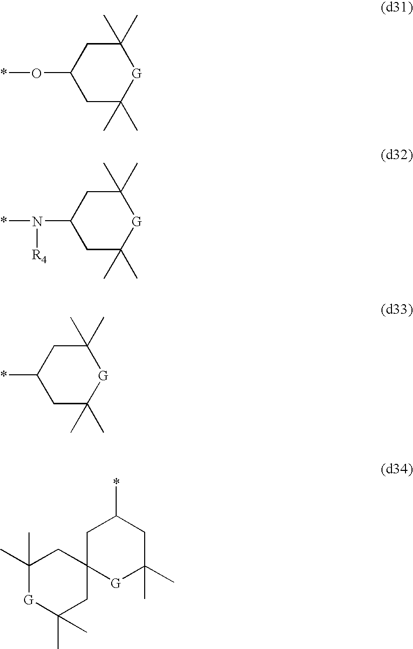

additionally one or more of X₁-X₈ are C₁-C₁₂alkyloxy, C₁-C₁₂alkylamino, C₁-C₁₂dialkylamino, carboxymethyl, halogen, —OH, NH₂, —SH, C₁-C₁₂alkyl H, C₁-C₁₂alkenylamino or C₁-C₁₂alkynylamino;

### G is

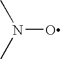

R₄ is hydrogen, C₁-C₄alkyl or a group of formula (d33) or (d34);

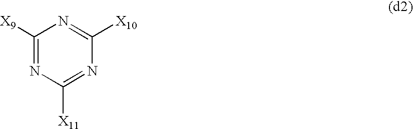

X₉-X₁₁ independently of one another are as defined for X₁-X₈;

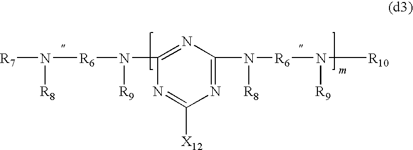

wherein
m is 0-50
R₆ is C₂-C₆alkylene or a direct bond;
R₇, R₈, R₉ and R₁₀ independently are H, methyl, formyl, acetyl or a group

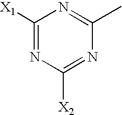

and X₁₂ has the meaning as defined for X₁-X₈;

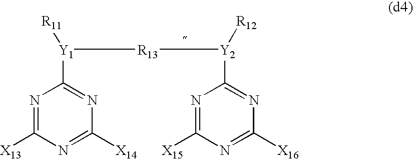

wherein

### Y₁ and Y₂ are N, O or S

R₁₃ is C₂-C₆alkylene or a direct bond if Y₁ and Y₂ are N or S;
If Y₁ or Y₂ are O or S, then R₁₁ or R₁₂ are an electron pair;
If Y₁ and Y₂ are N, then R₁₁ and R₁₂ independently are H, C₁-C₄alkyl or a group of formula (d33) or a group

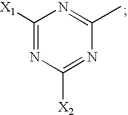

X₁₃-X₁₆ are independently as defined for X₁-X₈;

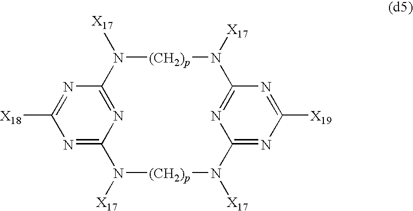

wherein
p is a number 2-6
X₁₇ is a group (d33); and
X₁₈ and X₁₉ are independently as defined for X₁-X₈;

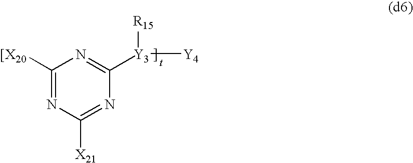

wherein
X₂₀ and X₂₁ are independently as defined for X₁-X₈;

### Y₃ is N, O or S;

If Y₃ is O or S, then R₁₅ is an electron pair;
If Y₃ is N, then R₁₅ is H, C₁-C₄alkyl or a group of formula (d33);
t is 3;
Y₄ is a trivalent inorganic or organic residue;

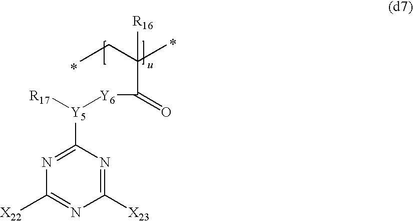

wherein

### R₁₆ is H or CH₃;

### Y₅ is N, O or S;

If Y₅ is O or S, then R₁₇ is an electron pair
If Y₅ is N, then R₁₇ is H or a group of formula (d33);
Y₆ is a direct bond, —NH— or —CH₂CH₂—N— or —CH₂CH₂—O—; and
X₂₂ and X₂₃ have the same meaning as defined for X₁-X₈.

Compounds of formula (d2) are preferred.

For instance, X₁-X₈ are as defined above.

In particular the active material comprises a compound of formulae (e1) to (e7)

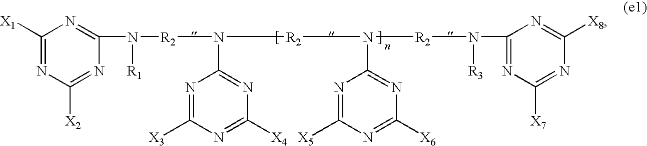

wherein
n is a number from 0-10
R₁ and R₃ are independently H or a group

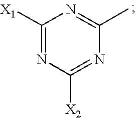

### R₂ is —CH₂—CH₂—;

X₁-X₈ independently of one another are groups of the formulas (e31)-(e34), preferably (e31) or (e32),

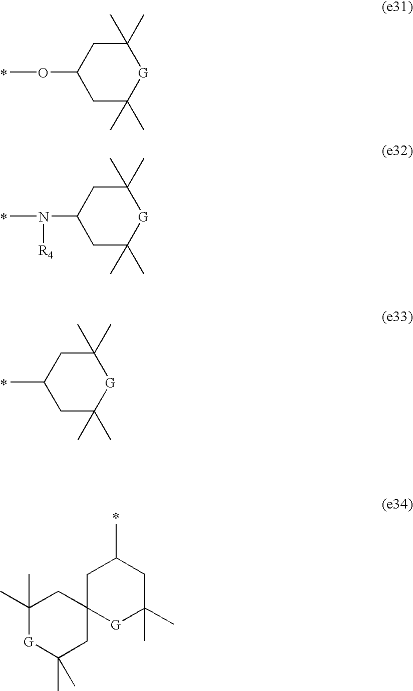

additionally one or more of X₁-X₈ are C₁-C₁₂alkylamino, C₁-C₁₂dialkylamino, halogen, NH₂, C₁-C₁₂alkenylamino or C₁-C₁₂alkynylamino;

### G is

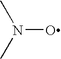

R₄ is hydrogen or a group of formula (e33);

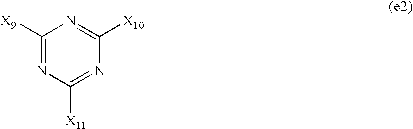

X₉-X₁₁ independently of one another are as defined for X₁-X₈;

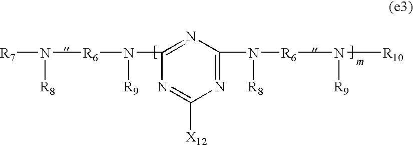

wherein
m is 0-10
R₆ is —CH₂CH₂— or a direct bond;
R₇, R₈, R₉ and R₁₀ are a group

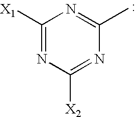

and X₁₂ has the meaning as defined for X₁-X₈;

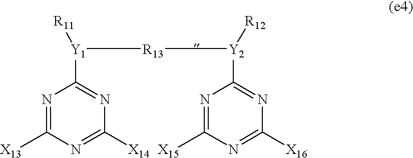

wherein

### Y₁ and Y₂ are N, O or S

R₁₃ is —CH₂CH₂— or a direct bond if Y₁ and Y₂ are N or S;
If Y₁ or Y₂ are O or S, then R₁₁ or R₁₂ are an electron pair;
If Y₁ and Y₂ are N, then R₁₁ and R₁₂ are a group of formula (e33) or a group

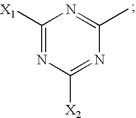

X₁₃-X₁₆ are independently as defined for X₁-X₈;

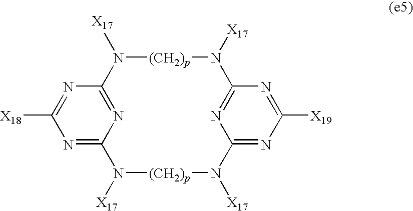

wherein
p is a number 2-6
X₁₇ is a group (e33); and
X₁₈ and X₁₉ are independently as defined for X₁-X₈;

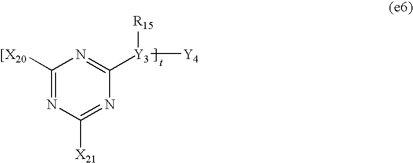

wherein
X₂₀ and X₂₁ are independently as defined for X₁-X₈,

### Y₃ is N, O or S;

If Y₃ is O or S, then R₁₅ is an electron pair;
If Y₃ is N, then R₁₅ is H or a group of formula (e33);
t is 3;
Y₄ is a trivalent inorganic or organic residue;

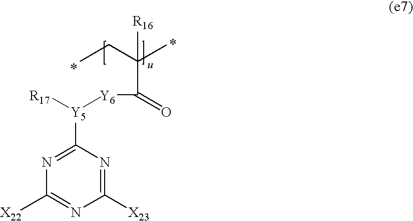

wherein

### R₁₆ is H;

### Y₅ is N or O;

If Y₅ is O, then R₁₇ is an electron pair
If Y₅ is N, then R₁₇ is H or a group of formula (e33);
Y₆ is a direct bond, —NH—, —CH₂CH₂—N— or —CH₂CH₂—O—; and
X₂₂ and X₂₃ have the same meaning as defined for X₁-X₈.

For instance, X₁-X₈ are as defined above.

For example, X₁-X₈ are independently of one another groups of formula (e31) or (e32); additionally one or more of X₁-X₈ are C₁-C₁₂alkylamino, C₁-C₁₂dialkylamino, halogen, NH₂, C₁-C₁₂alkenylamino or C₁-C₁₂alkynylamino.

For instance, X₁-X₈ are independently of one another groups of formula (e31) or (e32); additionally one or more of X₁-X₈ are C₁-C₁₂alkylamino, halogen, NH₂ or C₁-C₁₂alkynylamino.

For instance, the active material is of formula (e2).

The active material is preferably a compound of formula (e2) wherein

X₉, X₁₀, and X₁₁ are independently of one another groups of formula (e31) or (e32);
additionally one or more of X₉-X₁₁ are C₁-C₁₂alkylamino, C₁-C₁₂dialkylamino, halogen, NH₂, C₁-C₁₂alkenylamino or C₁-C₁₂alkynylamino; and
R₄ is H or a group of formula (e33).

More preferably the active material is a compound of formula (e2) wherein

X₉, X₁₀, and X₁₁ are independently of one another groups of formula (e31) or (e32);
additionally one or more of X₉-X₁₁ are C₁-C₁₂alkylamino, halogen, NH₂ or C₁-C₁₂alkynylamino; and
R₄ is H or a group of formula (e33).

The precursor compounds of the nitroxides (sterically hindered NH compounds) are essentially known and partially commercially available. All of them can be prepared by known processes. Their preparation is disclosed, for example, in:

U.S. Pat. No. 5,679,733, U.S. Pat. No. 3,640,928, U.S. Pat. No. 4,198,334, U.S. Pat. No. 5,204,473, U.S. Pat. No. 4,619,958, U.S. Pat. No. 4,110,306, U.S. Pat. No. 4,110,334, U.S. Pat. No. 4,689,416, U.S. Pat. No. 4,408,051, SU-A-768,175 (Derwent 88-138,751/20), U.S. Pat. No. 5,049,604, U.S. Pat. No. 4,769,457, U.S. Pat. No. 4,356,307, U.S. Pat. No. 4,619,956, U.S. Pat. No. 5,182,390, GB-A-2,269,819, U.S. Pat. No. 4,292,240, U.S. Pat. No. 5,026,849, U.S. Pat. No. 5,071,981, U.S. Pat. No. 4,547,538, U.S. Pat. No. 4,976,889, U.S. Pat. No. 4,086,204, U.S. Pat. No. 6,046,304, U.S. Pat. No. 4,331,586, U.S. Pat. No. 4,108,829, U.S. Pat. No. 5,051,458, WO-A-94/12,544 (Derwent 94-177,274/22), DD-A-262,439 (Derwent 89-122,983/17), U.S. Pat. No. 4,857,595, U.S. Pat. No. 4,529,760, U.S. Pat. No. 4,477,615, CAS 136, 504-96-6, U.S. Pat. No. 4,233,412, U.S. Pat. No. 4,340,534, WO-A-98/51,690 and EP-A-1,803, in particular U.S. Pat. No. 4,442,250 or U.S. Pat. No. 6,046,304.

The oxidation may be carried out in analogy to the oxidation of 4-hydroxy-2,2,6,6-tetramethylpiperidine described in U.S. Pat. No. 5,654,434 with hydrogen peroxide. Another also suitable oxidation process is described in WO 00/40550 using peracetic acid.

An exhaustive description of the nitroxide chemistry can be found, for example, in L. B. Volodarsky, V. A. Reznikov, V. I. Ovcharenko.: “Synthetic Chemistry of Stable Nitroxides”, CRC Press, 1994.

The methods described in WO 2004/031150 can be used for the preparation of oxoammonium salts.

Not limiting examples of individual compounds, which are suitable as active materials in positive or negative electrodes are given below in Table A (TMP is 2,2,6,6-tetramethylpiperidine).

TABLE A
2 × 4-Amino-TMP

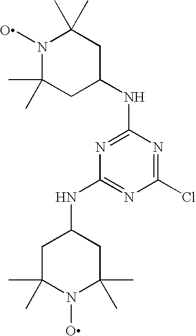

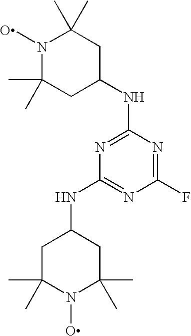

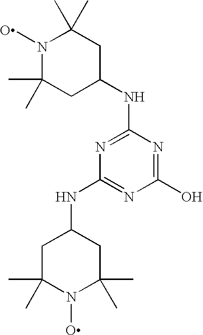

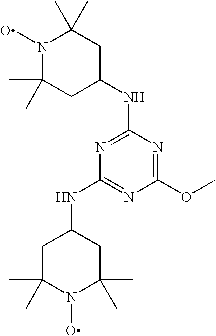

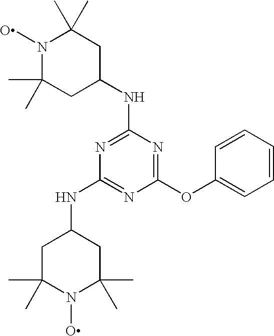

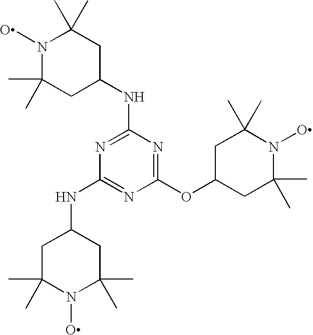

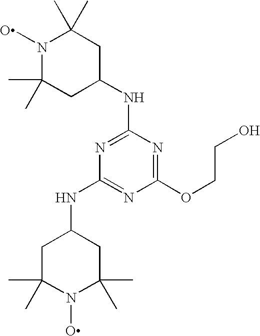

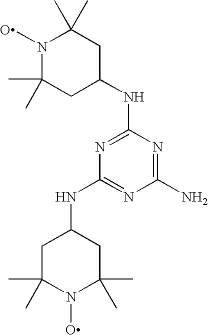

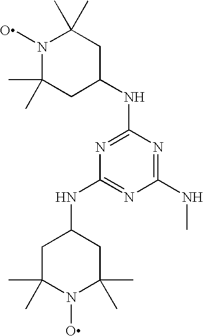

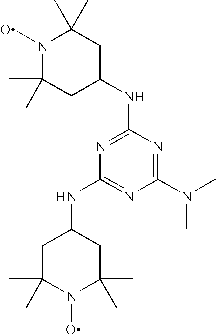

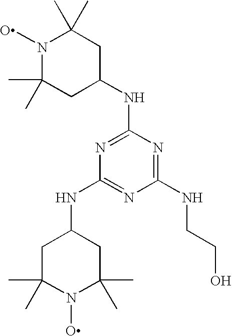

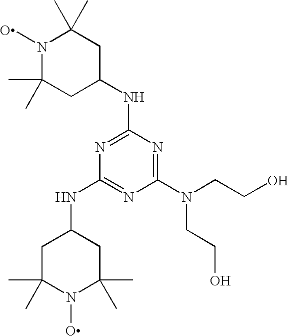

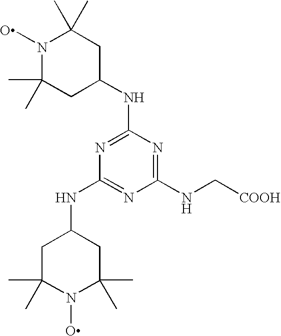

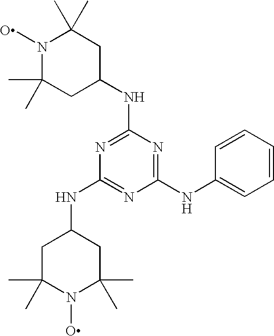

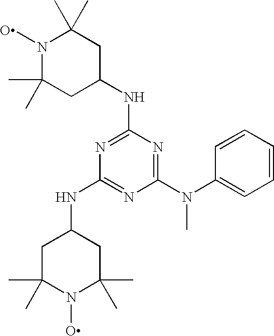

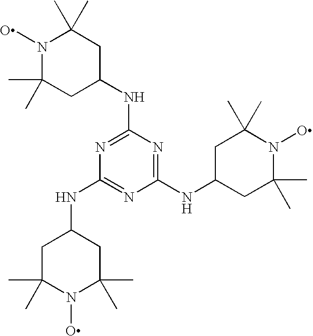

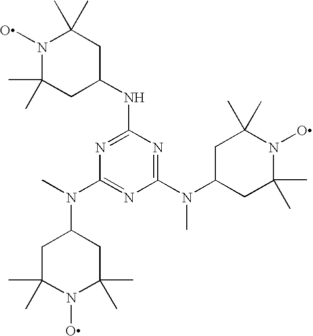

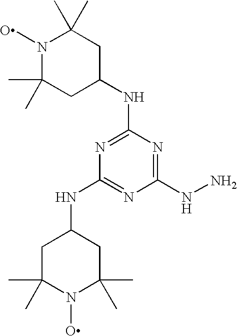

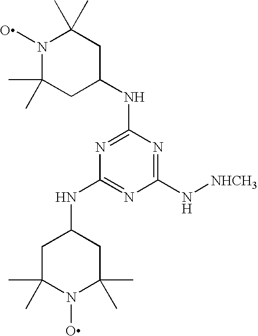

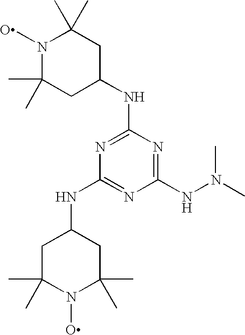

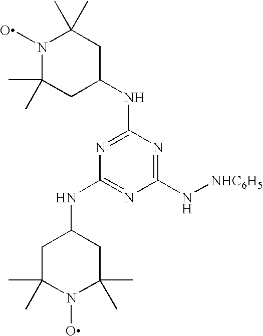

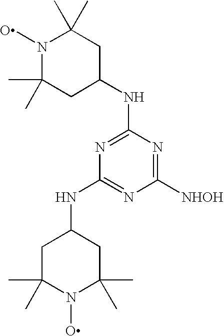

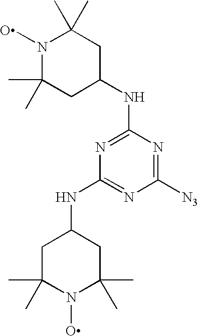

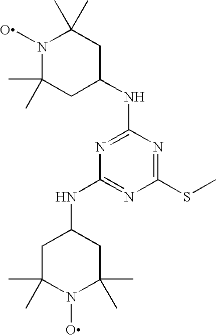

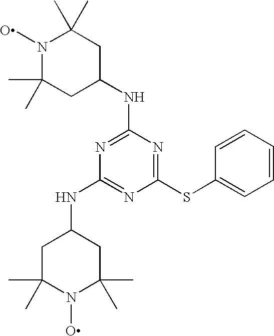

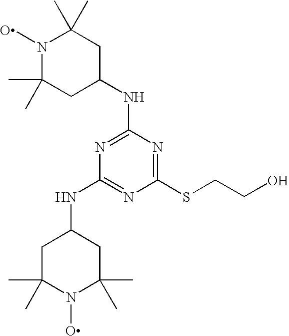

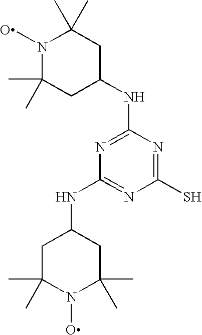

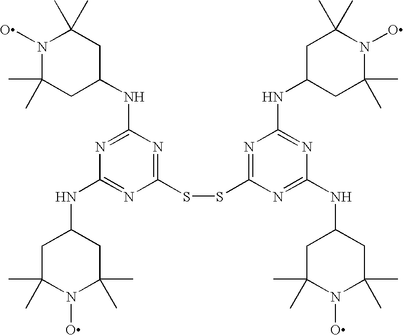

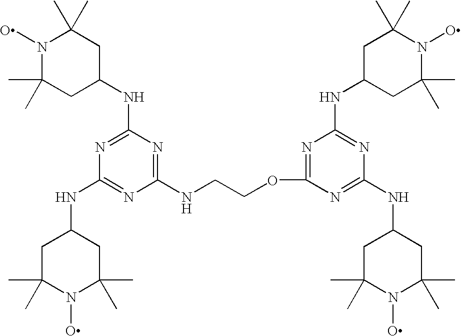

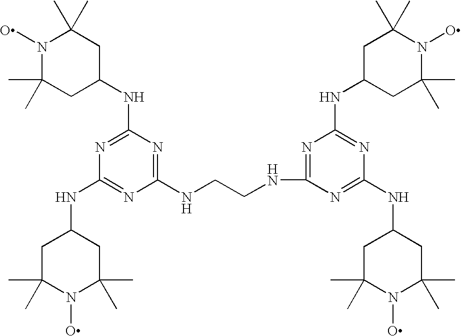

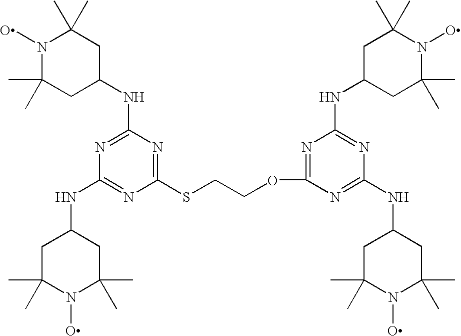

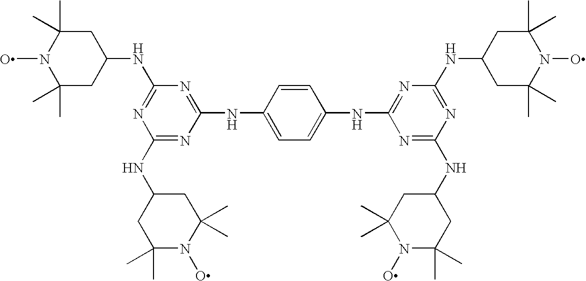

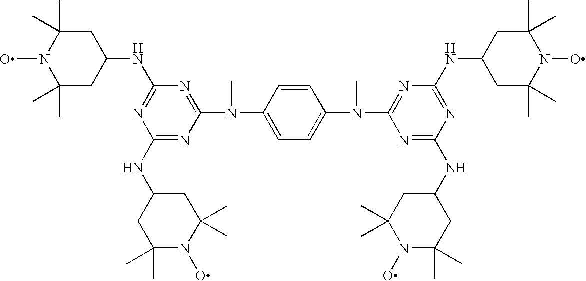

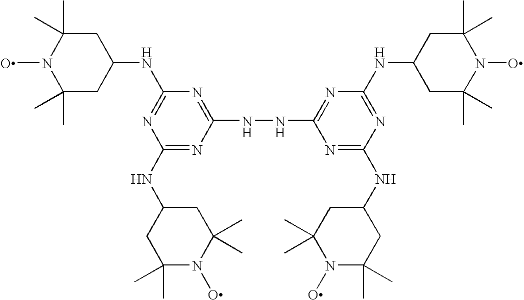

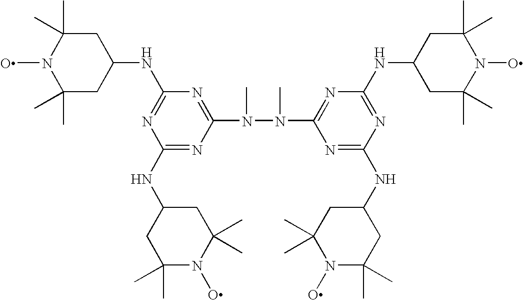

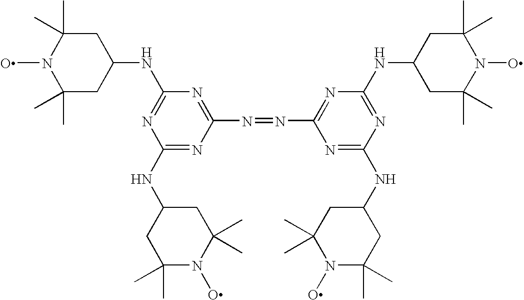

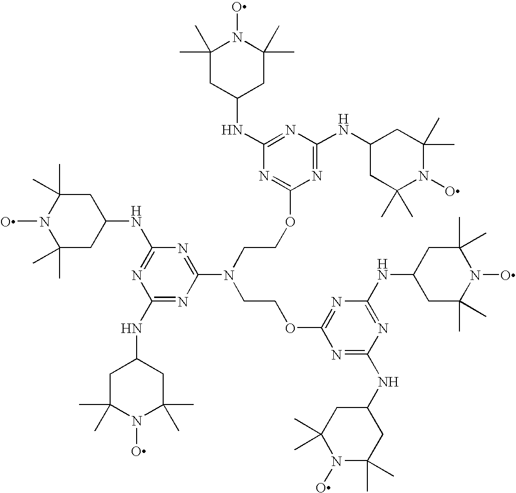

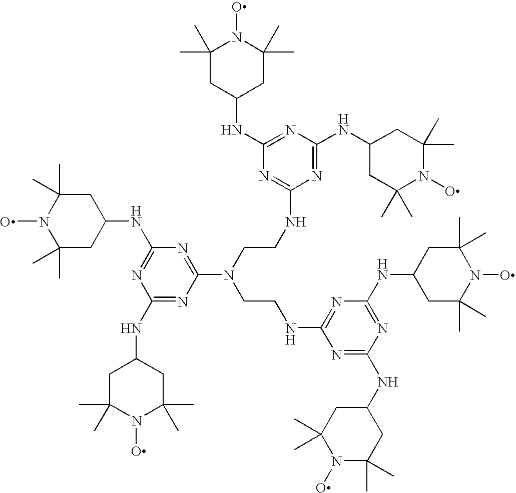

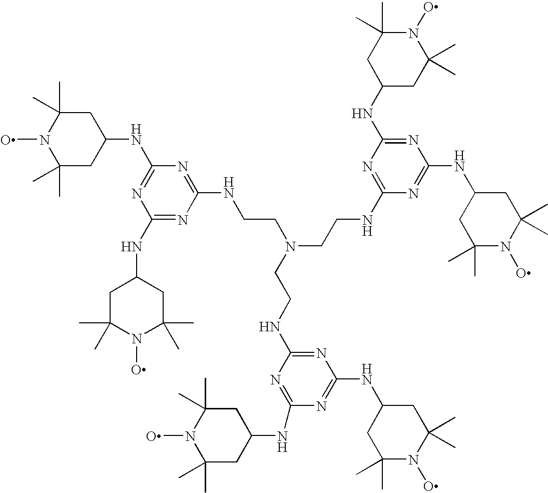

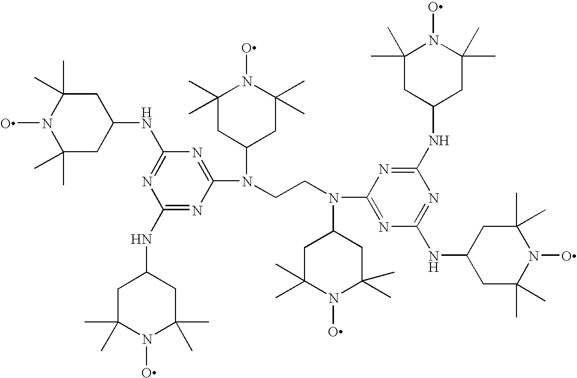

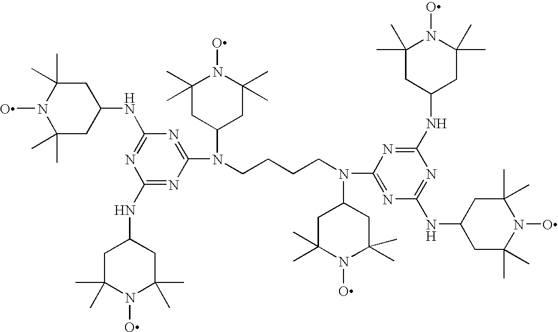

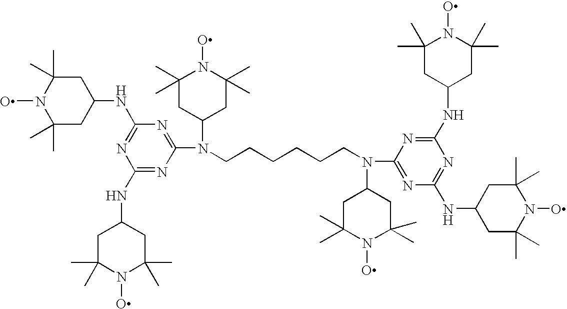

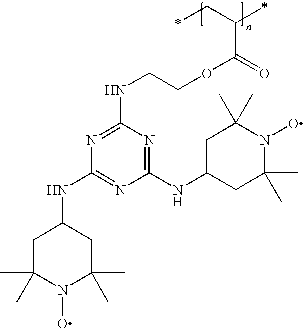

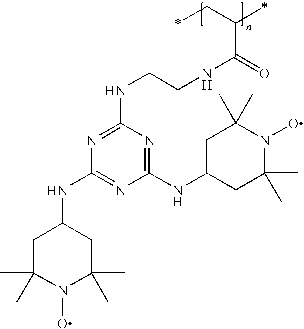

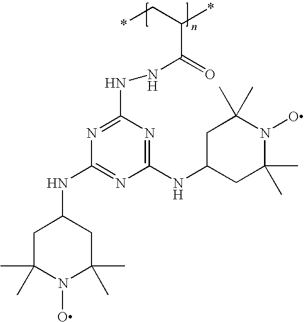

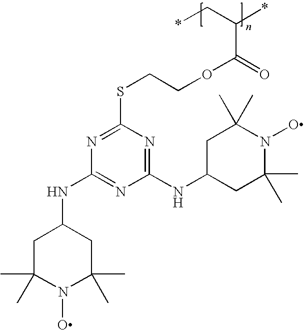

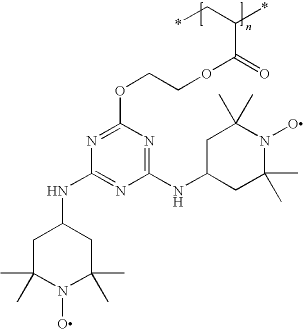

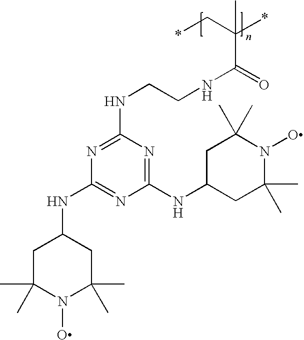

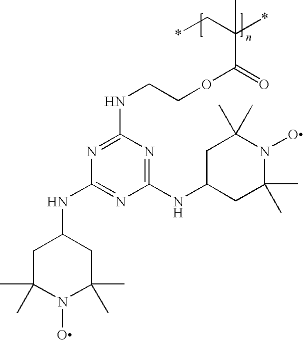

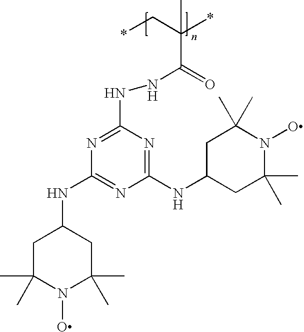

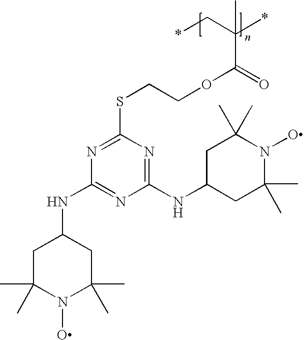

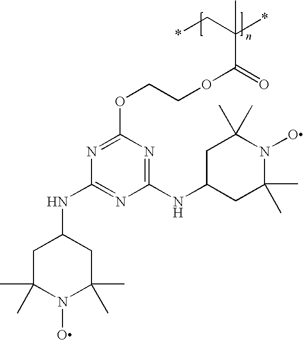

Amino + Hydroxy-TMP

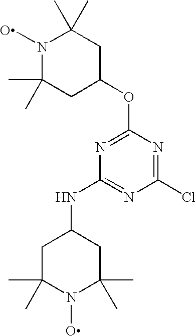

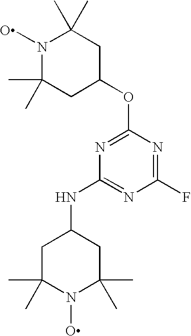

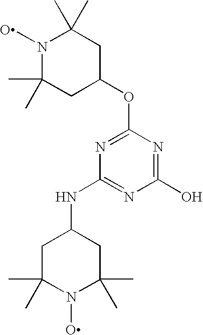

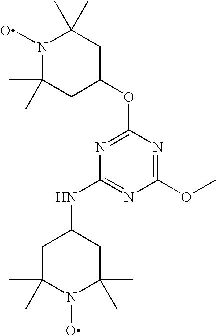

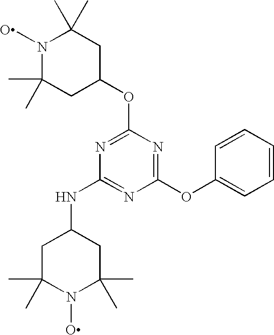

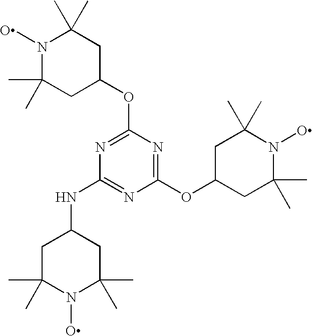

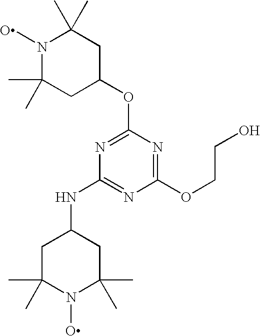

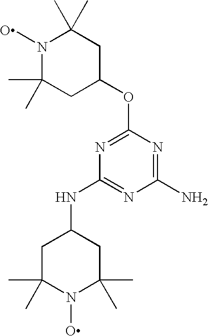

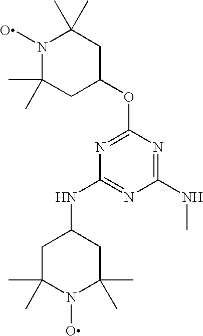

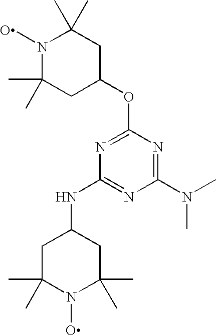

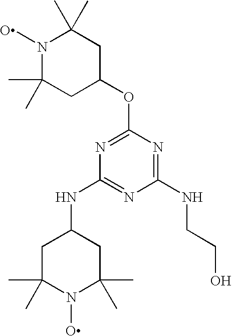

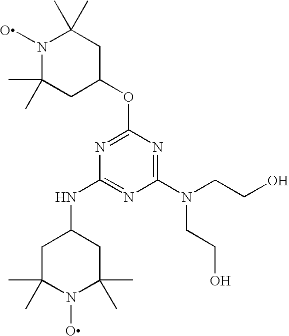

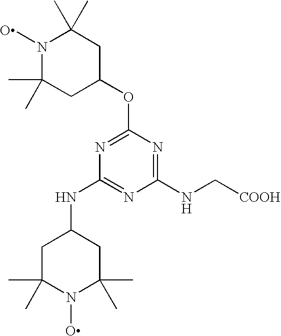

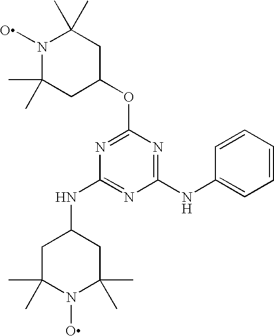

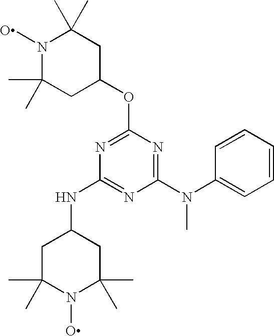

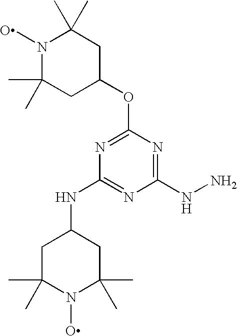

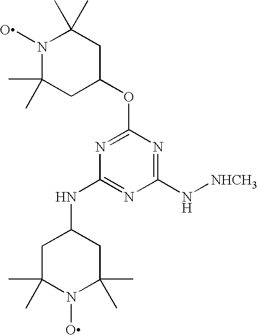

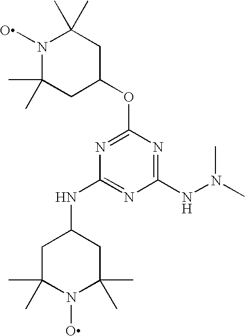

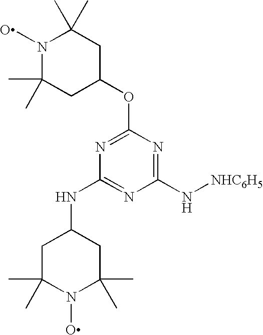

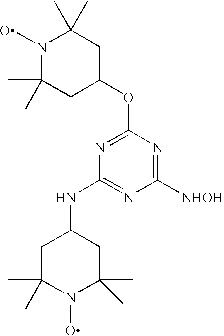

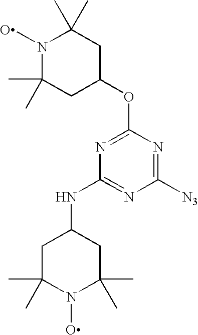

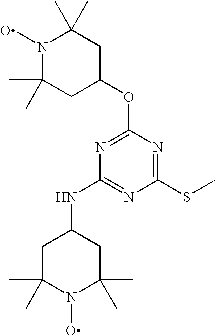

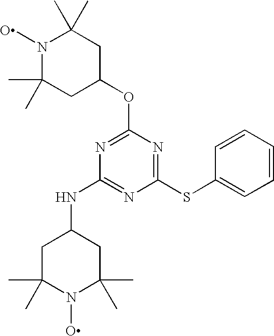

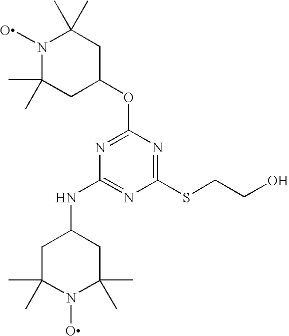

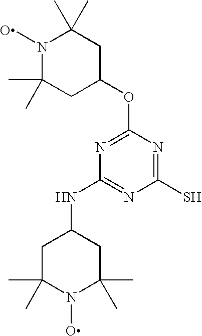

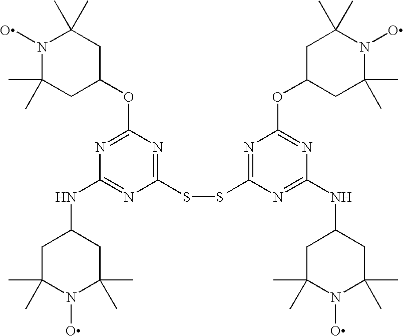

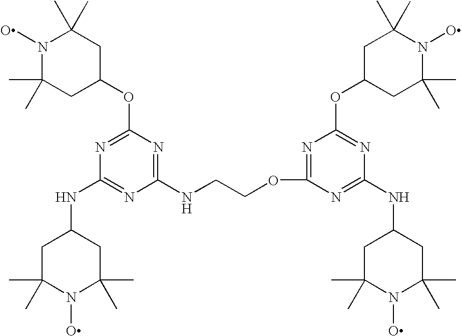

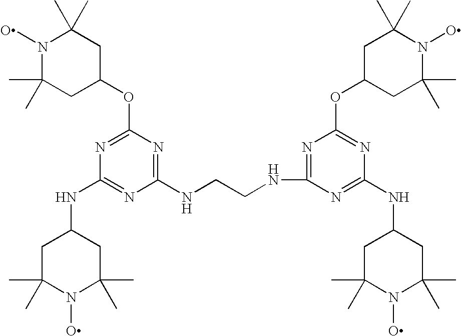

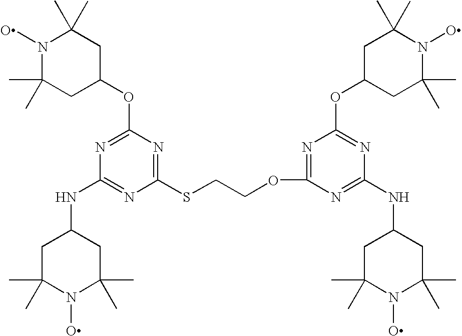

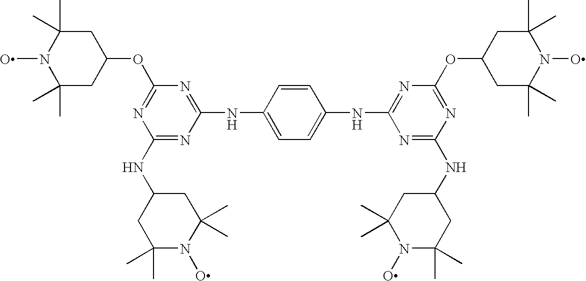

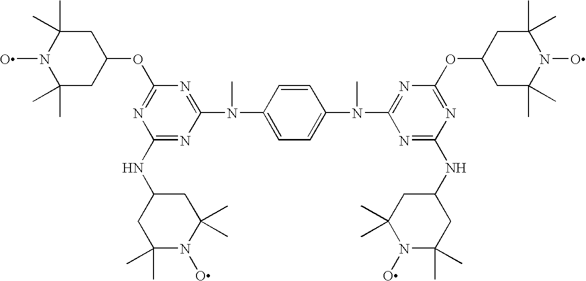

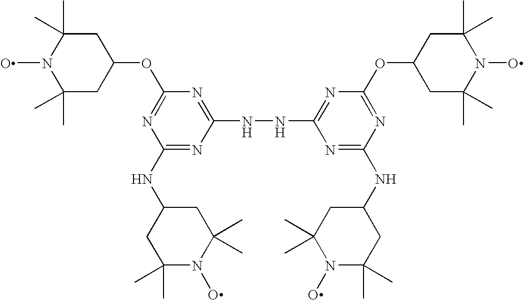

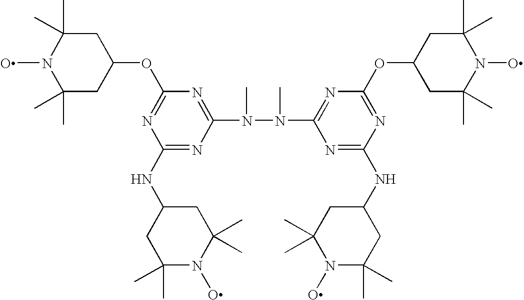

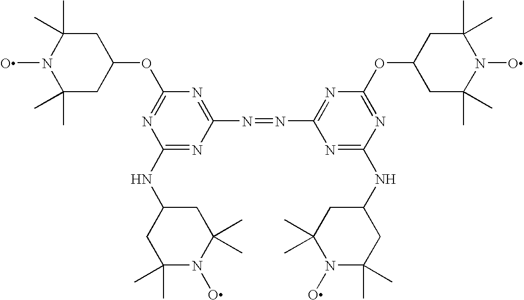

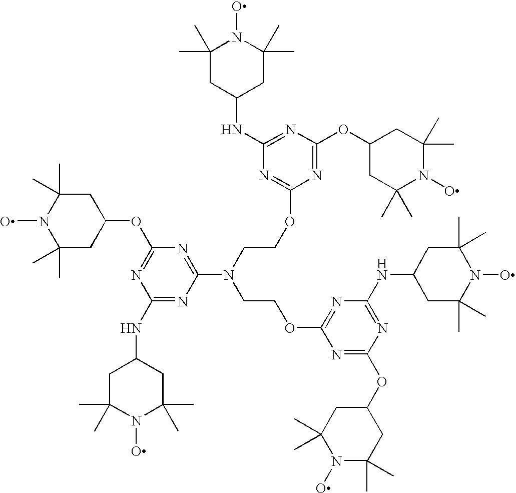

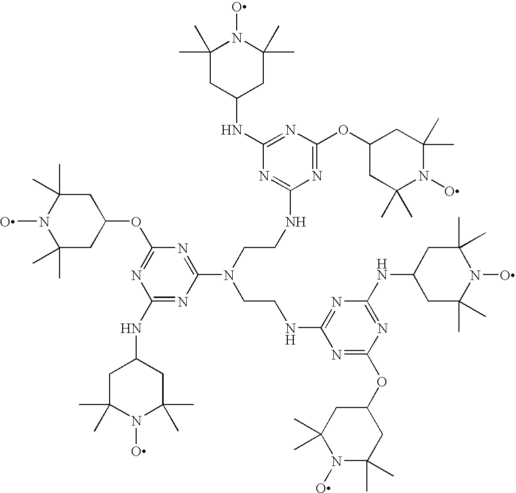

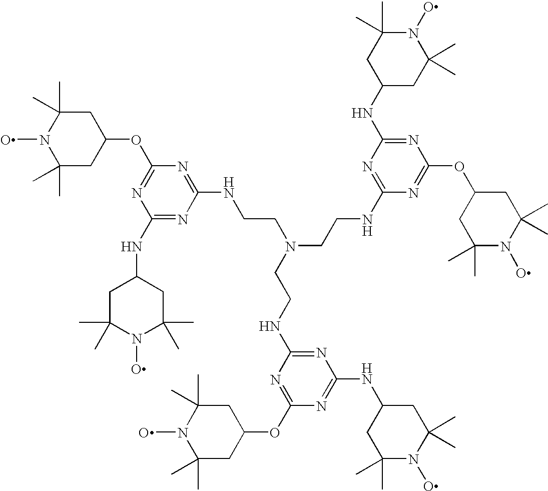

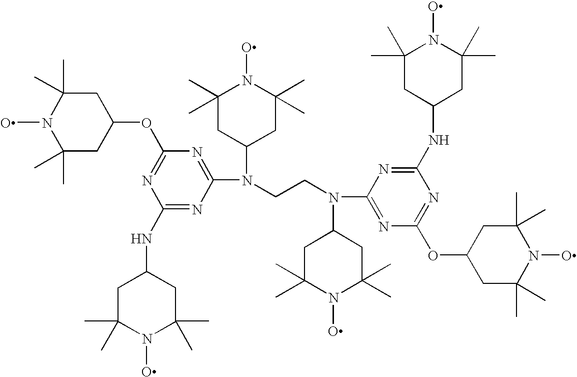

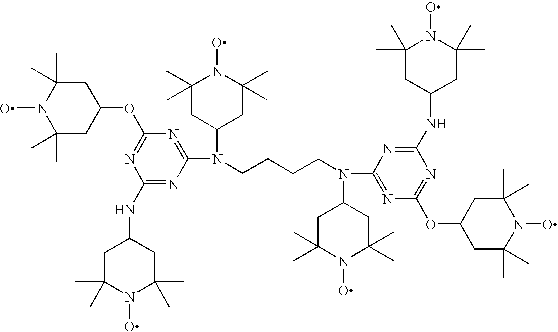

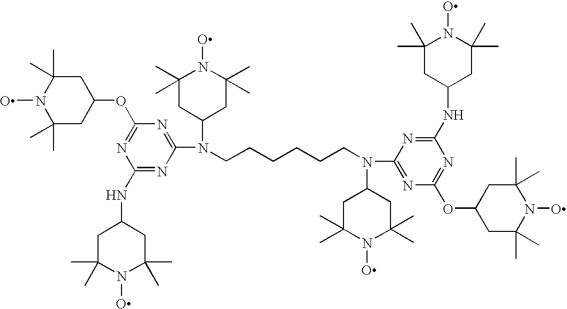

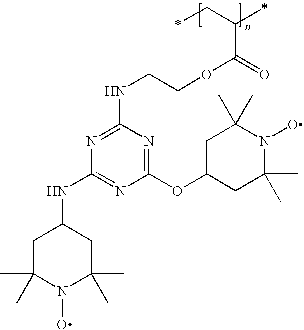

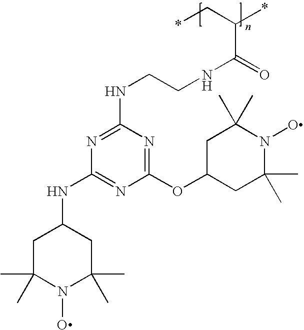

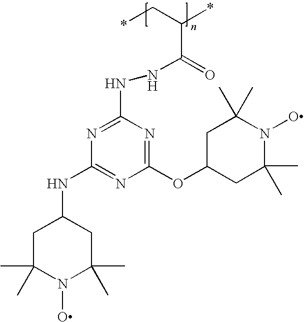

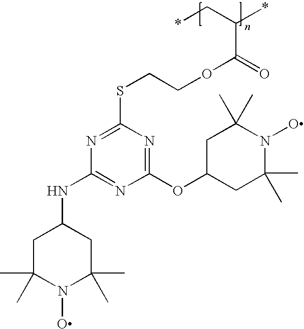

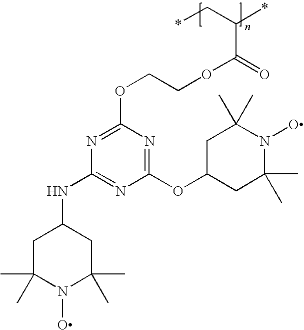

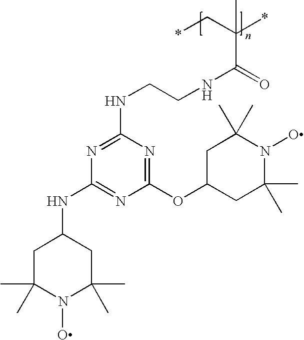

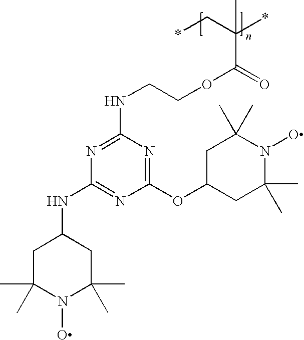

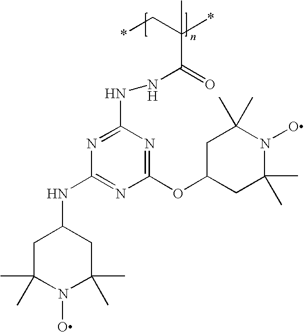

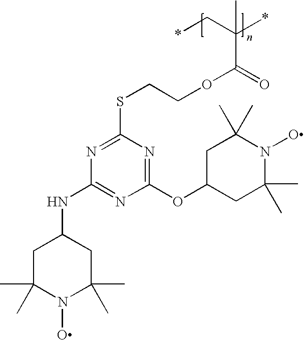

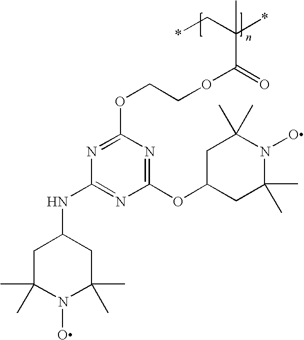

2 × 4-Hydroxy-TMP

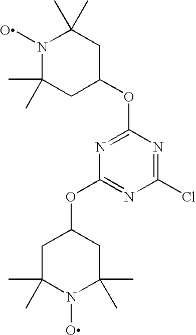

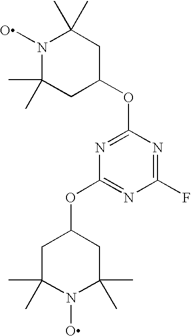

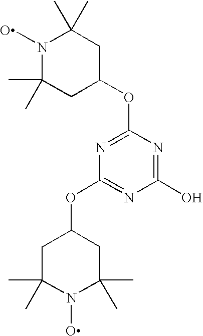

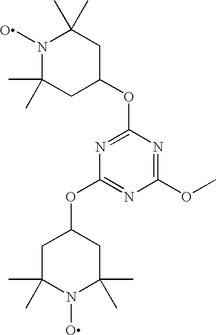

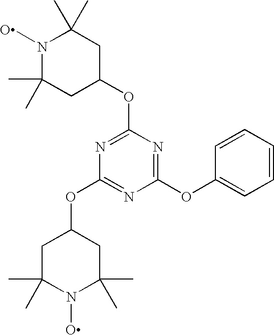

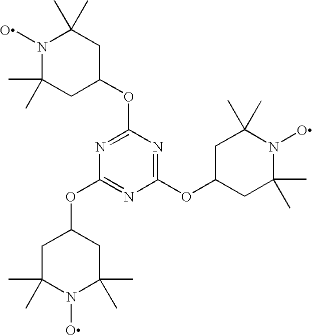

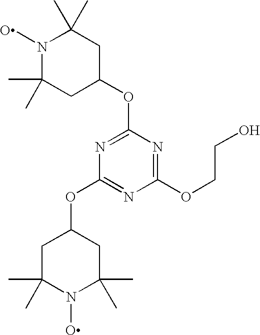

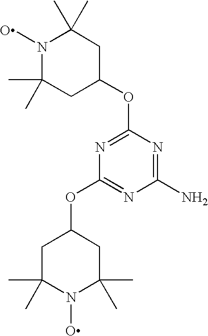

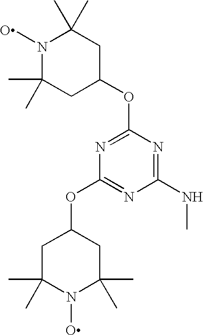

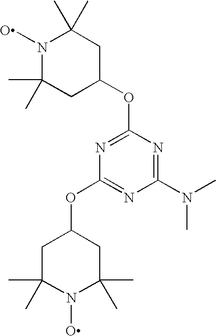

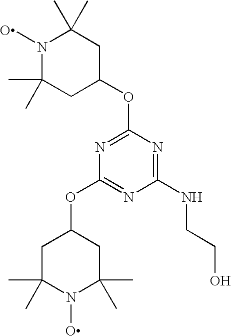

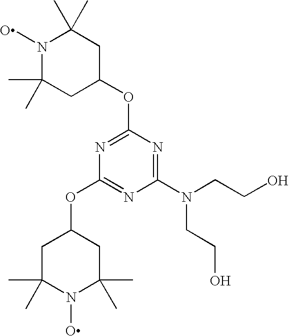

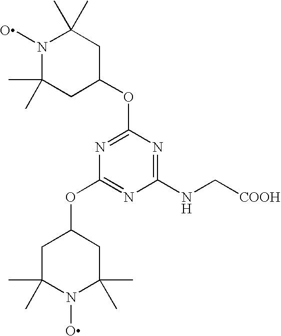

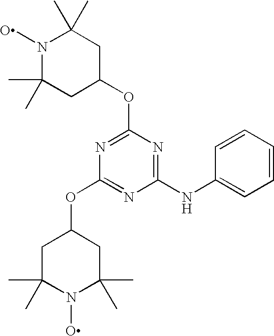

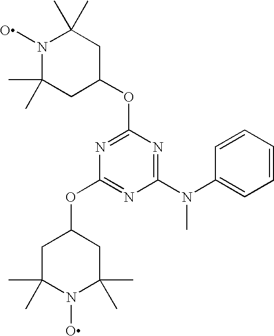

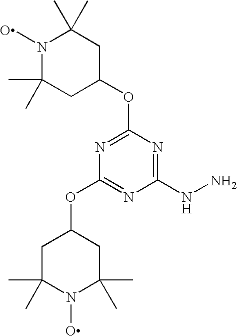

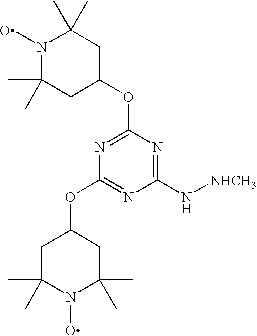

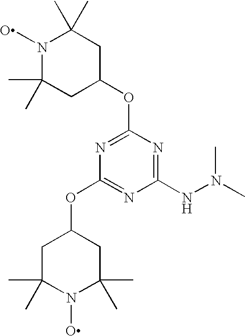

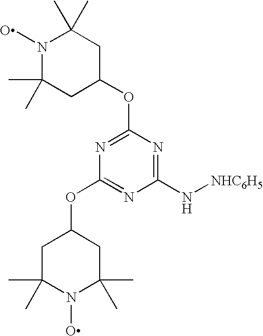

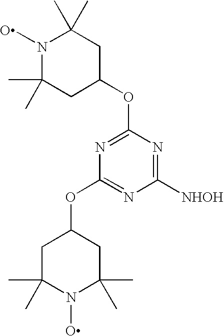

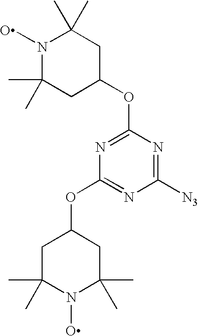

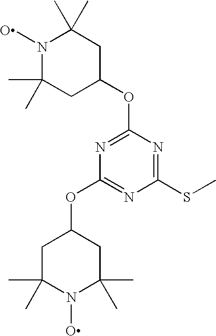

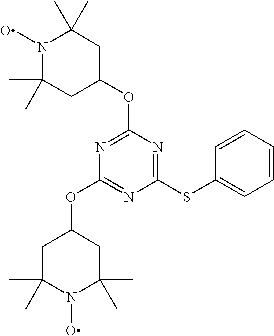

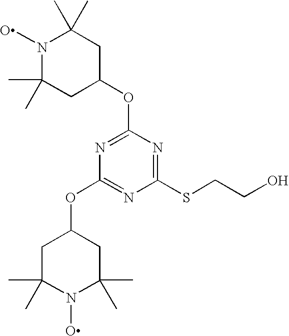

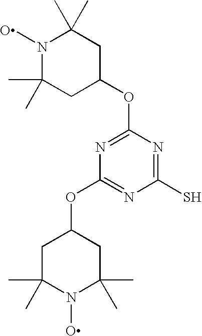

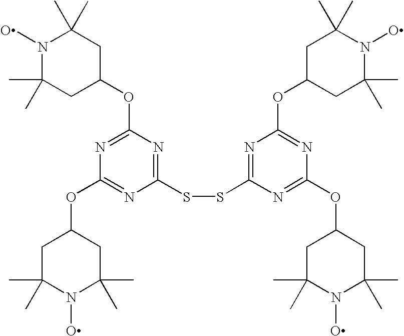

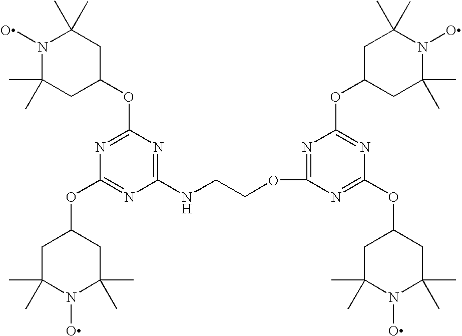

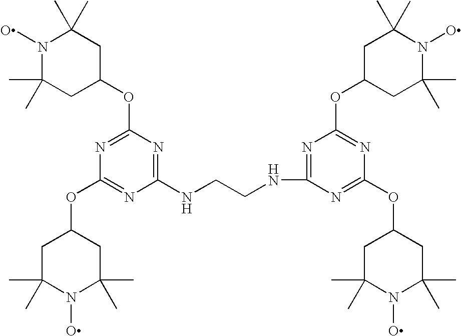

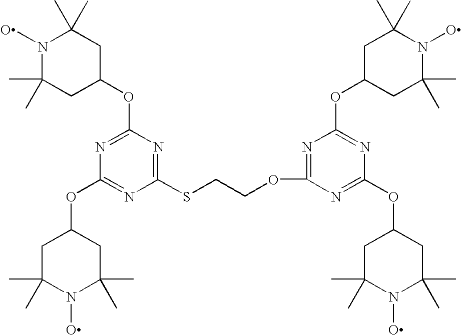

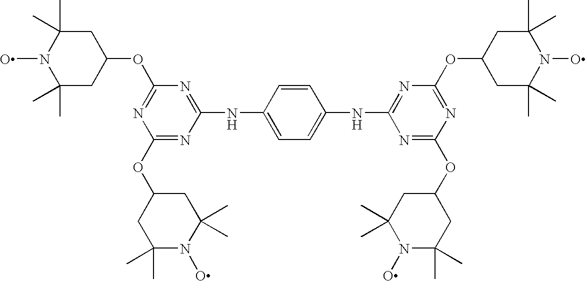

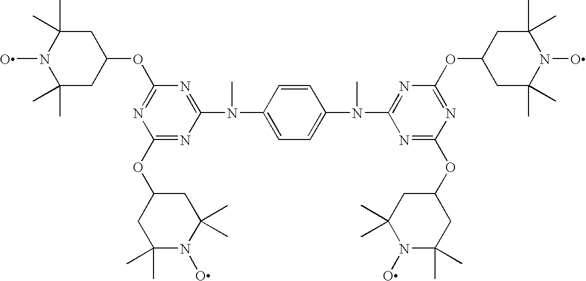

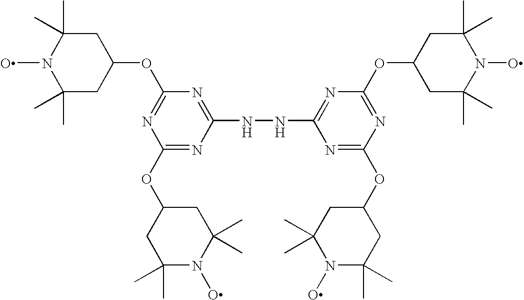

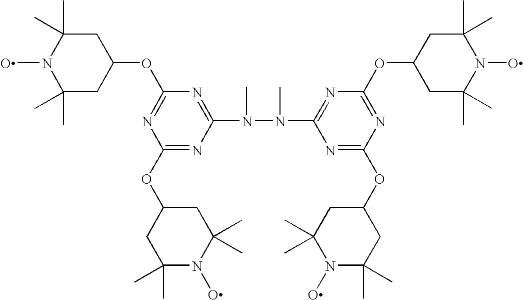

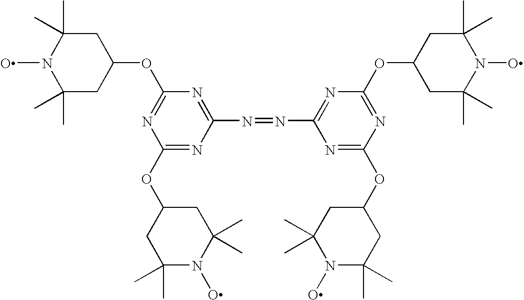

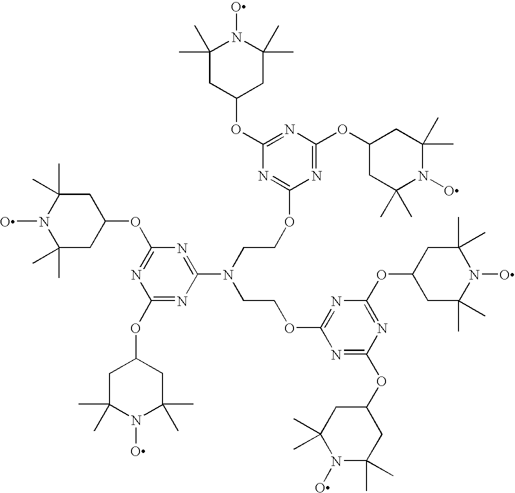

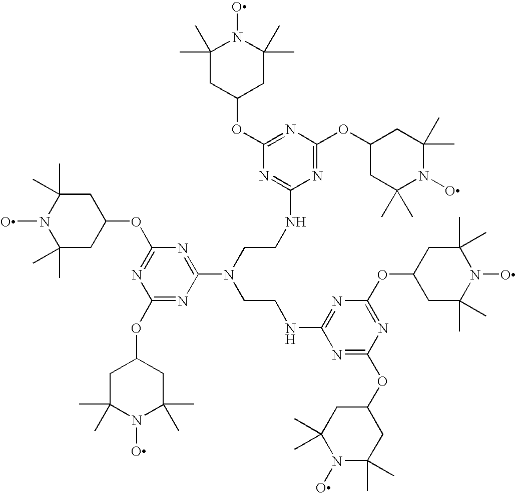

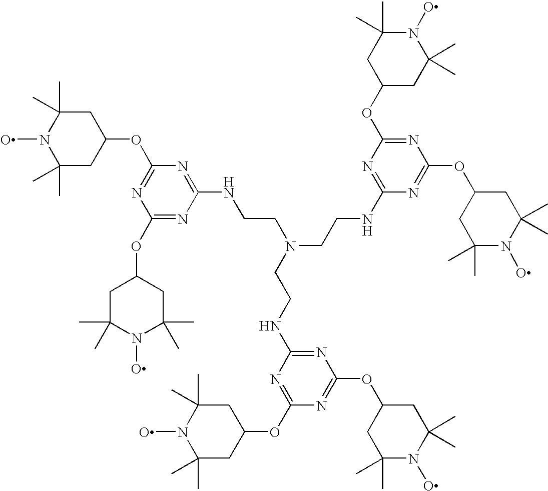

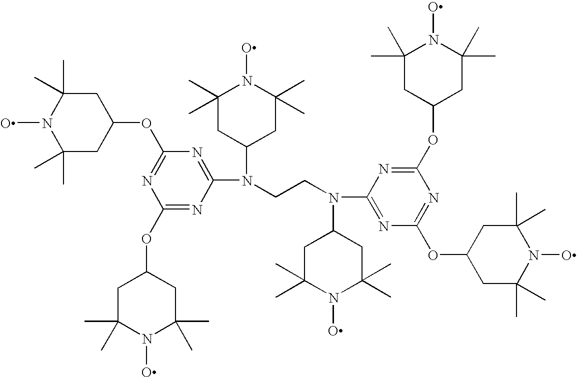

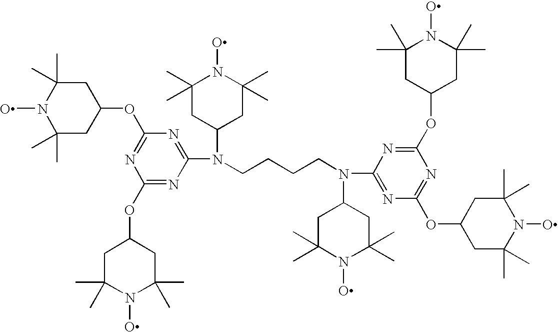

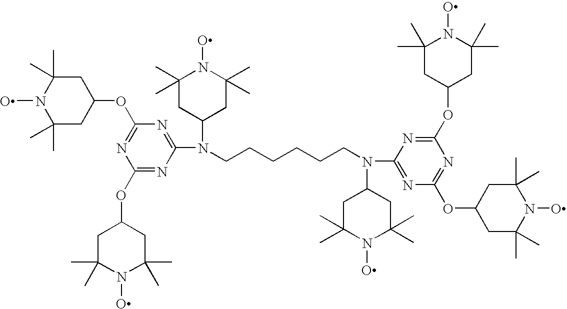

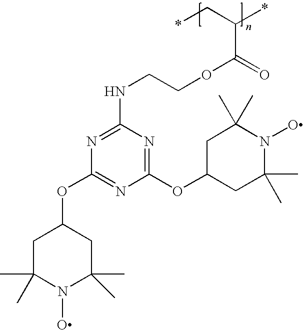

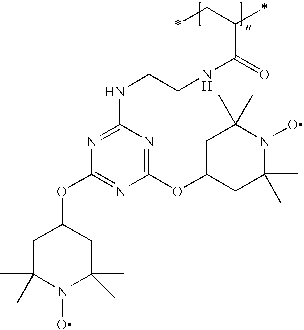

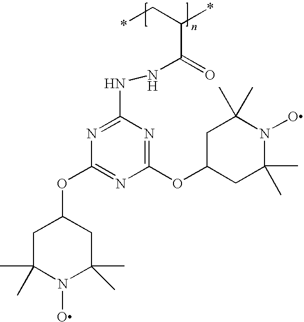

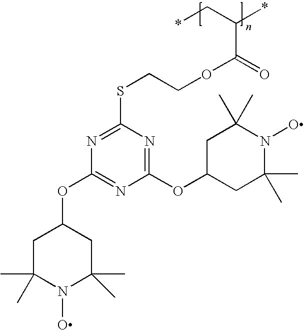

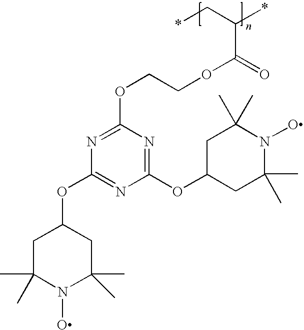

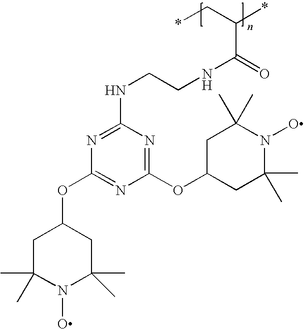

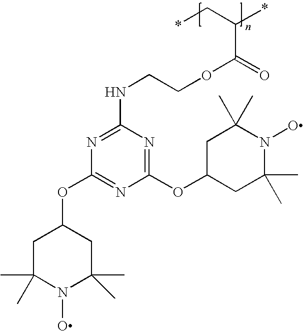

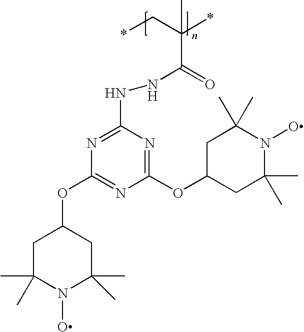

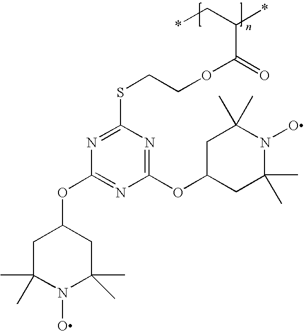

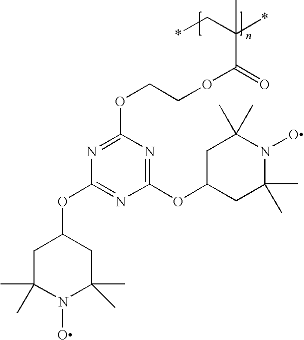

1 × Bis-TMP

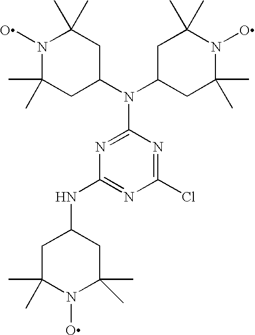

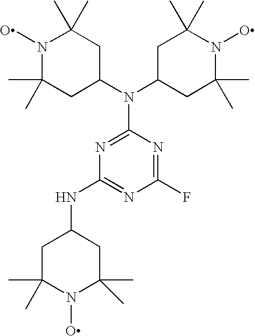

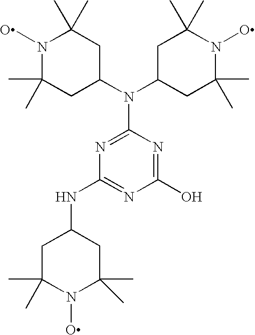

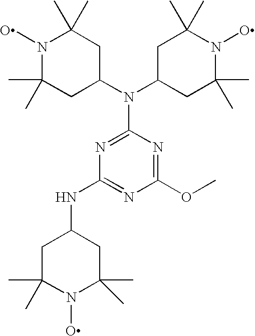

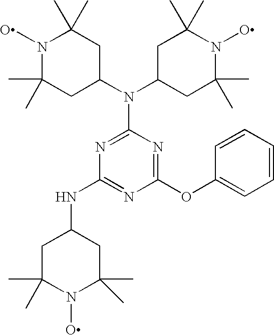

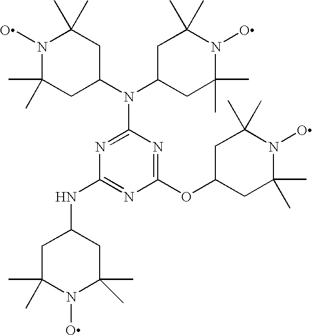

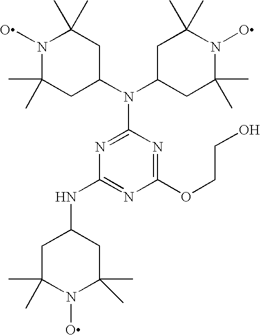

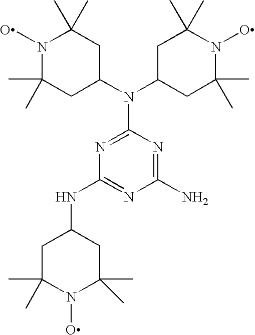

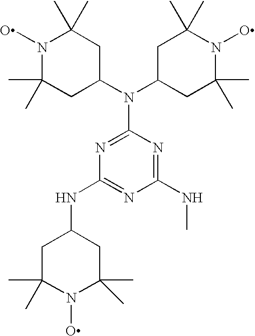

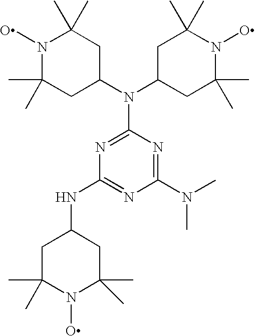

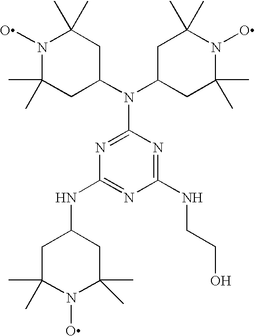

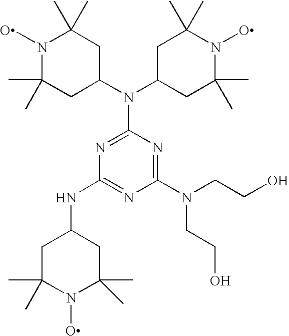

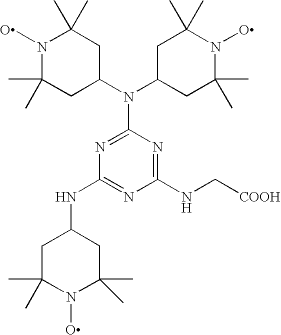

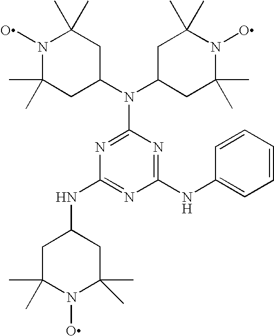

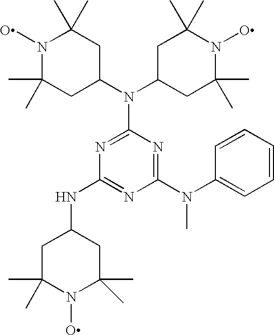

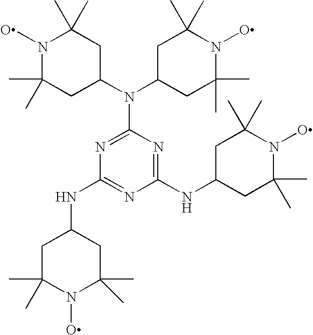

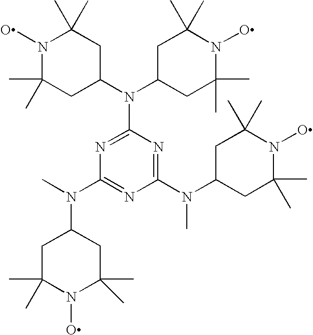

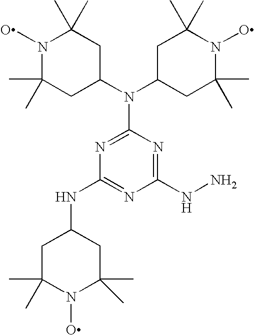

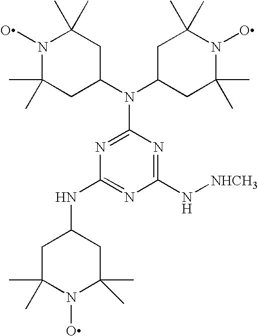

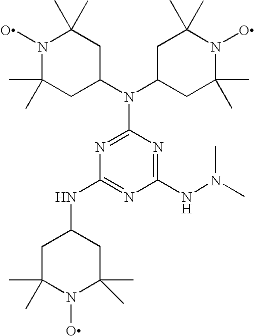

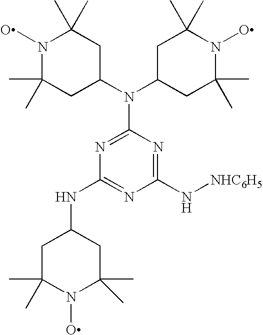

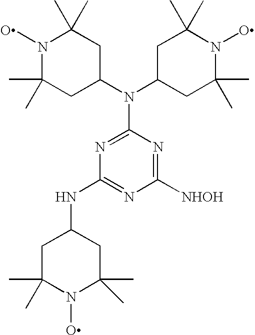

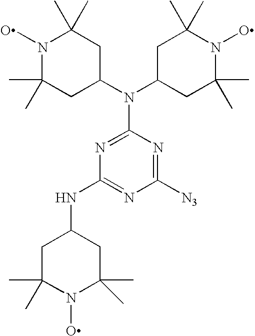

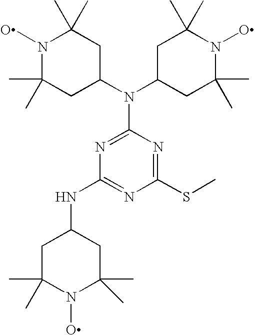

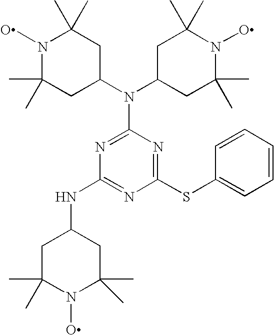

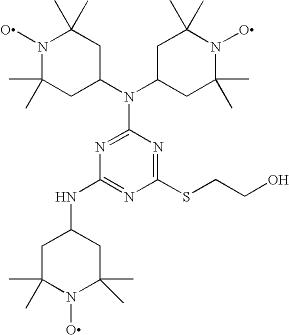

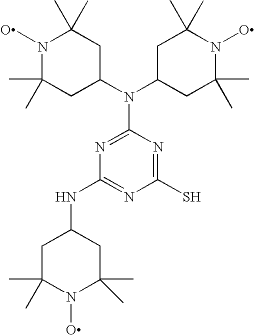

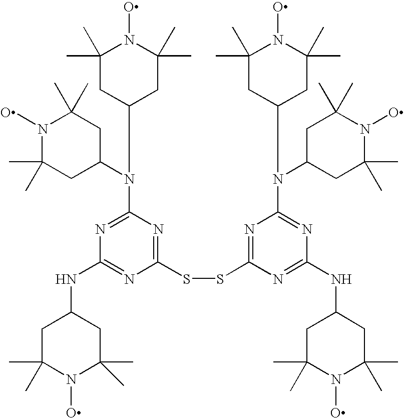

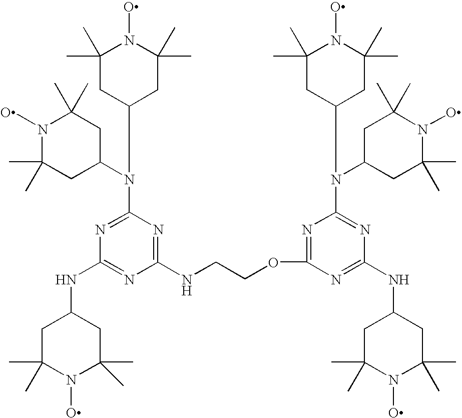

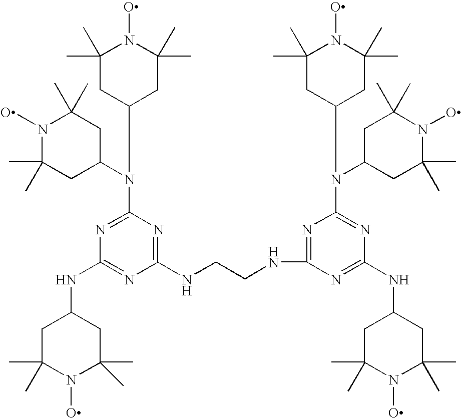

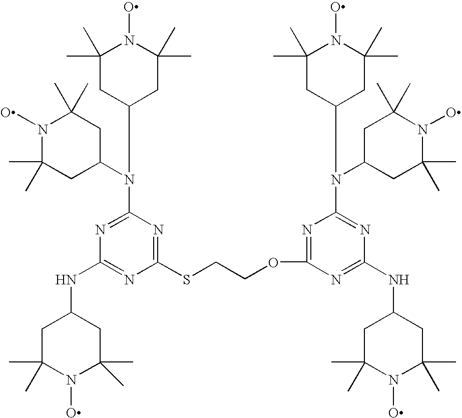

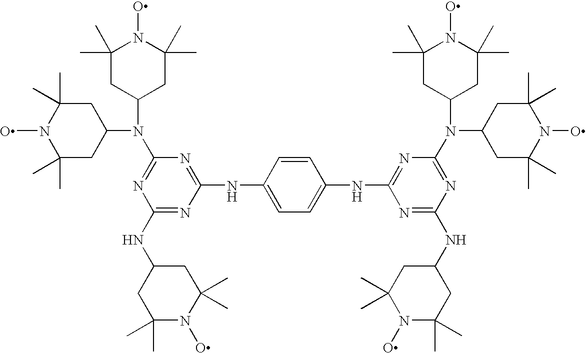

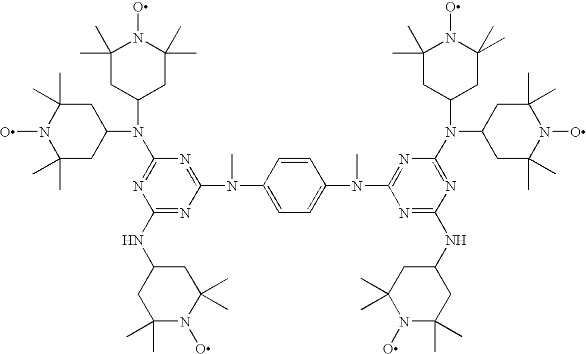

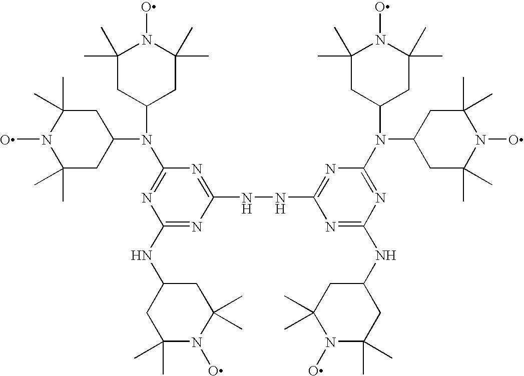

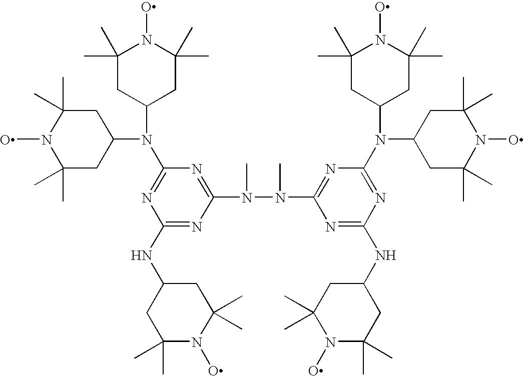

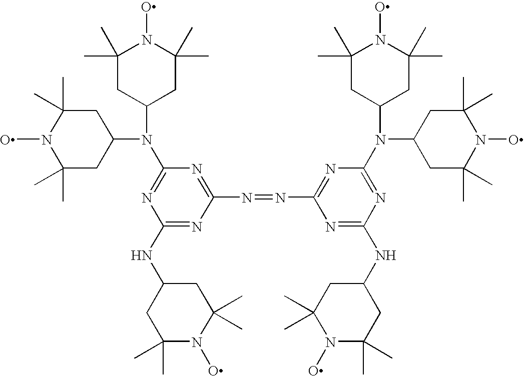

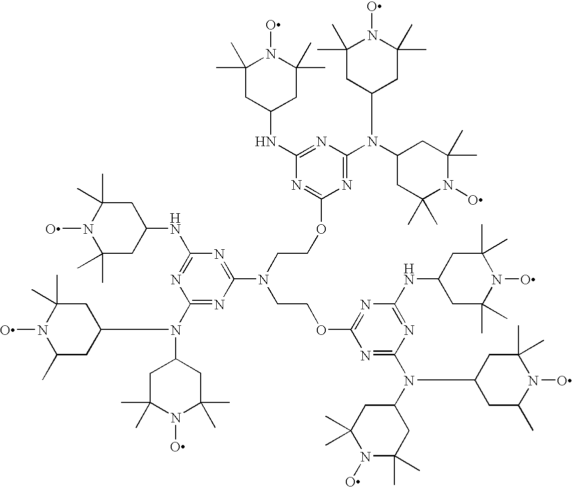

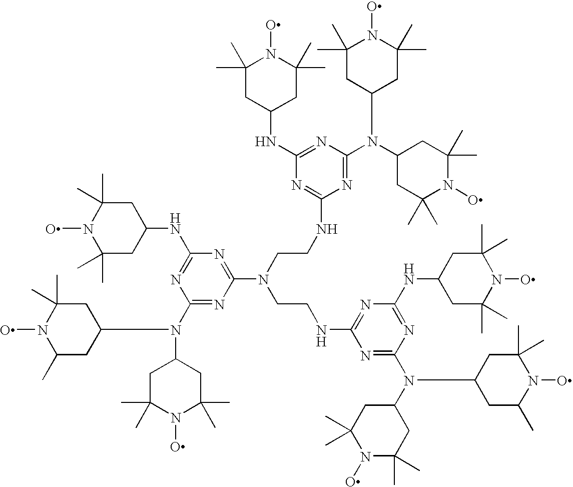

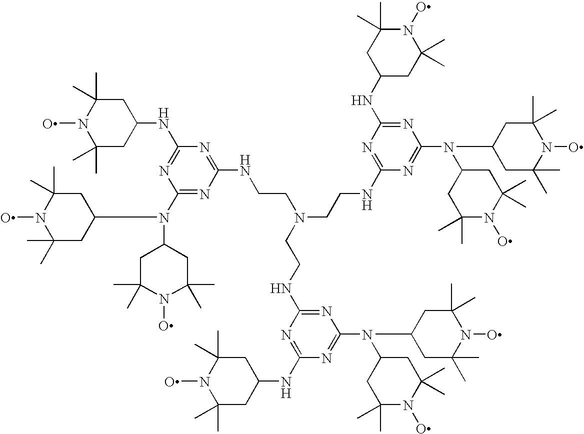

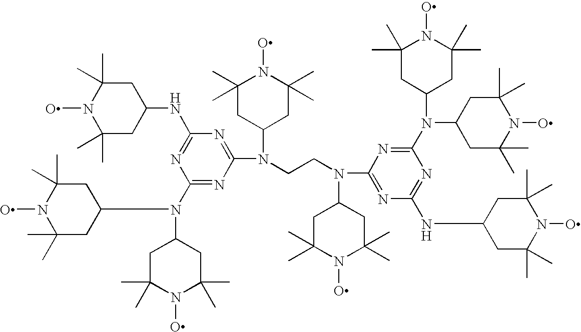

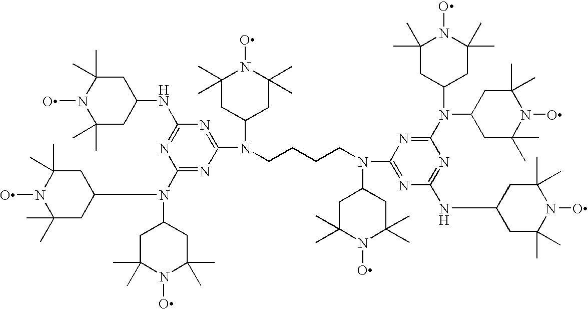

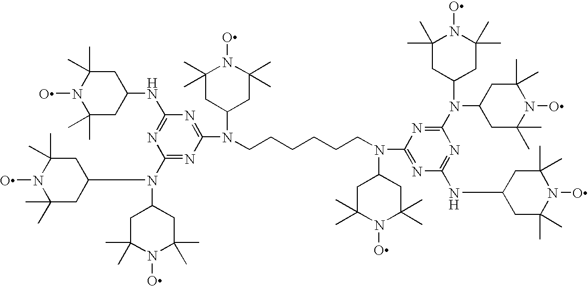

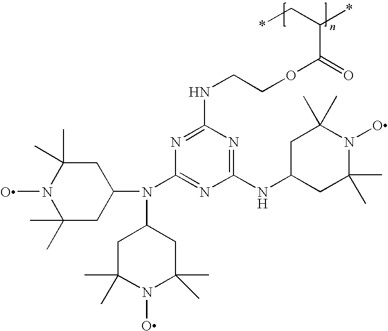

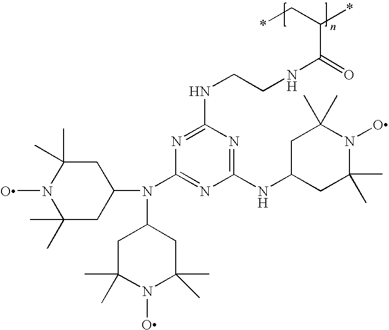

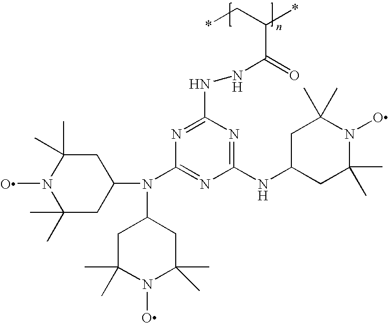

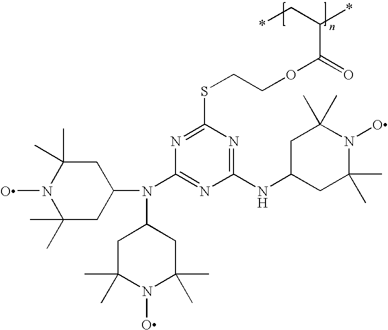

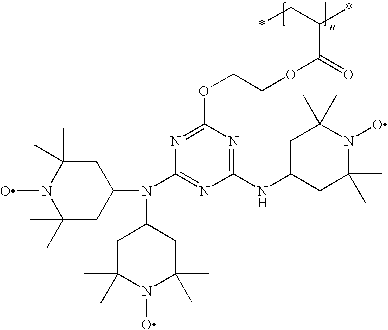

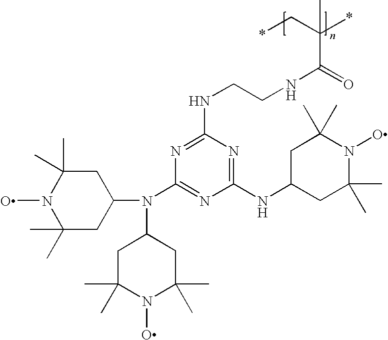

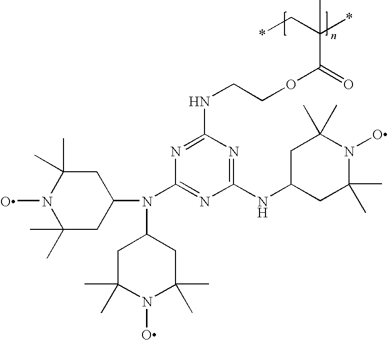

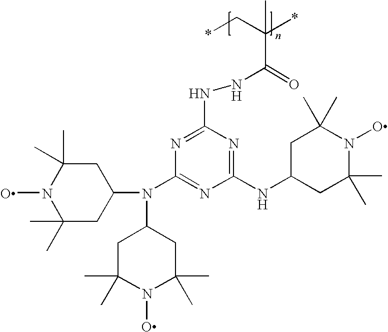

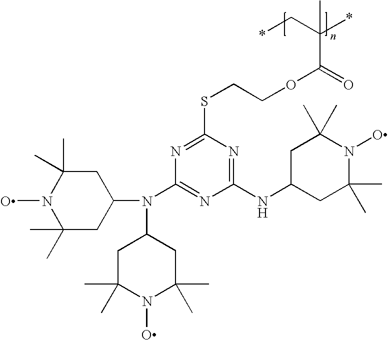

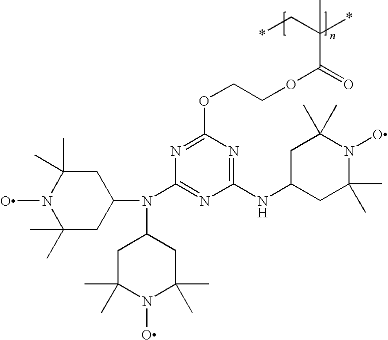

2 × Bis-TMP

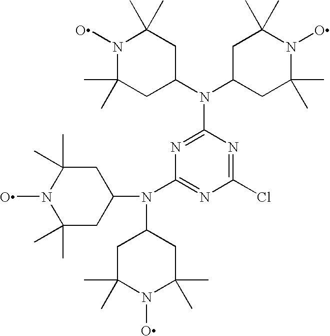

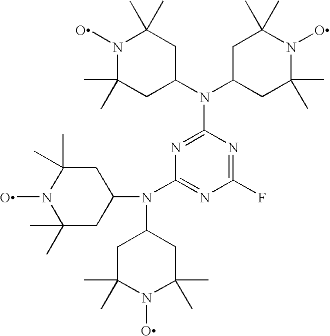

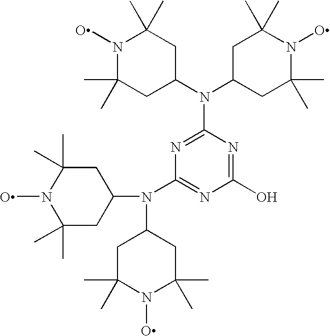

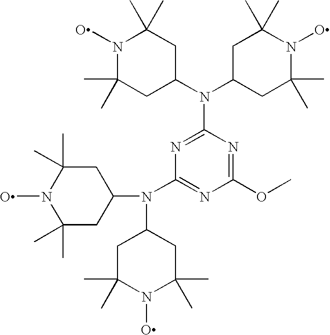

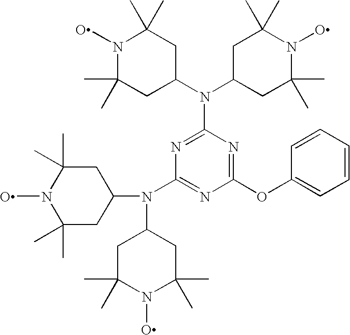

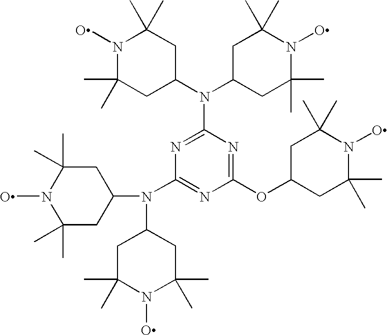

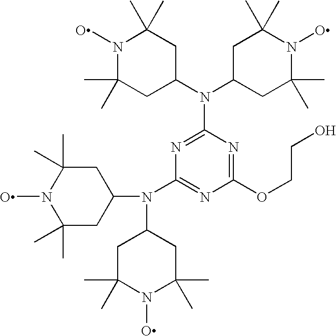

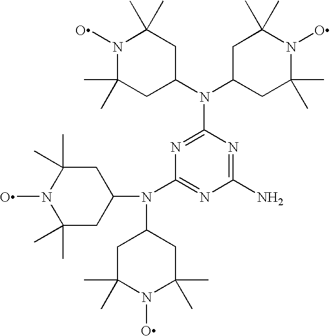

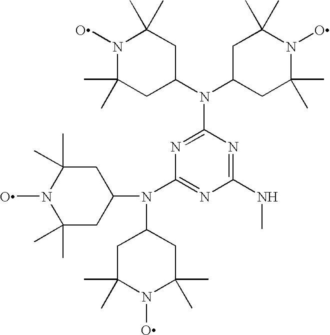

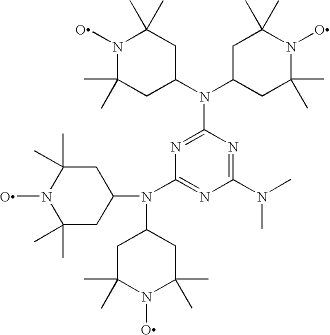

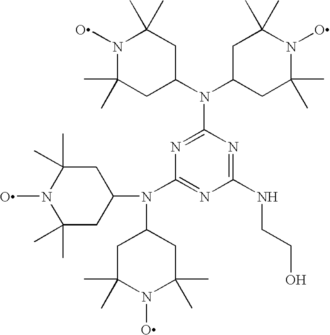

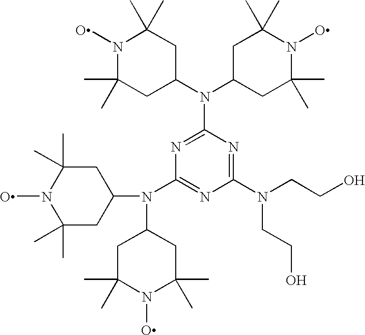

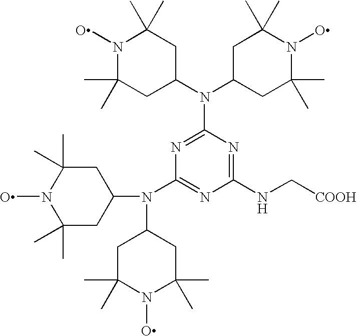

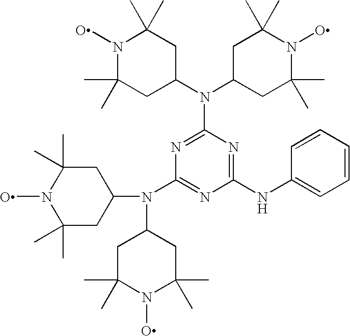

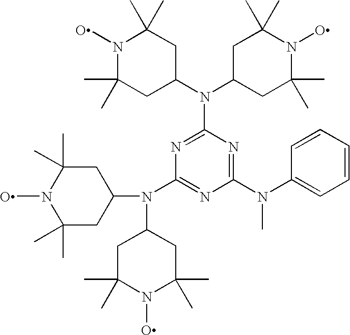

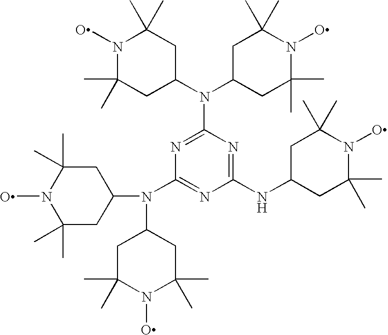

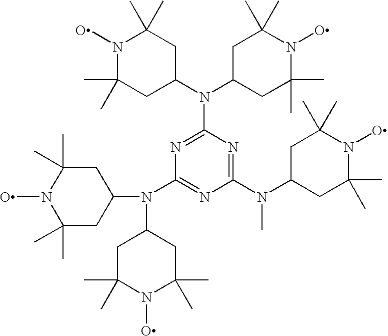

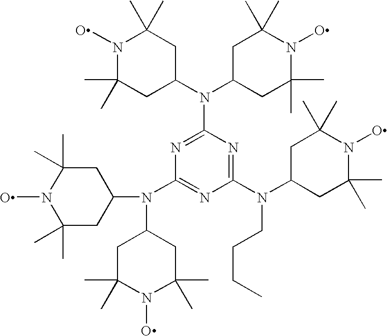

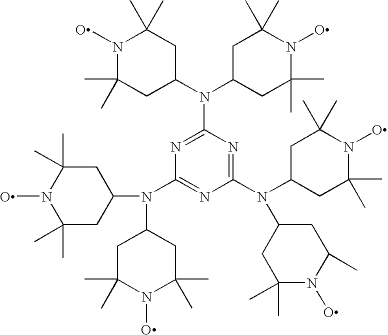

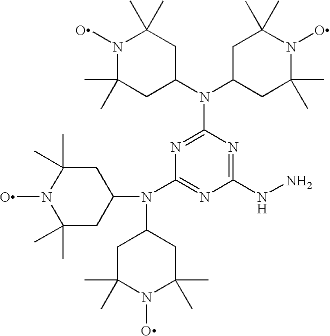

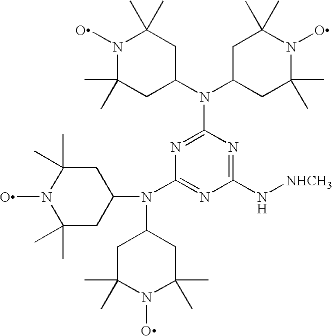

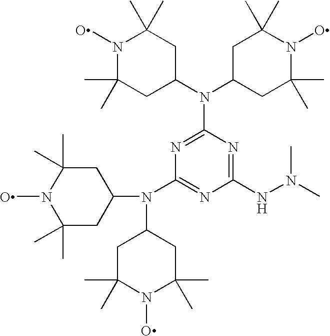

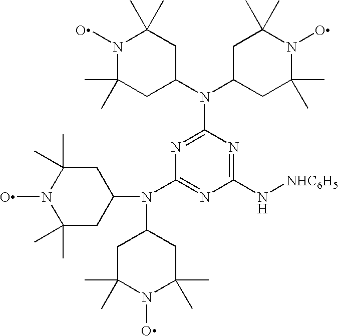

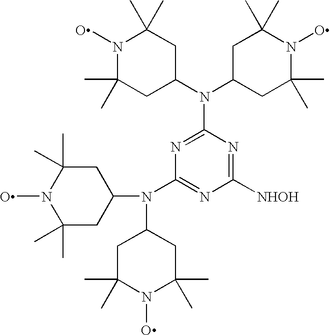

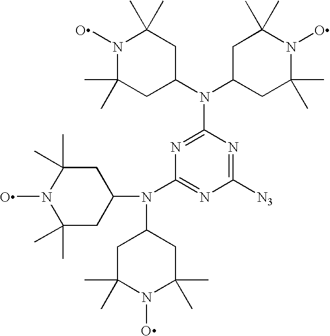

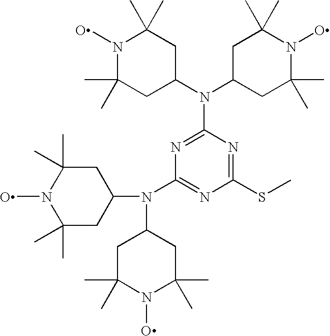

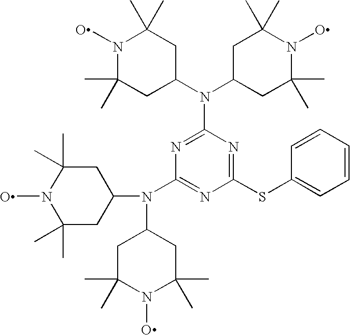

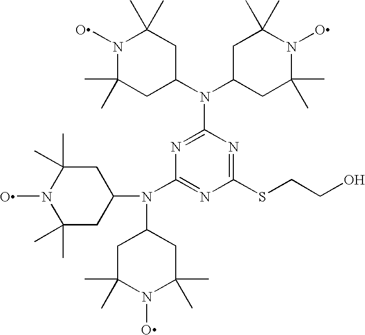

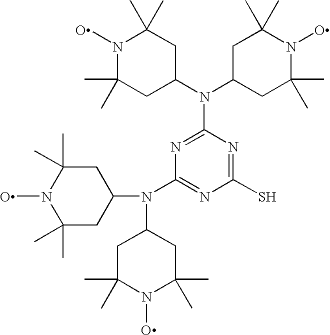

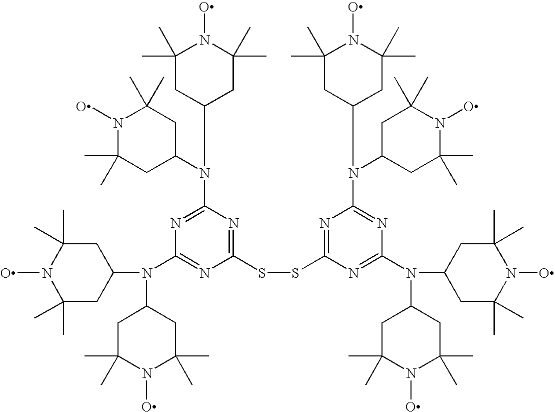

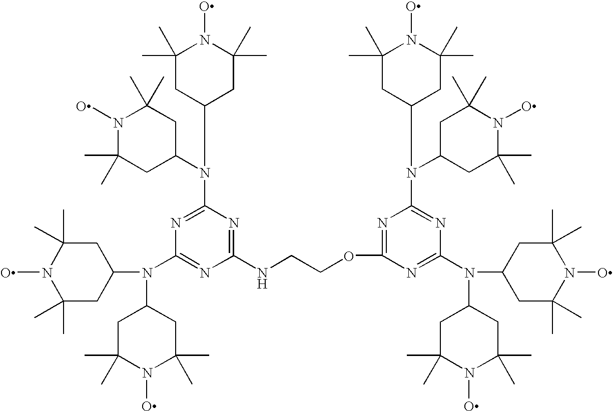

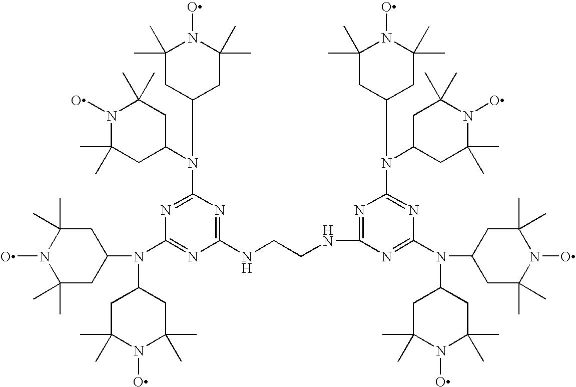

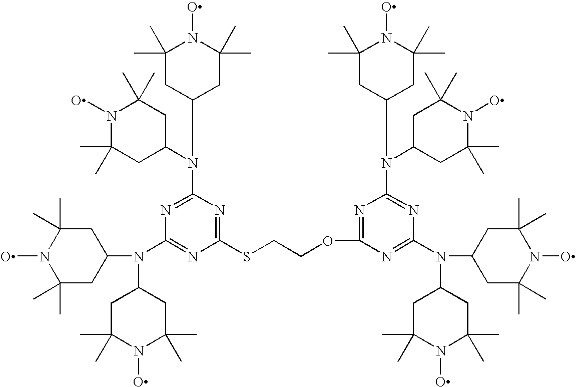

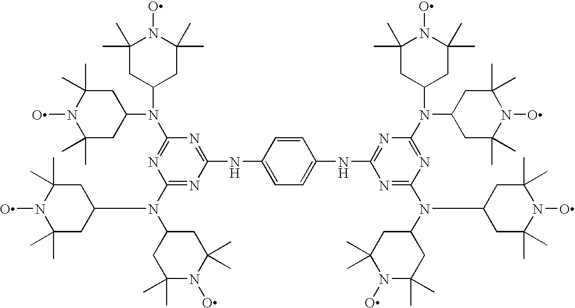

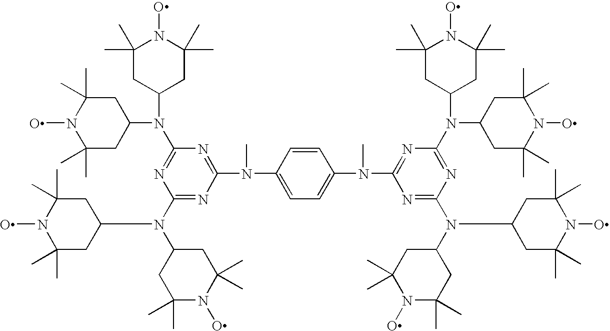

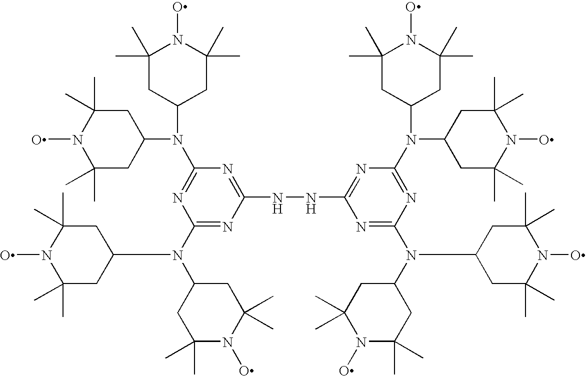

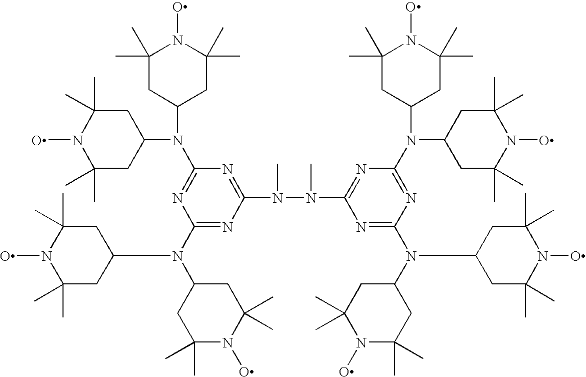

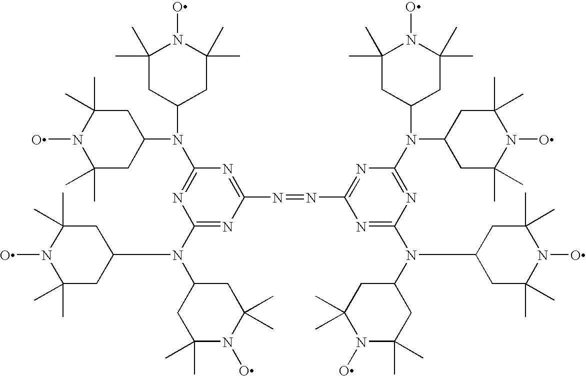

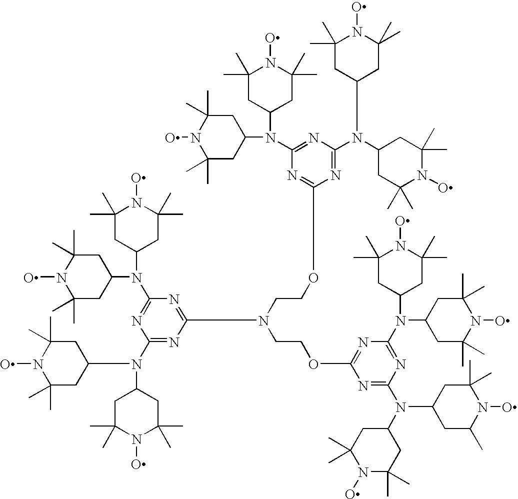

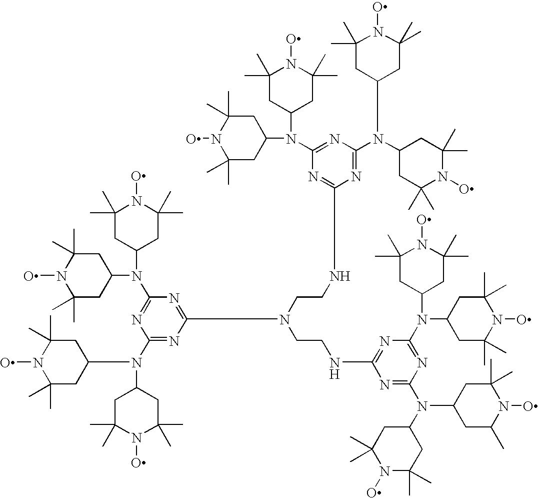

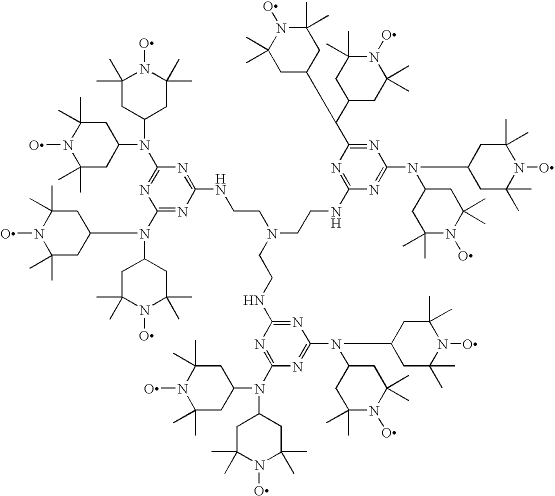

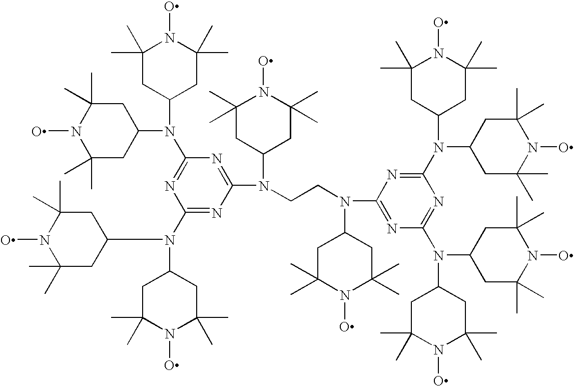

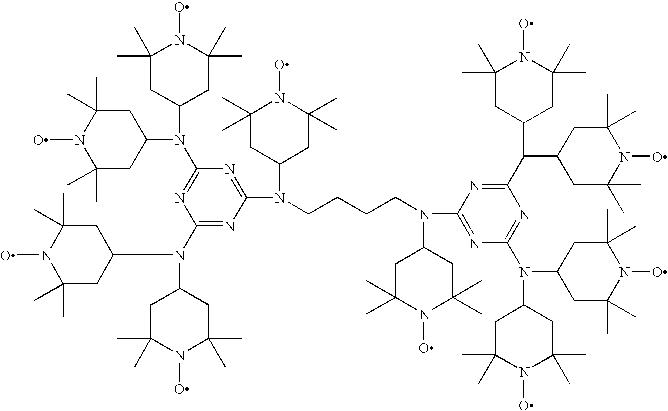

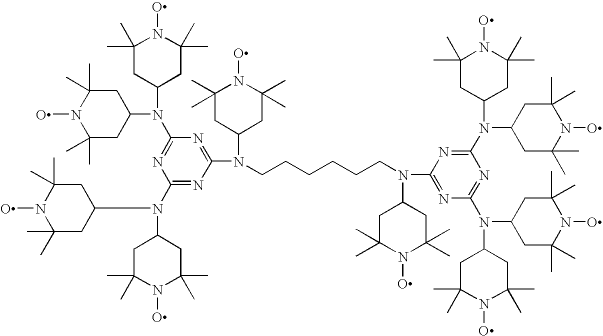

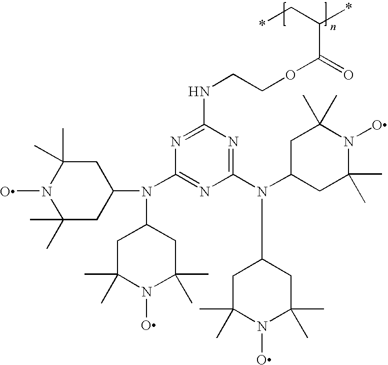

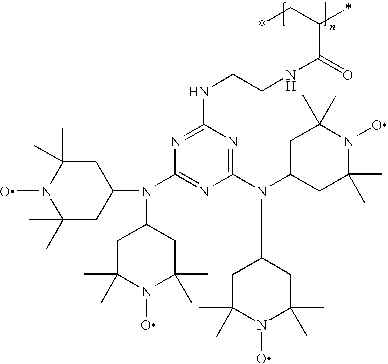

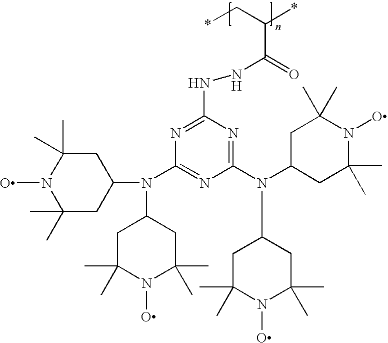

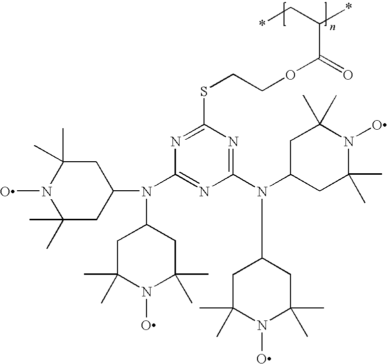

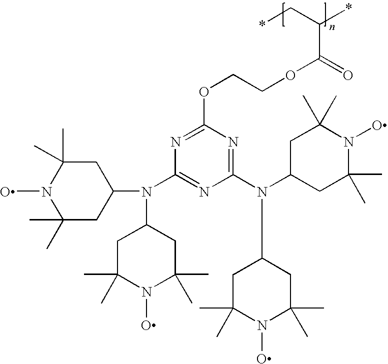

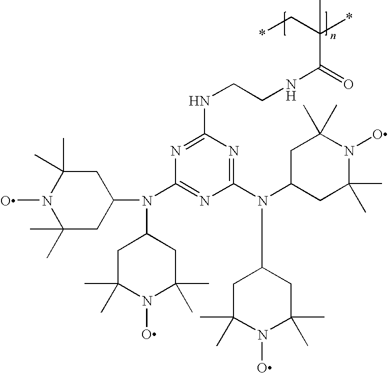

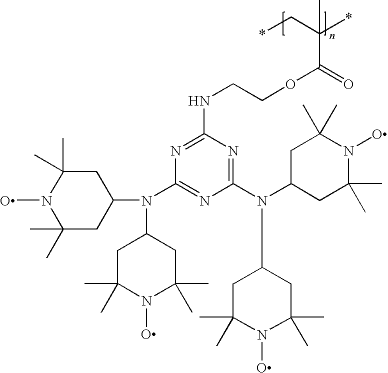

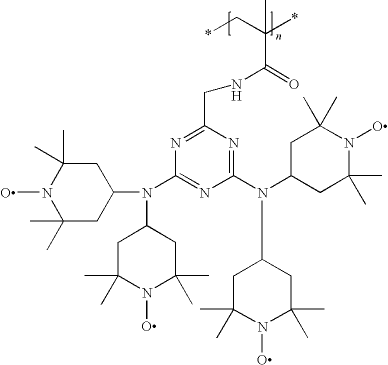

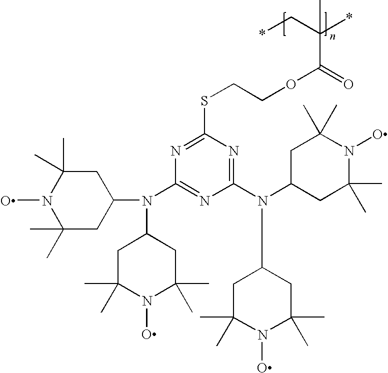

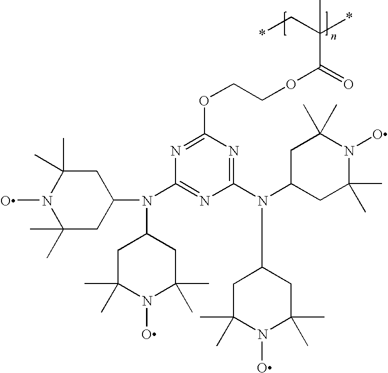

Spiro

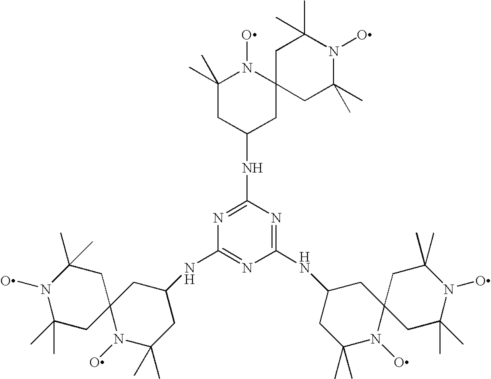

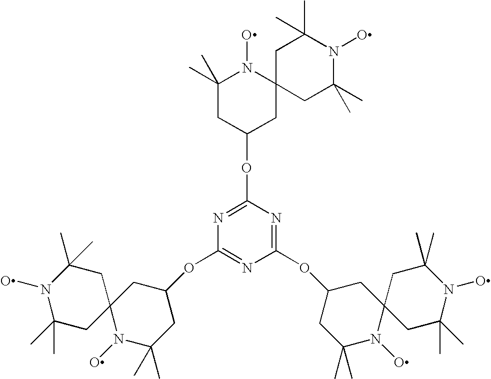

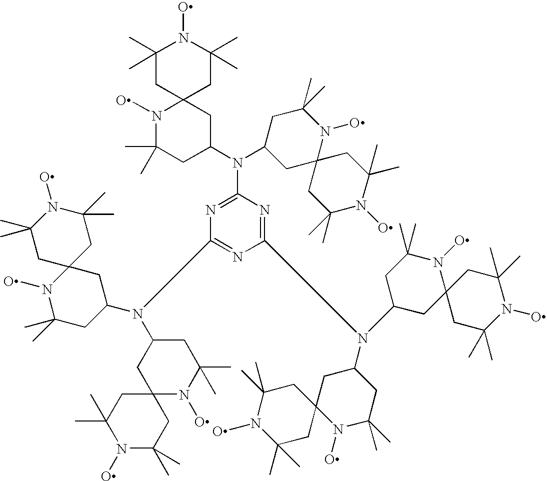

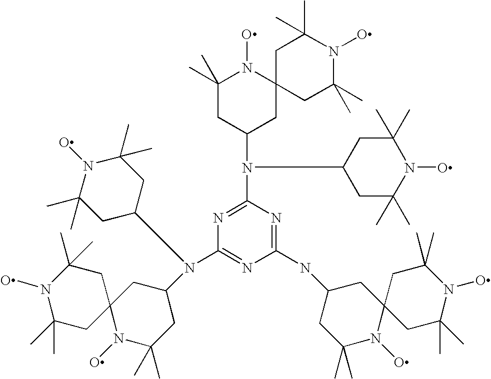

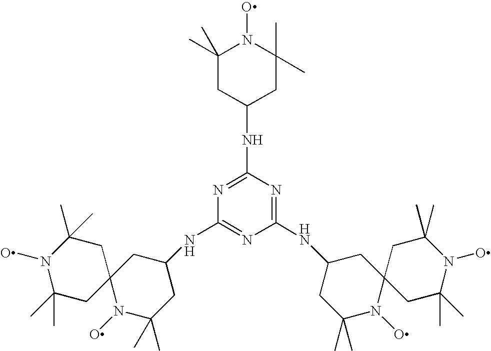

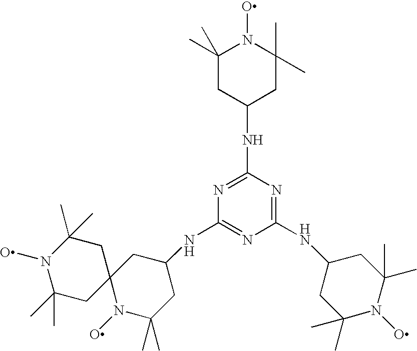

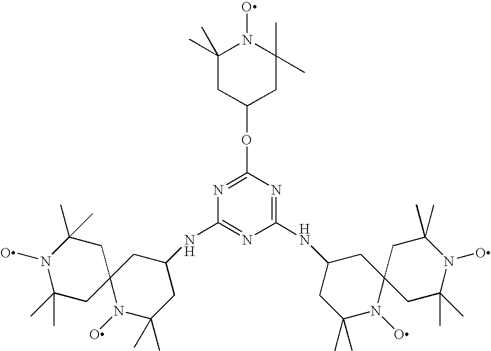

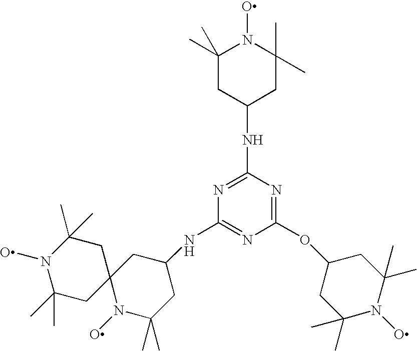

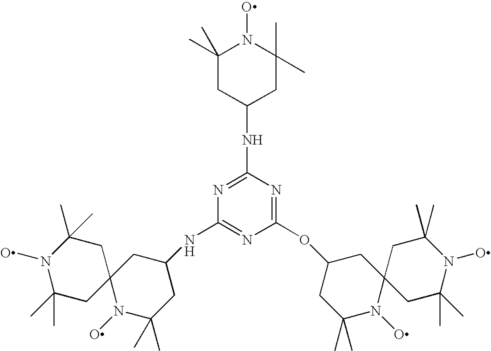

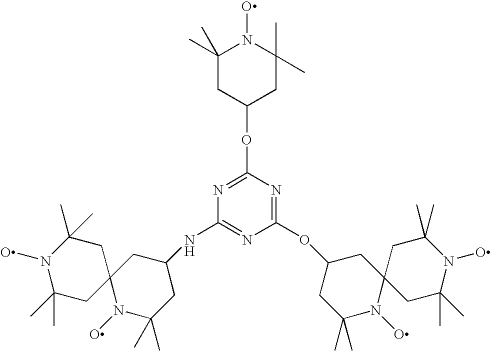

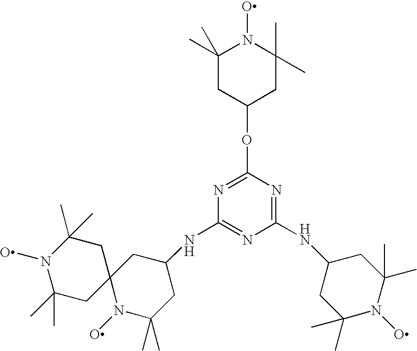

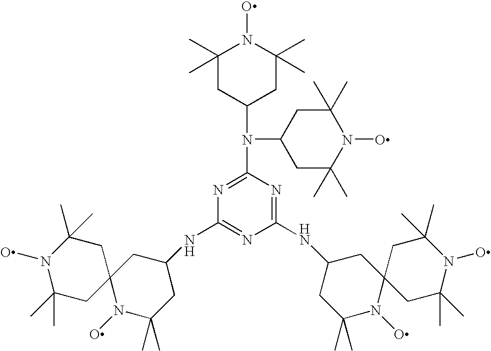

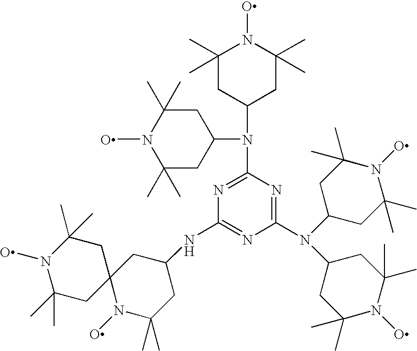

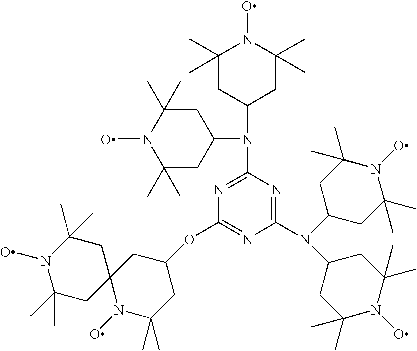

Other aspects of the invention are a method for providing a secondary battery, which method comprises incorporating an active material as defined above in at least one of the positive or negative electrodes; and

the use of a compound selected from the group consisting of radicals of formulae (Ia) to (Id), which are chemically bonded directly or via a linker group to a triazine structural element of formula (II)

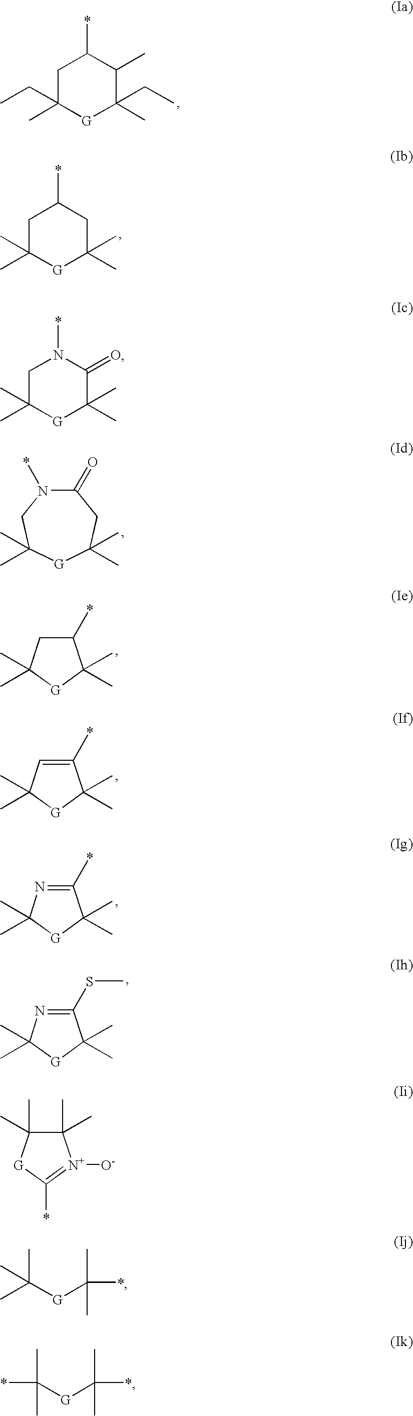

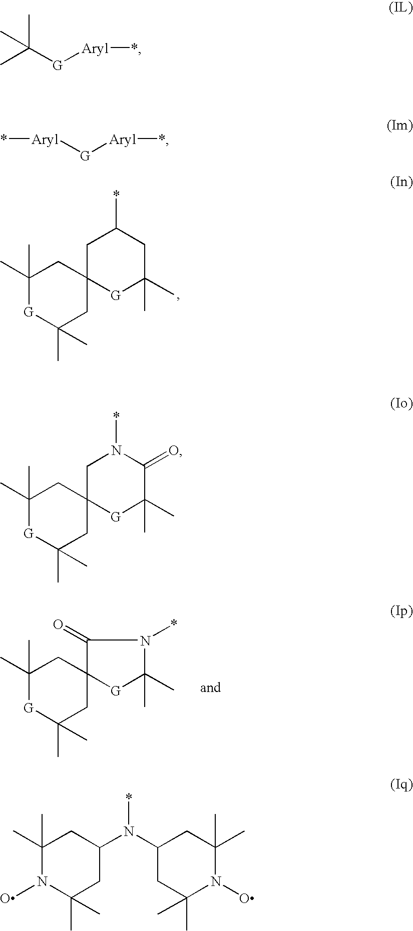

wherein
* is a valence indicating the bond to the triazine structural element;
Aryl is phenyl or naphthyl and

### G is

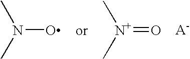

wherein A⁻ is an anion derived from an organic or inorganic acid;

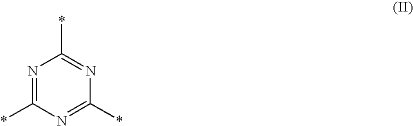

wherein
* is indicating a bond to which at least one radical of formulae (Ia) to (Iq) is attached either directly or via a linking group;
with the proviso that compound (A) is excluded

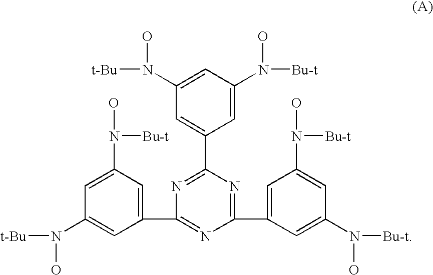

Yet a further aspect of the invention are novel nitroxyl radical compounds, which are particularly useful in the present invention.

For instance the compound is of formulae (c1) to (c7)

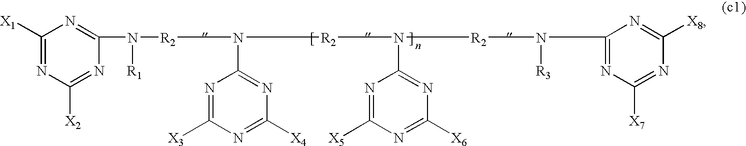

wherein
n is a number from 0-100;
R₁ and R₃ are independently H or C₁-C₁₂alkyl, C₅-C₇cycloalkyl, phenyl, C₁-C₁₂acyl or a group

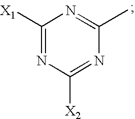

R₂ is C₂-C₁₂alkylene or phenylene;
X₁-X₈ independently of one another are groups of the formulas (c31)-(c45), preferably (c31) or (c32)

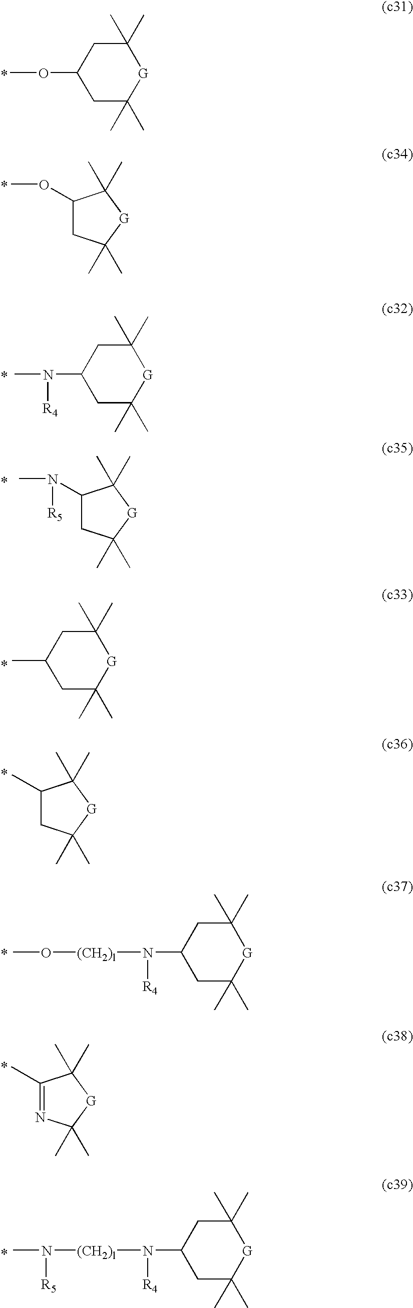

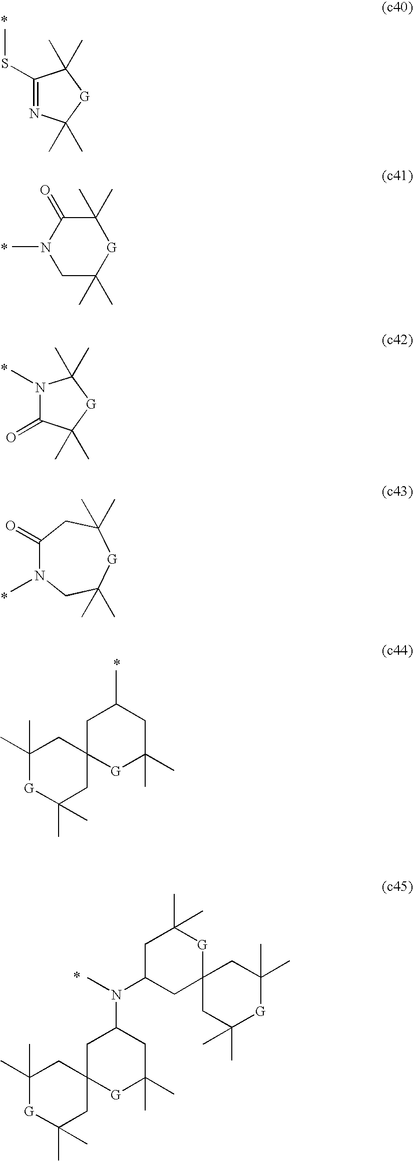

with the proviso that at least one group is of formula

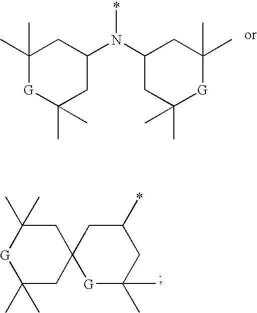

wherein

### I is 2-6;

R₄ and R₅ are hydrogen, C₁-C₁₂alkyl, C₅-C₆cycloalkyl, phenyl, C₁-C₁₂acyl or a group of formula (c33) or (c36);
additionally one or more of X₁-X₈ are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, NH₂, —NH—NH₂, —SH, C₁-C₁₂alkyl, phenyl, H, C₁-C₁₂alkenylamino, C₁-C₁₂alkynylamino, —SCN, —NHNH—C₁-C₁₂Alkyl, —NHNH-Phenyl, —N₃, C₅-C₆cycloalkyl, C₂-C₁₂alkenyl, C₂-C₁₂alkynyl, wherein the said alkyls are unsubstituted or substituted by OR₄, NR₄R₅ or SR₄, or the said alkyls comprise at least two carbon atoms and are uninterrupted or interrupted by —O—, —S— or —NR₄— or the said alkyl comprises at least two carbon atoms and is both substituted by OR₄, —NR₄R₅ or SR₄ and interrupted by —O—, —S— or —NR₄—; and

### G is

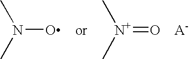

wherein A⁻ is an anion derived from an organic or inorganic acid;

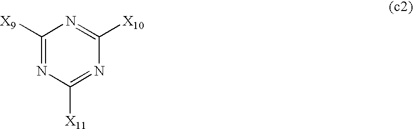

X₉-X₁₁ independently of one another are groups as defined for X₁-X₈;

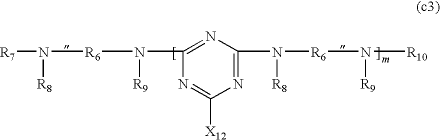

wherein
m is 0-100
R₆ is C₂-C₁₂alkylene, phenylene or a direct bond;
R₇, R₈, R₉ and R₁₀ independently are H or C₁-C₁₂alkyl, C₅-C₆cycloalkyl, phenyl, C₁-C₁₂acyl or the groups (c33) or (c36) or a group

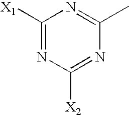

and X₁₂ has the meaning as defined for X₁-X₈;

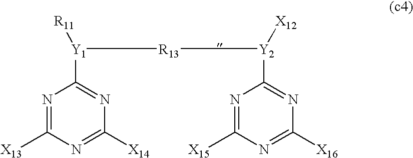

wherein

### Y₁ and Y₂ are N, O or S

R₁₃ is C₂-C₁₂alkylene, phenylene or a direct bond if Y₁ and Y₂ are N or S;
If Y₁ or Y₂ are O or S, then R₁₁ or R₁₂ are an electron pair;
If Y₁ and Y₂ are N, then R₁₁ and R₁₂ independently are H or C₁-C₁₂alkyl, C₅-C₆cycloalkyl, C₁-C₁₂acyl, phenyl or a group of formula (c33) or (c36) or a group

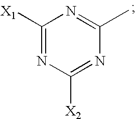

X₁₃-X₁₆ are independently as defined for X₁-X₈;

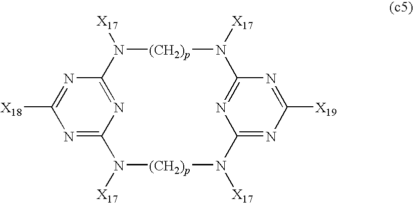

wherein
p is a number 0-18
X₁₇ is independently a group (c33) or (c36); and
X₁₈ and X₁₉ are independently groups as defined for X₁-X₈;

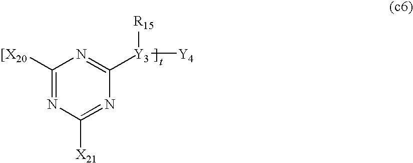

wherein
X₂₀ and X₂₁ are independently groups as defined for X₁-X₈;

### Y₃ is N, O or S;

If Y₃ is O or S, then R₁₅ is an electron pair;
If Y₃ is N, then R₁₅ is H or C₁-C₁₂alkyl, C₅-C₆cycloalkyl, C₁-C₁₂acyl, phenyl or a group of formula (c33) or (c36) or a group

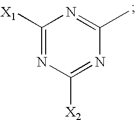

t is 3-6;
Y₄ is a trivalent-, tetravalent-, pentavalent or hexavalent inorganic or organic residue;

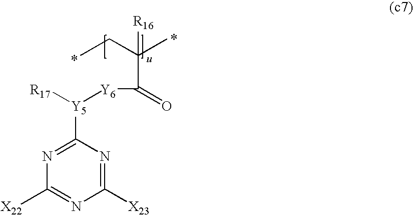

wherein

### R₁₆ is H or CH₃;

### Y₅ is N, O or S;

If Y₅ is O or S, then R₁₇ is an electron pair
If Y₅ is N, then R₁₇ is H or C₁-C₁₂alkyl, C₅-C₆cycloalkyl, C₁-C₁₂acyl, phenyl or a group of formula (c33) or (c36) or a group

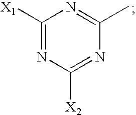

Y₆ is a direct bond, —NH— or —CH₂CH₂—N— or —CH₂CH₂—O—; and
X₂₂ and X₂₃ have the same meaning as defined for X₁-X₈.

For instance, X₁-X₈ independently of one another are groups of the formulas (c31)-(c45), preferably (c31) or (c32);

additionally one or more of X₁-X₈ are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, NH₂, —NH—NH₂, —SH, C₁-C₁₂alkyl or phenyl.

Compounds of formula (c2) are preferred.

In particular the compound is of formulae (d1) to (d7)

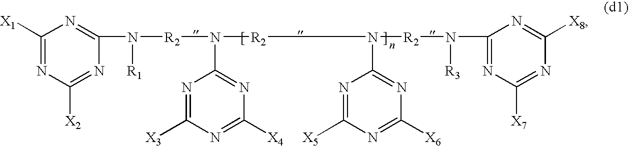

wherein
n is a number from 0-50
R₁ and R₃ are independently H, methyl, formyl, acetyl or a group

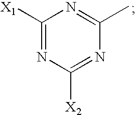

R₂ is C₂−C₆alkylene;
X₁-X₈ independently of one another are groups of the formulas (d31)-(d34), preferably (d31) or (d32),

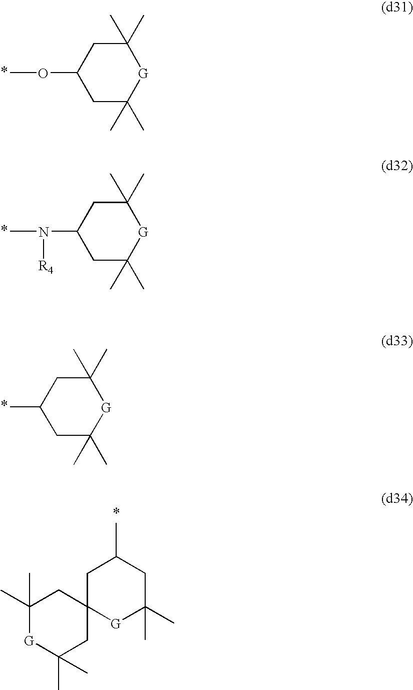

with the proviso that at least one is a group

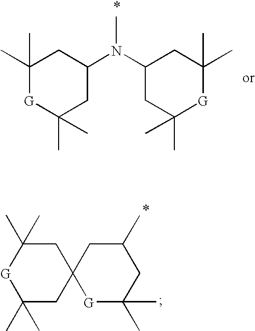

additionally one or more of X₁-X₈ are C₁-C₁₂alkyloxy, C₁-C₁₂alkylamino, C₁-C₁₂dialkylamino, carboxymethyl, halogen, —OH, NH₂, —SH, C₁-C₁₂alkyl H, C₁-C₁₂alkenylamino or C₁-C₁₂alkynylamino;

### G is

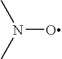

R₄ is hydrogen, C₁-C₄alkyl or a group of formula (d33);

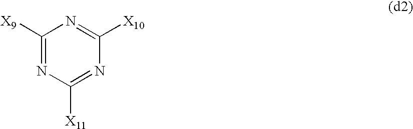

X₉-X₁₁ independently of one another are as defined for X₁-X₈;

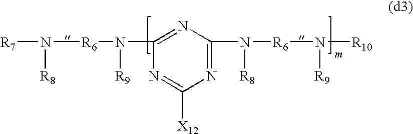

wherein
m is 0-50
R₆ is C₂-C₆alkylene or a direct bond;
R₇, R₈, R₉ and R₁₀ independently are H, methyl, formyl, acetyl or a group

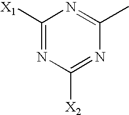

and X₁₂ has the meaning as defined for X₁-X₈;

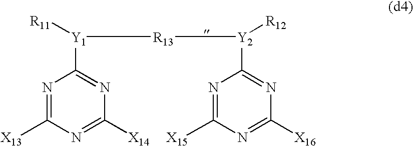

wherein

### Y₁ and Y₂ are N, O or S

R₁₃ is C₂-C₆alkylene or a direct bond if Y₁ and Y₂ are N or S;
If Y₁ or Y₂ are O or S, then R₁₁ or R₁₂ are an electron pair;
If Y₁ and Y₂ are N, then R₁₁ and R₁₂ independently are H, C₁-C₄alkyl or a group of formula (d33) or a group

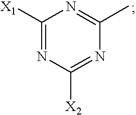

X₁₃-X₁₆ are independently as defined for X₁-X₈;

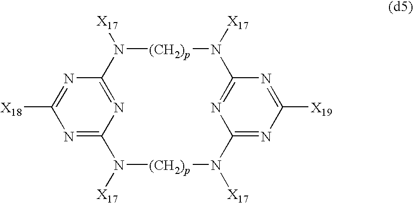

wherein
p is a number 2-6
X₁₇ is a group (d33); and
X₁₈ and X₁₉ are independently as defined for X₁-X₈;

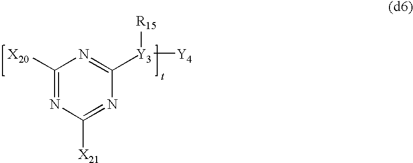

wherein
X₂₀ and X₂₁ are independently as defined for X₁-X₈,

### Y₃ is N, O or S;

If Y₃ is O or S, then R₁₅ is an electron pair;
If Y₃ is N, then R₁₅ is H, C₁-C₄alkyl or a group of formula (d33);
t is 3;
Y₄ is a trivalent inorganic or organic residue;

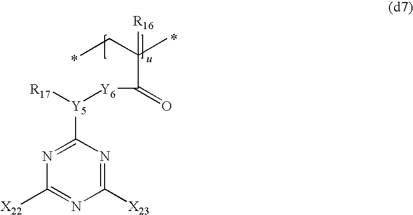

wherein

### R₁₆ is H or CH₃;

### Y₅ is N, O or S;

If Y₅ is O or S, then R₁₇ is an electron pair
If Y₅ is N, then R₁₇ is H or a group of formula (d33);
Y₆ is a direct bond, —NH— or —CH₂CH₂—N— or —CH₂CH₂—O—; and
X₂₂ and X₂₃ have the same meaning as defined for X₁-X₈.

Compounds of formula (d2) are preferred.

Preferably the compound is of formulae (e1) to (e7)

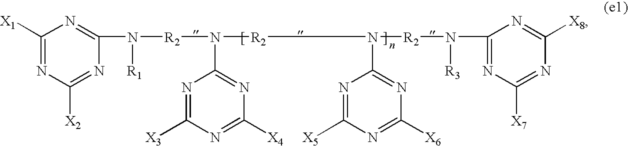

wherein
n is a number from 0-10
R₁ and R₃ are independently H or a group

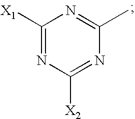

### R₂ is —CH₂—CH₂—;

X₁-X₈ independently of one another are groups of the formulas (e31)-(e34), preferably (e31) or (e32),

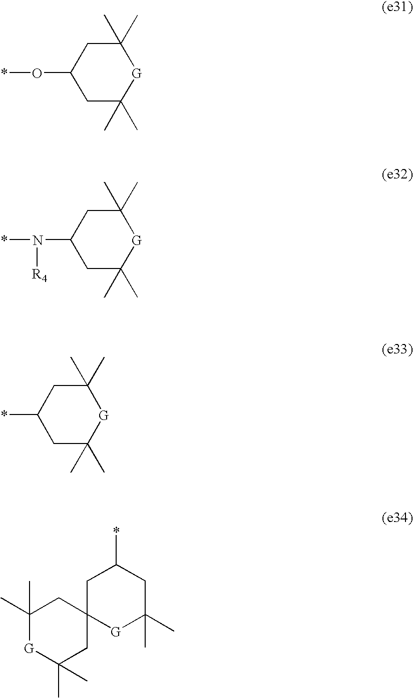

with the proviso that at least one is a group of formula

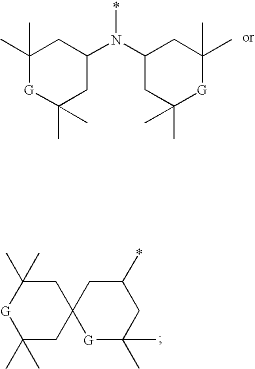

additionally one or more of X₁-X₈ are C₁-C₁₂alkylamino, C₁-C₁₂dialkylamino, halogen, NH₂, C₁-C₁₂alkenylamino or C₁-C₁₂alkynylamino;

### G is

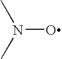

R₄ is hydrogen or a group of formula (e33);

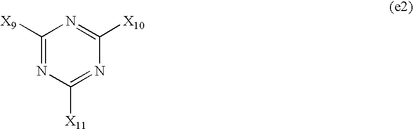

X₉-X₁₁ independently of one another are as defined for X₁-X₈;

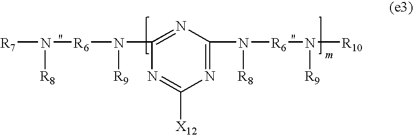

wherein
m is 0-10
R₆ is —CH₂CH₂— or a direct bond;
R₇, R₈, R₉ and R₁₀ are a group

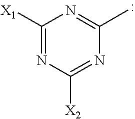

and X₁₂ has the meaning as defined for X₁-X₈;

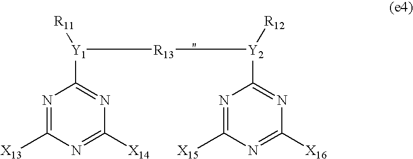

wherein

### Y₁ and Y₂ are N, O or S

R₁₃ is —CH₂CH₂— or a direct bond if Y₁ and Y₂ are N or S;
If Y₁ or Y₂ are O or S, then R₁₁ or R₁₂ are an electron pair;
If Y₁ and Y₂ are N, then R₁₁ and R₁₂ are a group of formula (e33) or a group

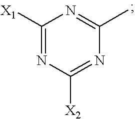

X₁₃-X₁₆ are independently as defined for X₁-X₈;

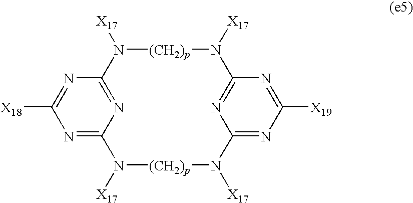

wherein
p is a number 2-6
X₁₇ is a group (e33); and X₁₈ and X₁₉ are independently as defined for X₁-X₈;

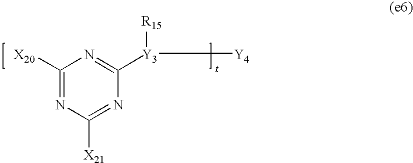

wherein
X₂₀ and X₂₁ are independently as defined for X₁-X₈, Y₃ is N, O or S;
If Y₃ is O or S, then R₁₅ is an electron pair;
If Y₃ is N, then R₁₅ is H or a group of formula (e33);
t is 3;
Y₄ is a trivalent inorganic or organic residue;

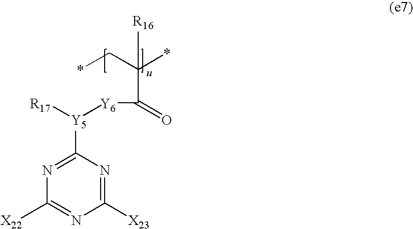

wherein

### R₁₆ is H;

### Y₅ is N or O;

If Y₅ is O, then R₁₇ is an electron pair
If Y₅ is N, then R₁₇ is H or a group of formula (e33);
Y₆ is a direct bond, —NH—, —CH₂CH₂—N— or —CH₂CH₂—O—; and
X₂₂ and X₂₃ have the same meaning as defined for X₁-X₈.

For instance, X₁-X₈ are as defined above.

For example, X₁-X₈ are independently of one another groups of formula (e31) or (e32); additionally one or more of X₁-X₈ are C₁-C₁₂alkylamino, C₁-C₁₂dialkylamino, halogen, NH₂, C₁-C₁₂alkenylamino or C₁-C₁₂alkynylamino.

For instance, X₁-X₈ are independently of one another groups of formula (e31) or (e32); additionally one or more of X₁-X₈ are C₁-C₁₂alkylamino, halogen, NH₂ or C₁-C₁₂alkynylamino.

For instance, the active material is of formula (e2).

The active material is preferably a compound of formula (e2) wherein

X₉, X₁₀, and X₁₁ are independently of one another groups of formula (e31) or (e32);
additionally one or more of X₉-X₁₁ are C₁-C₁₂alkylamino, C₁-C₁₂dialkylamino, halogen, NH₂,
C₁-C₁₂alkenylamino or C₁-C₁₂alkynylamino; and
R₄ is H or a group of formula (e33).

More preferably the active material is a compound of formula (e2) wherein

X₉, X₁₀, and X₁₁ are independently of one another groups of formula (e31) or (e32);
additionally one or more of X₉-X₁₁ are C₁-C₁₂alkylamino, halogen, NH₂ or C₁-C₁₂alkynylamino; and
R₄ is H or a group of formula (e33).

The definitions and preferences given above apply for all aspects of the invention.

The following examples illustrate the invention.

### A) PREPARATION EXAMPLES

### Example A1

### N,N′,N″-Tris-(2,2,6,6-tetramethyl-piperidin-4-yl-N-oxyl)-1,3,5-triazine-2,4,6-tri-amine (Cmpd. 101)

### A) N,N′,N″-Tris-(2,2,6,6-tetramethyl-piperidin-4-yl)-1,3,5-triazine-2,4,6-triamine

A 1500 ml four neck flask equipped with stirrer, thermometer and reflux condenser is charged with 600 ml 1,2-dichlorobenzene and 125 g (0.8 mol) 4-amino-2,2,6,6-tetramethylpiperidine. Then, 36.9 g (0.2 mol) cyanuric chloride are added at once and the mixture is stirred for 30 minutes. The resulting suspension is then stirred at reflux during 5 h and then cooled to room temperature. The solution of 25.2 g (0.63 mol) NaOH in 400 ml water is then added and the mixture is stirred 45 minutes at 80° C. It is then diluted with 500 ml hexane, cooled to 5° C. and filtered. The filter cake is dried, re-suspended in 300 ml hexane, filtered washed with hexane and dried to afford 88.4 g of the title compound as a white powder, mp. 219-221° C.

### B) Oxidation

A 2500 ml four neck flask equipped with stirrer, thermometer and dropping funnel is charged with 400 ml dichloromethane, 150 ml water, 113.4 g (1.35 mol) NaHCO₃, and 81.6 g (0.15 mol) N,N′,N″-Tris-(2,2,6,6-tetramethyl-piperidin-4-yl)-1,3,5-triazine-2,4,6-triamine. Peracetic acid (137 g, as 40% solution in acetic acid, 0.72 mol) is then added to the stirred mixture during 1 h while keeping the temperature between 25-32° C. Additional 150 ml water and 100 ml dichloromethane are added and the thick, red mixture is stirred 15 h at room temperature. Further 34 g peracetic acid (as 40% solution in acetic acid, 0.18 mol) are added and the mixture is stirred 5 h at room temperature. The red organic layer is then separated, washed with aqueous 5% Na₂CO₃ and water (100 ml each), dried over MgSO₄ and diluted with 250 ml methanol. The dichloromethane is then distilled off and the precipitated red crystals are filtered off. Further purification is performed by 4 times dissolving the crystals in ˜100 ml dichloromethane, adding ˜200 ml methanol and distilling off the dichloromethane. At the end, 31.8 g of the title material (Cmpd. 101) are obtained as a red, microcrystalline powder, mp 249-251° C. Elemental analysis for (C₃₀H₅₄N₉O₃) (588.82); calcd./found: C, 61.20/60.88; H, 9.24/9.22; N, 21.41/21.38. HPLC-MS: M=588.4.

### Example A2

### 1,3,5-Triazine, 2,4,6-tris[(2,2,6,6-tetramethyl-1-oxido-4-piperidinyl)oxy]- (Compd. 102).

This material is prepared as described in DE 2,319,816 (Example 7). Orange powder, mp. 201-3° C.

### Example A3

### N,N,N′,N′,N″-Pentakis-(2,2,6,6-tetramethyl-piperidin-4-yl-N-oxyl)-1,3,5-triazine-2,4,6-triamine (Cmpd. 103)

### A) 6-Chloro-N,N,N′,N′-tetrakis-(2,2,6,6-tetramethyl-piperidin-4-yl)-1,3,5-triazine-2,4-diamine

A 1500 ml four neck flask equipped with stirrer, thermometer and reflux condenser is charged with 350 ml 1,2-dichlorobenzene, 18.45 g (0.1 mol) cyanuric chloride and 65.0 g (0.22 mol) bis-(2,2,6,6-tetramethyl-piperidin-4-yl)-amine (prepared according to EP 838 455). The stirred mixture is refluxed during 285 minutes. It is then cooled to 120° C. and additional 53.2 g (0.18 mol) bis-(2,2,6,6-tetramethyl-piperidin-4-yl)-amine and 100 ml 1,2-dichlorobenzene are added. The mixture is then refluxed again during 150 minutes, cooled to room temperature and evaporated to dryness. To the residue are added 2800 ml dichloromethane and the solution of 8 g (0.2 mol) NaOH in 100 ml water. The organic layer is separated after 15 minutes stirring, washed with 200 ml water, dried over K₂CO₃ and evaporated. The solid residue is re-dispersed in 250 hexane, the solid is filtered off, washed with hexane and dried to afford 55.25 of the title compound as a white powder.

### B) N,N,N′,N′,N″-Pentakis-(2,2,6,6-tetramethyl-piperidin-4-yl)-1,3,5-triazine-2,4,6-triamine

A 100 ml three neck flask equipped with magnetic stirrer, thermometer and reflux condenser is charged with 25 ml 1,2-dichlorobenzene, 5.1 g (7.25 mmol) of the above described chloro-derivative and 2.3 g (14.7 mmol) 4-amino-2,2,6,6-tetramethylpiperidine. The mixture is refluxed during 4 h, then cooled to 5° C. and filtered. The solid is washed with hexane and dried. The off-white powder so obtained (3.16 g) is dispersed in a solution of 0.2 g NaOH in 40 ml water and heated to reflux during 30 minutes. Filtration after cooling, washing with water and drying affords 2.53 g of the title material as a white powder, mp 318-320° C.

### C) Oxidation

A 200 ml four neck flask equipped with stirrer, thermometer and dropping funnel is charged with 30 ml dichloromethane, 3.2 g (38 mmol) NaHCO₃, 5 ml water and 2.06 g (2.5 mmol) of the amine prepared as described above. To the stirred mixture is added during 5 minute 3.8 g (as 40% solution in acetic acid, 20 mmol) peracetic acid while keeping the temperature between 20-26° C. After 4 h stirring at room temperature additional 0.3 ml peracetic acid are added and the mixture is stirred for 24 h at room temperature. The organic layer is separated, washed with 1M Na₂CO₃ and water, dried over MgSO₄ and evaporated. The residue is chromatographically purified on silica gel with dichloromethane-ethylacetate (5:3) and then crystallized from ethyl acetate to afford 1.22 g of the title compound as red crystals, mp 260-262° C. HPLC-MS: single peak with M=896.7 (for C₄₈H₈₆N₁₁O₅ M=897.29)

### Example A4

### 1-Piperidinyloxy, 4,4′,4″, 4′″-[12,25-bis[(1,1,3,3-tetramethylbutyl)amino]-2,9,11,13,15,22,24,26,27,28-decaazatricyclo[21.3.1.110,14]octacosa-1(27),10,12,14(28),23,25-hexaene-2,9,15,22-tetrayl]tetrakis[2,2,6,6-tetramethyl- (Compd. 104).

This material is prepared as described in WO 02/058844 A1. Orange powder, mp. 267-270° C., nitroxyl content (by ESR) 95%.

### Example A5

### 6-Chloro-N,N,N′,N′-tetrakis-(2,2,6,6-tetramethyl-1-oxyl-piperidin-4-yl)-[1,3,5]triazine-2,4-diamine (Cmpd. 105)

A 1500 ml flask is charged with 35.1 g (0.05 mol) 6-chloro-N,N,N′,N′-tetrakis-(2,2,6,6-tetramethyl-piperidin-4-yl)-1,3,5-triazine-2,4-diamine (see example A3), 550 ml dichloromethane, 60 ml water and 50.4 g (0.6 mol) NaHCO₃. To the stirred mixture are during 70 minutes added 64.65 g (0.34 mol, 40% solution in acetic acid) of peracetic acid. The red mixture is stirred at room temperature for 18 h. The organic layer is then separated, washed with 5% aqueous Na₂CO₃, then 3× with water, dried over MgSO₄ and evaporated.

The residue is chromatographed on silica gel column with dichloromethane-ethyl acetate

(1:1) and then crystallized from methanol-dichloromethane to afford 26.1 g of the title compound. Red crystals, mp. 252-256° C. HPLC-MS: single peak with M=761.3 (for C₃₉H₆₈N₉O₄ M=762.48)

### Example A6

### N,N,N′,N′-Tetrakis-(2,2,6,6-tetramethyl-1-oxyl-piperidin-4-yl)-6-(2,2,6,6-tetramethyl-1-oxyl-piperidin-4-yloxy)-[1,3,5]triazine-2,4-diamine (Cmpd. 106)

A round bottom flask is charged with 3.8 g (22 mmol) 4-hydroxy-2,2,6,6-tetramethylpiperidin-N-oxyl, 0.96 g (22 mmol, 55% in parrafine) sodium hydride and 22 ml tetrahydrofurane. The mixture is stirred 2 h at 50° C. and then cooled to 28° C. Thereafter, 15.2 g (20 mmol) 6-chloro-N,N,N′,N′-tetrakis-(2,2,6,6-tetramethyl-1-oxyl-piperidin-4-yl)-[1,3,5]triazine-2,4-diamine Cmpd. 105) and 10 ml tetrahydrofurane are added, the mixture is refluxed 5 h and evaporated. The residue is chromatographed on silica gel column with dichloromethane-ethyl acetate (7:3) and then crystallized from methanol-dichloromethane to afford 15.2 g of the title compound. Red crystals, mp. 233-236° C. HPLC-MS: single peak with M=897.5 (for C₄₈H₈₅N10O₆ M=898.28).

### Example A7

### N,N,N′,N″-Tetrakis-(2,2,6,6-tetramethyl-1-oxyl-piperidin-4-yl)-[1,3,5]triazi-ne-2,4,6-triamine (Cmpd. 107)

N,N,N′,N″-Tetrakis-(2,2,6,6-tetramethyl-piperidin-4-yl)-[1,3,5]triazine-2,4,6-triamine

A 1500 ml flask is charged with 27.6 g (0.15 mol) cyanurchloride and 320 ml 1,2-dichlorobenzene. Thereafter, 44.45 g (0.15 mol) bis-(2,2,6,6-tetramethyl-piperidin-4-yl)-amine (prepared according to EP 838 455) are added during 20 minutes. The mixture is then during 7 h slowly heated from room temperature to 137° C. and thereafter kept 90 minutes at 137° C. Then, 70.3 g (0.45 mol) 4-amino-2,2,6,6-tetramethylpiperidine are added and the mixture is heated for 2 h at 180° C. The solvent is then evaporated and solution of 18 g (0.45 mol) NaOH in 150 ml water is added. The mixture is stirred 30 minutes at 90° C., then cooled, filtered and the solid is washed with water and dried. The dry material is suspended in 170 ml hexane, the suspension is stirred for 2 h, filtered and dried to afford 77.8 g of the title compound. White powder, mp. 235-243° C.

### B) Oxidation

A 1500 ml flask is charged with 35.1 g (0.051 mol) of the above prepared compound, 350 ml dichloromethane, 120 ml water and 48.2 g (0.574 mol) NaHCO₃. To the stirred mixture are during 70 minutes added 58.15 g (0.306 mol, 40% solution in acetic acid) of peracetic acid. The red mixture is stirred at room temperature for 19 h and then diluted with 50 ml water and 30 ml of 10% aqueous NaOH solution. The organic layer is then separated, washed with 1% aqueous NaOH, then 2× with water, dried over MgSO₄ and evaporated. The residue is chromatographed on silica gel column with dichloromethane-ethyl acetate (7:3) and then crystallized from dichloromethane to afford 17.0 g of the title compound. Red crystals, mp. 243-246° C. HPLC-MS: single peak with M=742.5 (for C₃₉H₇₀N₁₀O₄ M=743.06)

### Example A8

### N,N,N′,N′,N″,N″-Hexakis-(2,2,6,6-tetramethyl-1-oxyl-piperidin-4-yl)-[1,3,5]-triazine-2,4,6-triamine (Cmpd. 108)

### A) N,N,N′,N′,N″,N″-Hexakis-(2,2,6,6-tetramethyl-piperidin-4-yl)-[1,3,5]-triazine-2,4,6-tria-mine

A 50 ml steel autoclave is charged with 7.4 g (0.025 mol) bis-(2,2,6,6-tetramethyl-piperidin-4-yl)-amine (prepared according to EP 838 455) and 8.78 g (0.0125 mol) 6-chloro-N,N,N′,N′-tetrakis-(2,2,6,6-tetramethyl-piperidin-4-yl)-1,3,5-triazine-2,4-diamine (see example A3). The autoclave is then heated for 15 h at 250° C. The resulting solid is stirred 10 minutes with 500 ml dichloromethane, 75 ml water and 25 ml of 30% aqueous NaOH solution, the organic layer is separated, dried over MgSO₄ and evaporated. The residue is triturated with hexane, filtered and dried to afford 9.47 g of the title compound. White powder, mp. 325-330° C.

### B) Oxidation

A 100 ml flask is charged with 0.6 g (0.624 mmol) of the above prepared compound, 20 ml dichloromethane, 1.5 ml water and 1 g (11.9 mmol) NaHCO₃. To the stirred mixture are dropwise added 1.5 g (7.9 mmol, 40% solution in acetic acid) of peracetic acid. The red mixture is stirred at room temperature for 90 h and then diluted with 10 ml of 10% aqueous Na₂CO₃ solution. The organic layer is then separated, washed with water, dried over MgSO₄ and evaporated. The residue is chromatographed on silica gel column with dichloromethane-ethyl acetate-hexane (1:1:1) to afford 0.13 g of the title compound. Red crystals, mp. 258-261° C. HPLC-MS: single peak with M=1050.5 (for C₅₇H₁₀₂N₁₂O₆ M=1051.53).

### Example A9

### 6-Fluoro-N,N,N′,N′-tetrakis-(2,2,6,6-tetramethyl-1-oxyl-piperidin-4-yl)-[1,3,5]triazine-2,4-diamine (Cmpd. 109)

### A) 6-Fluoro-N,N,N′,N′-tetrakis-(2,2,6,6-tetramethyl-piperidin-4-yl)-[1,3,5]triazine-2,4-dia-mine

A 300 ml flask is charged with 150 ml 1,2-dichlorobenzene and 44.35 g (0.15 mol) bis-(2,2,6,6-tetramethyl-piperidin-4-yl)-amine (prepared according to EP 838 455). Cyanuric fluoride (6.75 g, 0.05 mol) is added dropwise and the mixture is then heated for 4.5 h at reflux. The solvent is thereafter evaporated and the residue is stirred for 30 minutes with the solution of 4 g NaOH in 50 ml water and 100 ml hexane The suspension is then filtered, washed with hexane and water and dried to afford 30 g of the title compound as a white powder, mp. 298-304° C.

### B) Oxidation

A 750 ml flask is charged with 13.7 g (20 mmol) of the above prepared compound, 250 ml dichloromethane. 25 ml water and 20.7 g (246 mmol) NaHCO₃. To the stirred mixture are dropwise added 26 g (137 mmol, 40% solution in acetic acid) of peracetic acid. The red mixture is stirred at room temperature for 3.5 h and then diluted with 75 ml of 2 M aqueous Na₂CO₃ solution. The organic layer is then separated, washed with water, dried over MgSO₄ and evaporated. The residue is chromatographed on silica gel column with dichloromethane-ethyl acetate (4:1) to afford 8.2 g of the title compound. Red crystals, mp. 254-257° C. HPLC-MS: single peak with M=745 (for C₃₉H₆₈N₉O₄ M=746.03).

### Example A10

### N,N,N′,N′-Tetrakis-(2,2,6,6-tetramethyl-1-oxyl-piperidin-4-yl)-[1,3,5]tria-zine-2,4,6-triamine (Cmpd. 110) N,N,N′,N′-Tetrakis-(2,2,6,6-tetramethyl-piperidin-4-yl)-[1,3,5]triazine-2,4,6-triamine

A 50 ml steel autoclave is charged with 14 g (0.02 mol) 6-chloro-N,N,N′,N′-tetrakis-(2,2,6,6-tetramethyl-piperidin-4-yl)-1,3,5-triazine-2,4-diamine (see example A3) and 11 g of a methanolic ammonia solution (16.6 weight %). The autoclave is then heated for 13 h at 200° C. The resulting solid is refluxed 1 h with 100 ml water, filtered, washed with water and dried to afford 11.9 of the title compound as a white solid.

### B) Oxidation

A 750 ml flask is charged with 9 g (13.2 mmol) of the above prepared compound and 400 ml dichloromethane. m-Chloroperbenzoic acid (20.8 g, 84.4 mmol. 70%) is then added during 1 h while keeping the temperature at 20-22° C. The mixture is then diluted with 90 ml of 2M aqueous Na₂CO₃ solution and the organic layer is separated, washed with water and evaporated. The residue is crystallized 2× from dichloromethane-methanol to afford 6.0 g of the title compound. Red crystals, mp. 249-253° C. HPLC-MS: single peak with M=742.5 (for C₃₉H₇₀N₁₀O₄ M=743.06).

### Example A11

### N-Methyl-N′,N′,N″,N″-tetrakis-(2,2,6,6-tetramethyl-1-oxyl-piperidin-4-yl)-[1,3,5]triazine-2,4,6-triamine (Cmpd. 111)

### A) N-Methyl-N′,N′,N″,N″-tetrakis-(2,2,6,6-tetramethyl-piperidin-4-yl)-[1,3,5]triazine-2,4,6-triamine

A 50 ml steel autoclave is charged with 14 g (0.02 mol) 6-chloro-N,N,N′,N′-tetrakis-(2,2,6,6-tetramethyl-piperidin-4-yl)-1,3,5-triazine-2,4-diamine (see example A3) and 12.5 ml of an ethanolic methyl amine solution (33 weight %). The autoclave is then heated for 16 h at 200° C. The resulting solid is stirred with 100 ml water, filtered, washed with water and dried to afford 12 g of the title compound as a white solid.

### B) Oxidation

A 750 ml flask is charged with 12 g (17.2 mmol) of the above prepared compound, 350 ml dichloromethane, 20 ml water and 21.4 g (0.255 mol) NaHCO₃. To the stirred mixture are dropwise added 26 g (137 mmol, 40% solution in acetic acid) of peracetic acid. The red mixture is stirred at room temperature for 16 h and then diluted with 50 ml of 10% aqueous Na₂CO₃ solution, 100 ml water and 100 ml dichloromethane. The organic layer is then separated, washed with water, dried over MgSO₄ and evaporated. The residue is chromatographed on silica gel column with dichloromethane-ethyl acetate (4:1) to afford 6.7 g of the title compound. Red crystals, mp. 254-257° C. HPLC-MS: single peak with M=757 (for C₄₀H₇₂N₁₀O₄ M=757.08).

### Example A12

### N-Prop-2-ynyl-N′,N′,N″,N″-tetrakis-(2,2,6,6-tetramethyl-1-oxyl-piperidin-4-yl)-[1,3,5]triazine-2,4,6-triamine (Cmpd. 112)

### A) N-Prop-2-ynyl-N′,N′,N″,N″-tetrakis-(2,2,6,6-tetramethyl-piperidin-4-yl)-[1,3,5]triazine-2,4,6-triamine

A 250 ml steel autoclave is charged with 21.1 g (0.03 mol) 6-chloro-N,N,N′,N′-tetrakis-(2,2,6,6-tetramethyl-piperidin-4-yl)-1,3,5-triazine-2,4-diamine (see example A3), 5.0 g (0.091 mol) propargylamine and 75 ml toluene. The autoclave is then heated for 40 h at 200° C. The resulting solid is triturated with the solution of 1.6 g NaOH in 100 ml water, filtered and dried to afford 20 g of a white solid, containing (GC-MS) 92% of the title compound and 8% of starting material.

### B) Oxidation

A 750 ml flask is charged with 11.5 g (˜14.7 mmol) of the above prepared compound, 200 ml dichloromethane, 35 ml water and 22 g (0.262 mol) NaHCO₃. To the stirred mixture are dropwise added 23 g (0.121 mol, 40% solution in acetic acid) of peracetic acid. The red mixture is stirred at room temperature for 18 h and then diluted with 10 ml of 10% aqueous NaOH solution. The organic layer is then separated, washed with water, dried over MgSO₄ and evaporated. The residue is chromatographed on silica gel column with dichloromethane-ethyl acetate (7:3) to afford 2.45 g of the title compound. Red crystals, mp. 249-252° C. HPLC-MS: M=780 (for C₄₂H₇₂N₁₀0₄ M=781.11).

The compounds illustrating the present invention are compiled in the following Table 1

TABLE 1

| Nr | Structure |
|---|---|
| 101 | 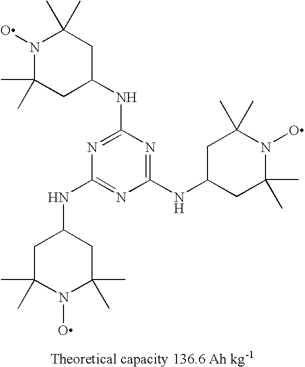 |
| 102 | 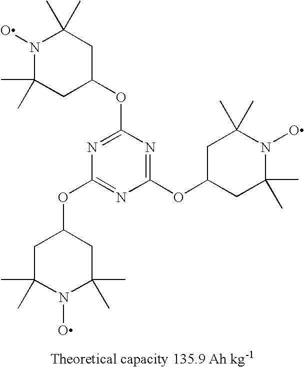 |
| 103 | 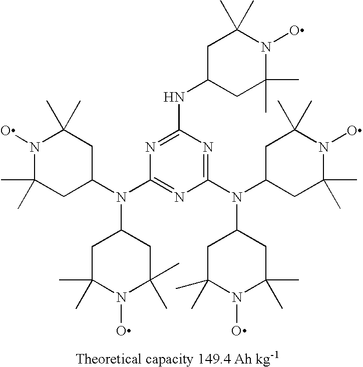 |
| 104 | 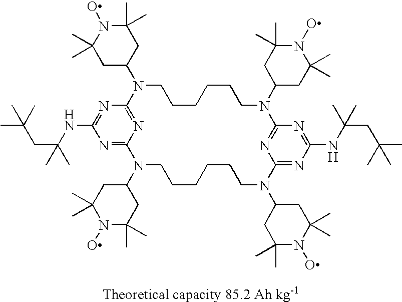 |
| 105 | 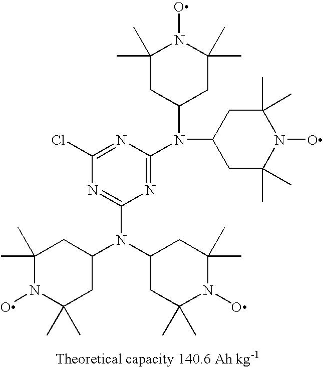 |
| 106 | 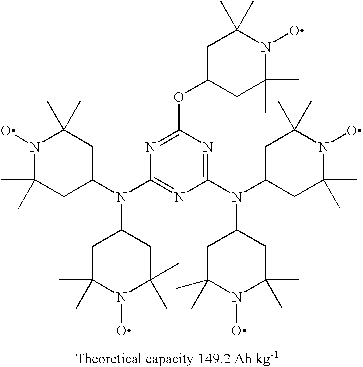 |
| 107 | 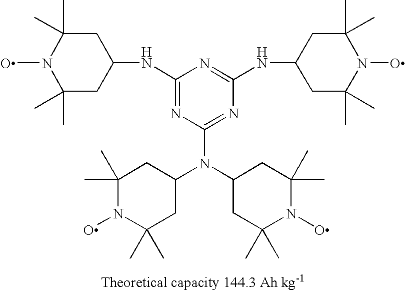 |
| 108 | 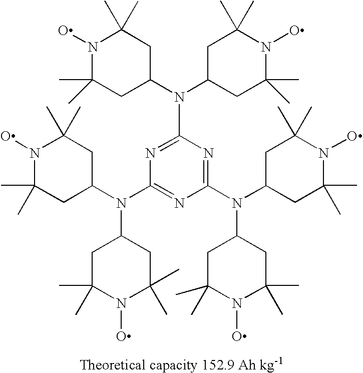 |
| 109 | 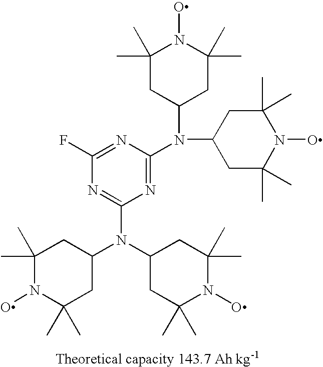 |
| 110 | 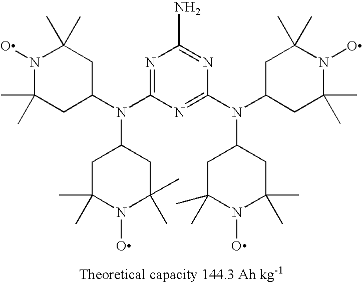 |
| 111 | 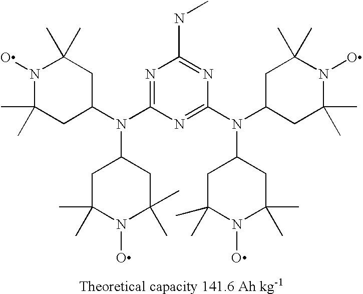 |
| 112 | 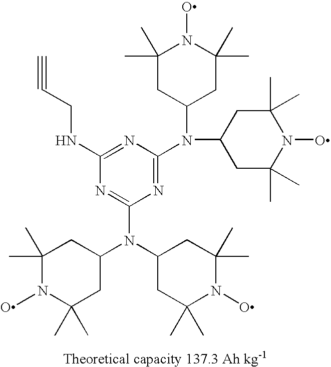 |

### B) APPLICATION EXAMPLES

### General Remarks

An important parameter of a successful electrode material consisting of nitroxide radicals is its charge capacity C [Ah/kg]. This is calculated according to eq 1:

C [Ah/kg]=n×F/3600=f×26803.64/M (Eq. 1)

n=Moles nitroxide/kg material;
F=Faraday constant=96 493.1 Coulomb;
M=Nitroxide molecular weight in g/Mol;
f=Number of nitroxide groups in the molecule.

This equation has been used to determine the theoretical charge capacity.

### Example B1

### Evaluation of Compound 101 in a Battery

One part of compound 101 is thoroughly mixed with 8 parts of vapor grown carbon fibers and 1 part of poly(tetrafluoroethylene) binder. The mixture is formed by roll press into a thin electrode from which a 12 mm diameter cathode is punched out. A coin cell consisting of Lithium metal anode, ethylene carbonate-diethyl carbonate (3/7 v/v) electrolyte containing 1M LiPF₆ and separator is then assembled. Repeated charging-discharging cycles indicated an average discharge capacity of 165 Ah/kg. This is 28.4 Ah/kg more then the calculated capacity (136.6 Ah/kg) for Compound 101 using equation (1).

### Example B2

### Evaluation of Compound 105 in a Battery

Cmpd. 105 is tested in analogy to Example B1

Repeated charging-discharging cycles indicated an average discharge capacity of 160 Ah/kg. This is 19.4 Ah/kg more then the calculated capacity (140.6 Ah/kg) for Compound 105 using equation (1).

1. A secondary battery with improved capacity, wherein an electrode reaction of an active material in the reversible oxidation/reduction cycle occurs in at least one of the positive or negative electrodes, which active material comprises a compound selected from the group consisting of radicals of formulae (Ia) to (Iq), which are chemically bonded directly or via a linking group to a triazine structural element of formula (II) 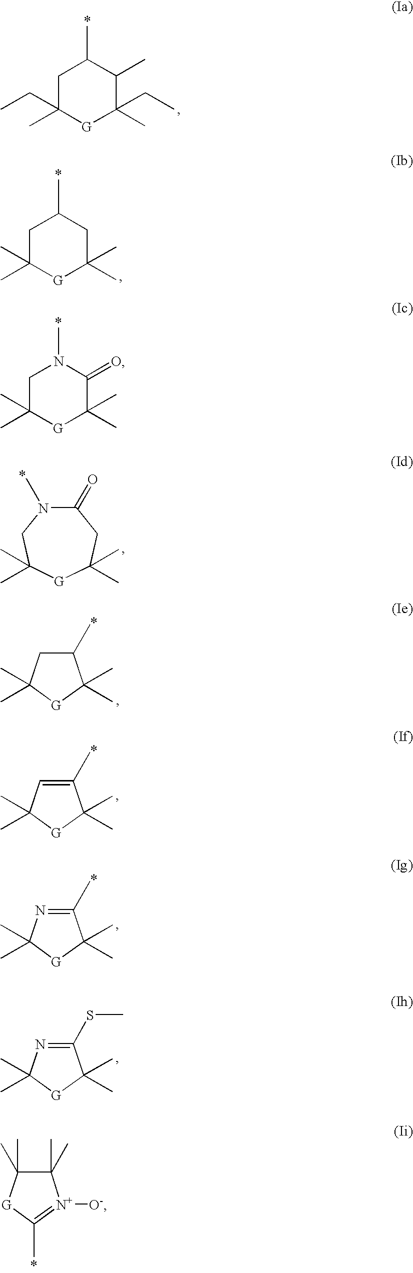 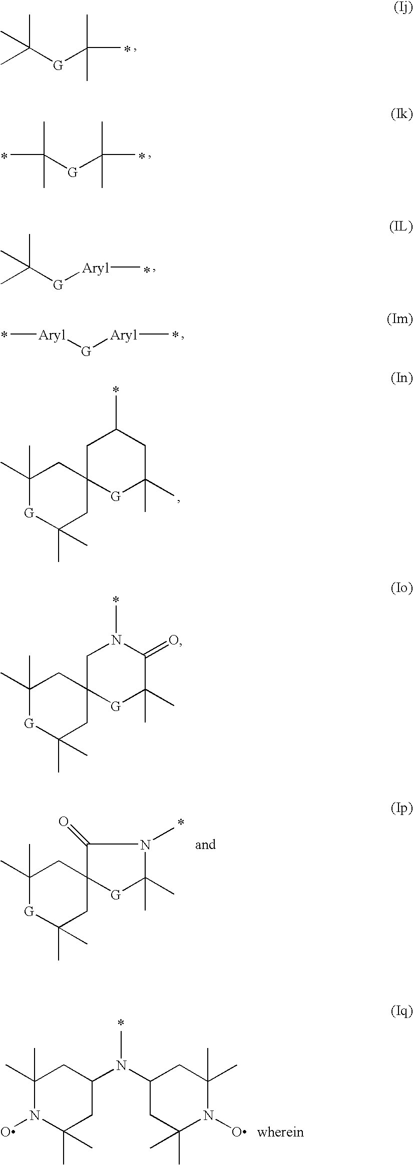
* is a valence indicating the cite of the bond to the triazine structural element;
Aryl is phenyl or naphthyl and
G is 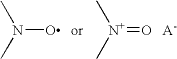
wherein A⁻ is an anion derived from an organic or inorganic acid; 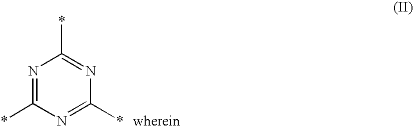
* indicates a bond to which at least one radical of formulae (Ia) to (Iq) is attached, either directly or via a linking group;
with the proviso that compound (A) is excluded

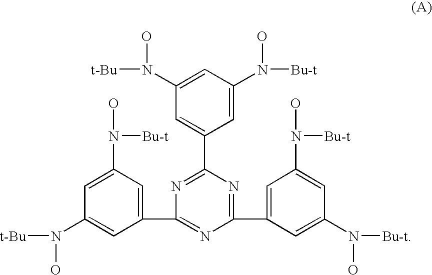

2. A secondary battery according to claim 1 wherein the electrode reaction of the active material occurs in the positive electrode.

3. A secondary battery according to claim 1 wherein the active material comprises from 10 to 100% by weight of the compound selected from the group consisting of radicals of formulae (Ia) to (Iq), which are chemically bonded to a triazine structural element of formula (II).

4. A secondary battery according to claim 1 wherein the active material has a spin concentration of at least 10²¹ spins/g.

5. A secondary battery according to claim 1 wherein G is a nitroxide radical

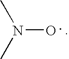

6. A secondary battery according to claim 1 wherein at least two radicals of formulae (Ia) to (Iq) are bonded to the triazine structural element of formula (II).

7. A secondary battery according to claim 1 wherein the triazine structural element of formula (II) is part of a repeating unit of an oligomer or polymer.

8. A secondary battery according to claim 1 wherein the radicals are of formulae (Ib), Ic), (In), (Ip) or (Iq).

9. A secondary battery according to claim 1 wherein the active material comprises a compound of formulae (c1) to (c7) 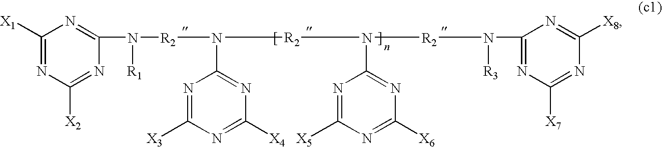
wherein
n is a number from 0-100;
R₁ and R₃ are independently H or C₁-C₁₂alkyl, C₅-C₇cycloalkyl, phenyl, C₁-C₁₂acyl or a group 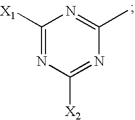
R₂ is C₂-C₁₂alkylene or phenylene;
X₁-X₈ independently of one another are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, —NH₂, —NH—NH₂, —SH, C₁-C₁₂alkyl, phenyl, H, C₁-C₁₂alkenylamino, C₁-C₁₂alkynylamino, —SCN, —NHNH—C₁-C₁₂Alkyl, —NHNH-Phenyl, —N₃, C₅-C₆cycloalkyl, C₂-C₁₂alkenyl, C₂-C₁₂alkynyl, wherein the said alkyls are unsubstituted or substituted by OR₄, NR₄R₅ or SR₄ or the said alkyls comprise at least two carbon atoms and are uninterrupted or interrupted by —O—, —S— or —NR₄— or the said alkyl comprises at least two carbon atoms and is both substituted by OR₄, —NR₄R₅ or SR₄ and interrupted by —O—, —S— or —NR₄—
or groups of the formulas (c31)-(c45), 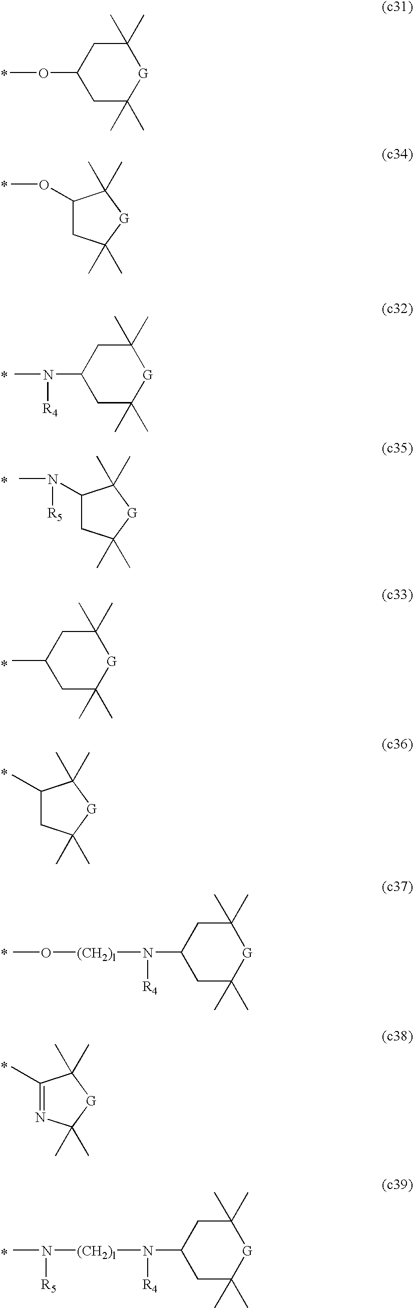 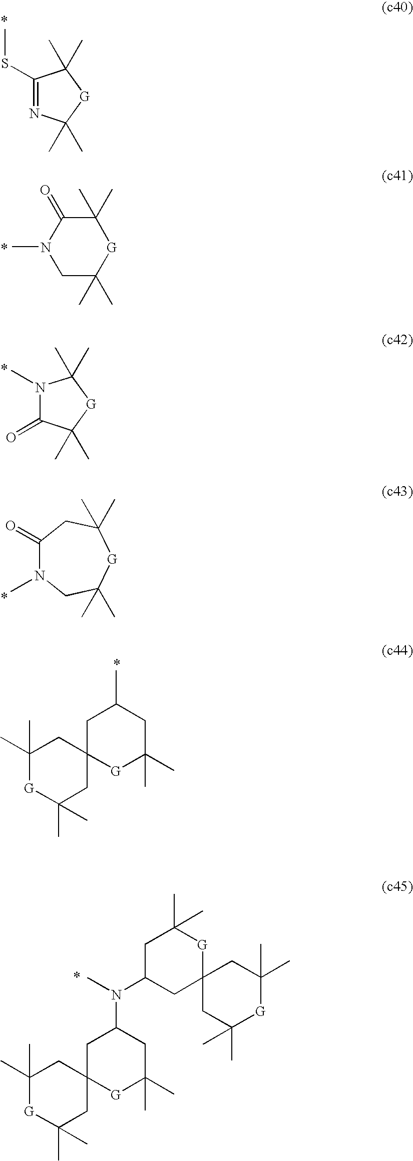
wherein
I is 2-6;
R₄ and R₅ are hydrogen, C₁-C₁₂alkyl, C₅-C₆cycloalkyl, phenyl, C₁-C₁₂acyl or a group of formula (c33), (c36) or (c44);
with the proviso that at least one of X₁ to X₈ is a group according to the formulas (c31)-(c45); 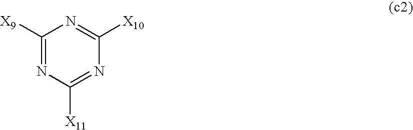
wherein X₉-X₁₁ independently of one another are groups as defined for X₁-X₈; 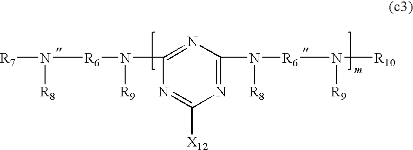
wherein
m is 0-100
R₆ is C₂-C₁₂alkylene, phenylene or a direct bond;
R₇, R₈, R₉ and R₁₀ independently are H or C₁-C₁₂alkyl, C₅-C₆cycloalkyl, phenyl, C₁-C₁₂acyl or the groups (c33) or (c36) or a group 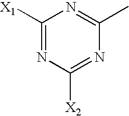
and X₁₂ has the meaning as defined for X₁-X₈; 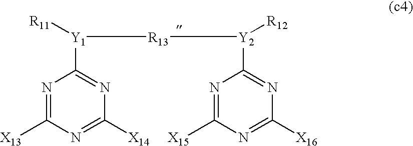
wherein
Y₁ and Y₂ are N, O or S
R₁₃ is C₂-C₁₂alkylene, phenylene, C₂-C₁₂ and when Y₁ and Y₂ are N or S R₁₃ may also be a direct bond;
If Y₁ or Y₂ are O or S, then R₁₁ or R₁₂ are an electron pair;
additionally, if Y₁ is N, then Y₂ and R₁₃ together can be direct bond;
If Y₁ and Y₂ are N, then R₁₁ and R₁₂ independently are H or C₁-C₁₂alkyl, C₅-C₆cycloalkyl, C₁-C₁₂acyl, phenyl or a group of formula (c33) or (c36) or a group 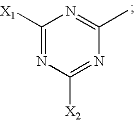
X₁₃-X₁₆ are independently as defined for X₁-X₈; 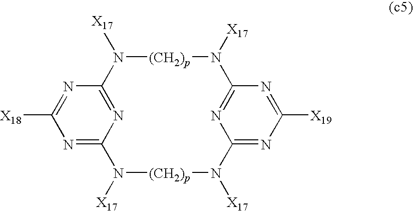
wherein
p is a number 0-18
X₁₇ is independently a group (c33) or (c36); and
X₁₈ and X₁₉ are independently groups as defined for X₁-X₈; 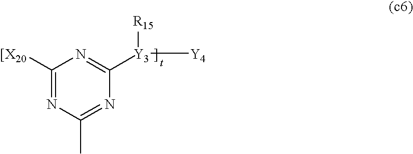
wherein
X₂₀ and X₂₁ are independently groups as defined for X₁-X₈;
Y₃ is N, O or S;
If Y₃ is O or S, then R₁₅ is an electron pair;
If Y₃ is N, then R₁₅ is H or C₁-C₁₂alkyl, C₅-C₆cycloalkyl, C₁-C₁₂acyl, phenyl or a group of formula (c33) or (c36) or a group 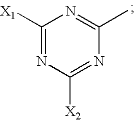
t is 3-6;
Y₄ is a trivalent-, tetravalent-, pentavalent or hexavalent inorganic or organic residue; 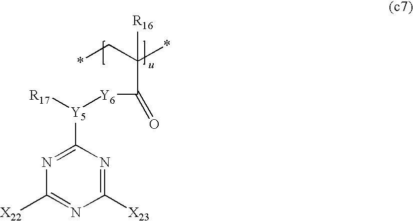
wherein
R₁₆ is H or CH₃;
Y₅ is N, O or S;
If Y₅ is O or S, then R₁₇ is an electron pair
If Y₅ is N, then R₁₇ is H or C₁-C₁₂alkyl, C₅-C₆cycloalkyl, C₁-C₁₂acyl, phenyl or a group of formula (c33) or (c36) or a group 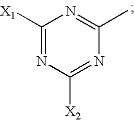
Y₆ is a direct bond, —NH— or —CH₂CH₂—N— or —CH₂CH₂—O—; and
X₂₂ and X₂₃ have the same meaning as defined for X₁-X₈.

10. A secondary battery according to claim 9 wherein the active material comprises a compound of formulae (d1) to (d7) 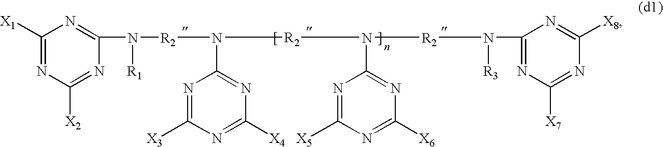
wherein
n is a number from 0-50
R₁ and R₃ are independently H, methyl, formyl, acetyl or a group 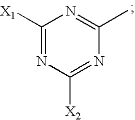
R₂ is C₂-C₆alkylene;
X₁-X₈ independently of one another are C₁-C₁₂alkyloxy, C₁-C₁₂alkylamino, C₁-C₁₂dialkylamino, carboxymethyl, halogen, —OH, NH₂, —SH, C₁-C₁₂alkyl H, C₁-C₁₂alkenylamino or C₁-C₁₂alkynylamino or groups of the formulas (d31)-(d34) 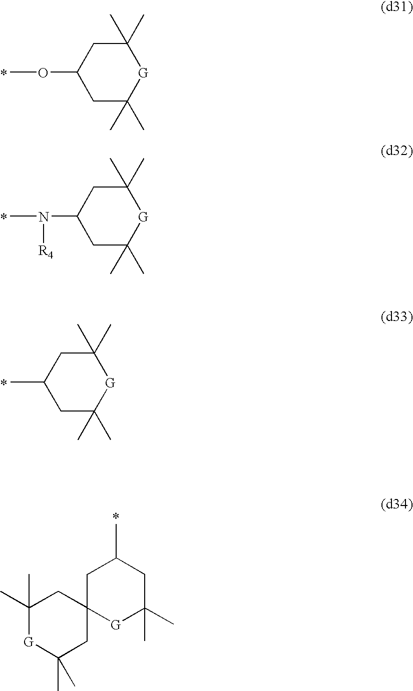
G is 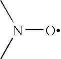
R₄ is hydrogen, C₁-C₄alkyl or a group of formula (d33); 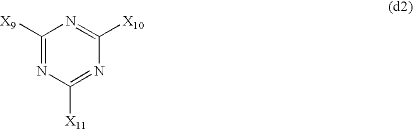
wherein X₉-X₁₁ independently of one another are as defined for X₁-X₈; 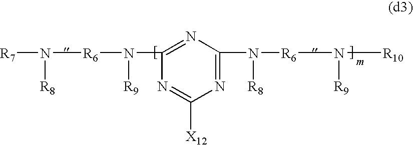
wherein
m is 0-50
R₆ is C₂-C₆alkylene or a direct bond;
R₇, R₈, R₉ and R₁₀ independently are H, methyl, formyl, acetyl or a group 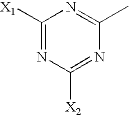
and X₁₂ has the meaning as defined for X₁-X₈; 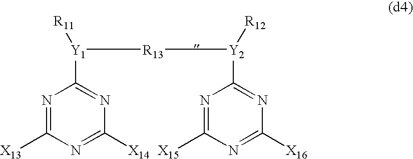
wherein
Y₁ and Y₂ are N, O or S
R₁₃ is C₂-C₆alkylene and when Y₁ and Y₂ are N or S R₁₃ may also be a direct bond;
If Y₁ or Y₂ are O or S, then R₁₁ or R₁₂ are an electron pair;
If Y₁ and Y₂ are N, then R₁₁ and R₁₂ independently are H, C₁-C₄alkyl or a group of formula (d33) or a group 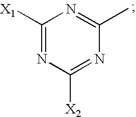
X₁₃-X₁₆ are independently as defined for X₁-X₈; 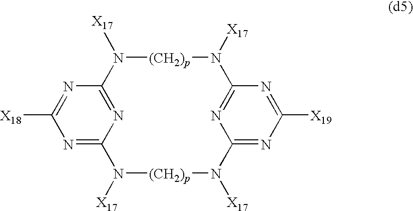
wherein
p is a number 2-6
X₁₇ is a group (d33); and
X₁₈ and X₁₉ are independently as defined for X₁-X₈; 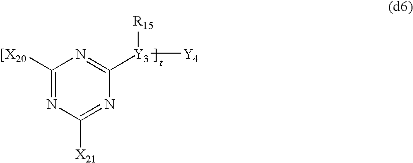
wherein
X₂₀ and X₂₁ are independently as defined for X₁-X₈;
Y₃ is N, O or S;
If Y₃ is O or S, then R₁₅ is an electron pair;
If Y₃ is N, then R₁₅ is H, C₁-C₄alkyl or a group of formula (d33);
t is 3;
Y₄ is a trivalent inorganic or organic residue; 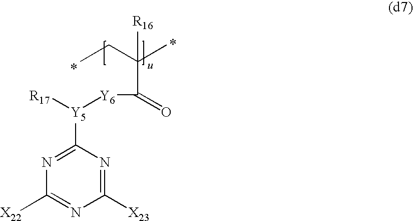
wherein
R₁₆ is H or CH₃;
Y₅ is N, O or S;
If Y₅ is O or S, then R₁₇ is an electron pair
If Y₅ is N, then R₁₇ is H or a group of formula (d33);
Y₆ is a direct bond, —NH— or —CH₂CH₂—N— or —CH₂CH₂—O—; and
X₂₂ and X₂₃ have the same meaning as defined for X₁-X₈.

11. A secondary battery according to claim 10 wherein the active material comprises a compound of formulae (e1) to (e7) 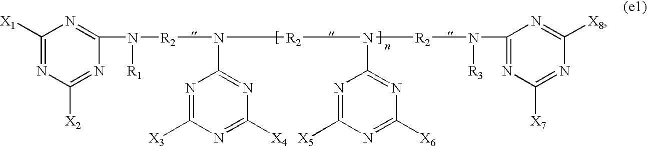
wherein
n is a number from 0-10
R₁ and R₃ are independently H or a group 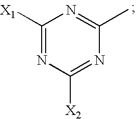
R₂ is —CH₂—CH₂—;
X₁—X₈ independently of one another are C₁-C₁₂alkylamino, C₁-C₁₂dialkylamino, halogen, NH₂, C₁-C₁₂alkenylamino or C₁-C₁₂alkynylamino or
groups of the formulas (e31)-(e34) 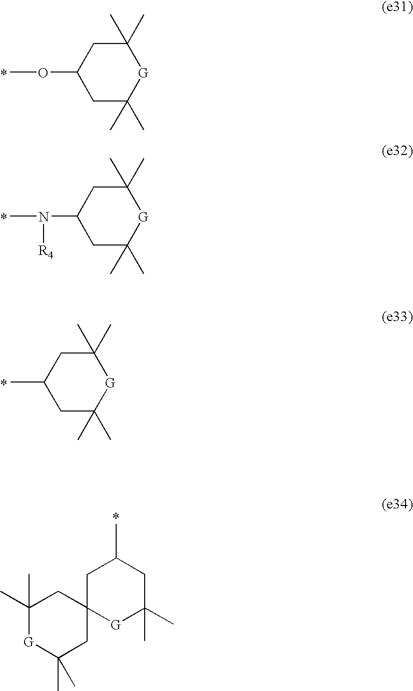
G is 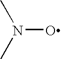
R₄ is hydrogen or a group of formula (e33); 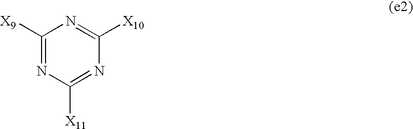
wherein X₉-X₁₁ independently of one another are as defined for X₁-X₈; 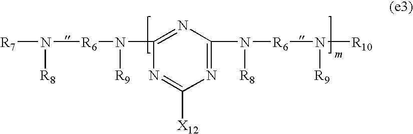
wherein
m is 0-10
R₆ is —CH₂CH₂— or a direct bond;
R₇, R₈, R₉ and R₁₀ are a group 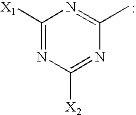
and X₁₂ has the meaning as defined for X₁-X₈; 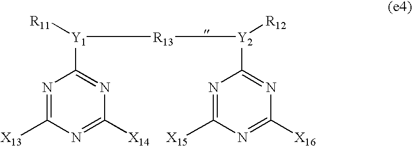
wherein
Y₁ and Y₂ are N, O or S
R₁₃ is —CH₂CH₂— and when Y₁ and Y₂ are N or S R₁₃ may also be a direct bond;
If Y₁ or Y₂ are O or S, then R₁₁ or R₁₂ are an electron pair;
If Y₁ and Y₂ are N, then R₁₁ and R₁₂ are a group of formula (e33) or a group 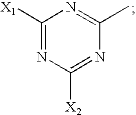
X₁₃-X₁₆ are independently as defined for X₁-X₈; 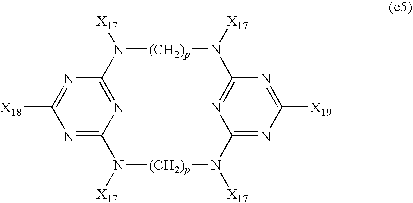
wherein
p is a number 2-6
X₁₇ is a group (e33); and
X₁₈ and X₁₉ are independently as defined for X₁-X₈; 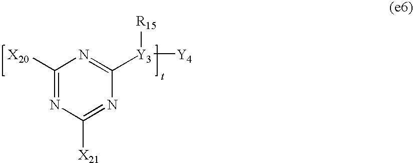
wherein
X₂₀ and X₂₁ are independently as defined for X₁-X₈;
Y₃ is N, O or S;
If Y₃ is O or S, then R₁₅ is an electron pair;
If Y₃ is N, then R₁₅ is H or a group of formula (e33);
t is 3;
Y₄ is a trivalent inorganic or organic residue; 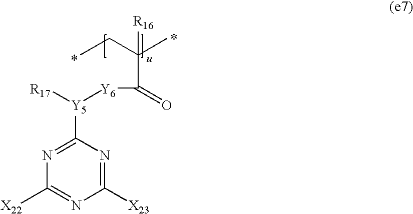
wherein
R₁₆ is H;
Y₅ is N or O;
If Y₅ is O, then R₁₇ is an electron pair
If Y₅ is N, then R₁₇ is H or a group of formula (e33);
Y₆ is a direct bond, —NH—, —CH₂CH₂—N— or —CH₂CH₂—O—; and
X₂₂ and X₂₃ have the same meaning as defined for X₁-X₈.

12. A method for providing a secondary battery, which method comprises incorporating into at least one of the positive or negative electrodes an active material comprising a compound selected from the group consisting of radicals of formulae (Ia) to (Iq), which are chemically bonded directly or via a linker group to a triazine structural element of formula (II) 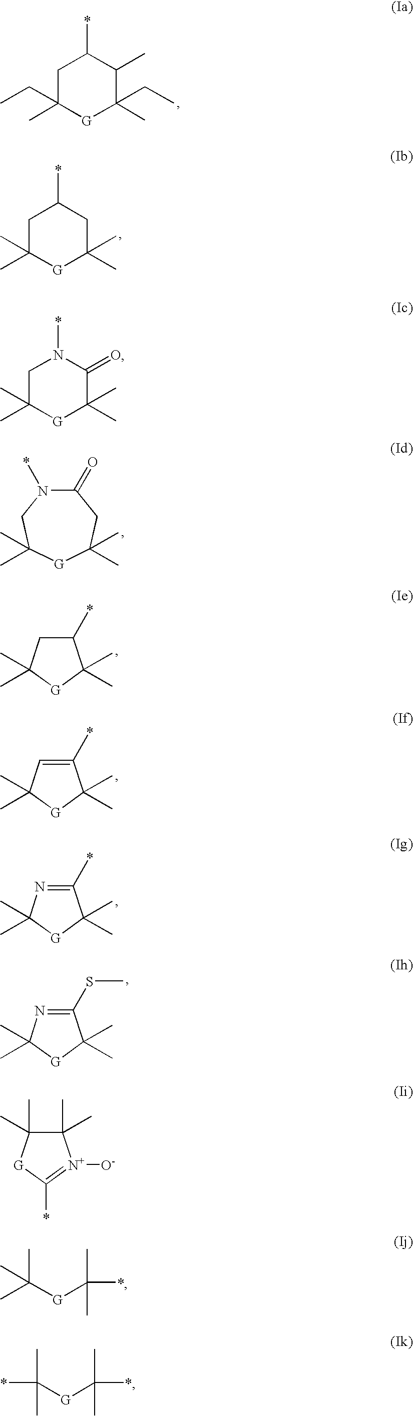 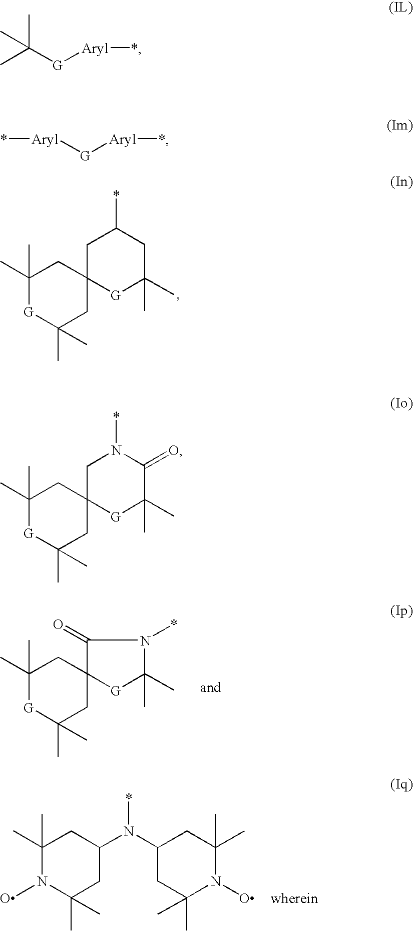
wherein
* is a valence indicating the bond to the triazine structural element;
Aryl is phenyl or naphthyl and
G is 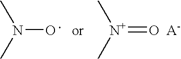
wherein A⁻ is an anion derived from an organic or inorganic acid; 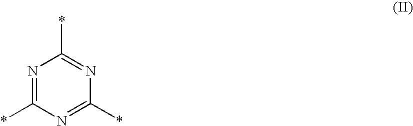
wherein
* indicates a bond to which at least one radical of formulae (Ia) to (Iq) is attached, either directly or via a linking group;
with the proviso that compound (A) is excluded

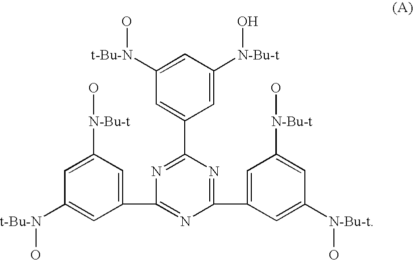

13. (canceled)

14. A compound of formulae (c1) to (c7) 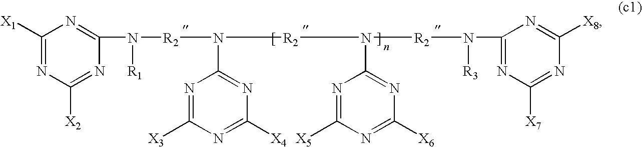
wherein
n is a number from 0-100;
R₁ and R₃ are independently H or C₁-C₁₂alkyl, C₅-C₇cycloalkyl, phenyl, C₁-C₁₂acyl or a group 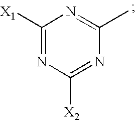
R₂ is C₂-C₁₂alkylene or phenylene;
X₁-X₈ independently of one another are C₁-C₁₂alkyloxy, C₅-C₆cycloalkyloxy, phenoxy, C₁-C₁₂acyloxy, C₁-C₁₂acylamino, C₁-C₁₂alkylamino, C₅-C₆cycloalkylamino, phenylamino, N-morpholino, C₁-C₁₂dialkylamino, C₅-C₆dicycloalkylamino, diphenylamino, C₁-C₁₂alkylthio, C₅-C₅cycloalkylthio, phenylthio, carboxymethyl, halogen, —OH, NH₂, —NH—NH₂, —SH, C₁-C₁₂alkyl, phenyl, H, C₁-C₁₂alkenylamino, C₁-C₁₂alkynylamino, —SCN, —NHNH—C₁-C₁₂Alkyl, —NHNH-Phenyl, —N₃, C₅-C₆cycloalkyl, C₂-C₁₂alkenyl, C₂-C₁₂alkynyl, wherein the said alkyls are unsubstituted or substituted by OR₄, NR₄R₅ or SR₄, or the said alkyls comprise at least two carbon atoms and are uninterrupted or interrupted by —O—, —S— or —NR₄— or the said alkyl comprises at least two carbon atoms and is both substituted by OR₄, —NR₄R₅ or SR₄ and interrupted by —O—, —S— or —NR₄—
or groups of the formulas (c31)-(c45) 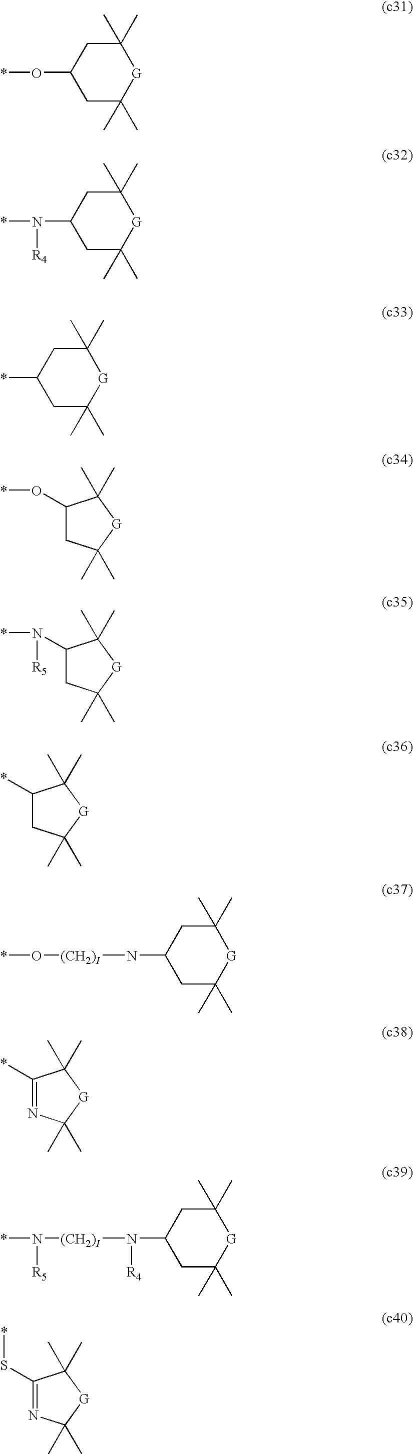 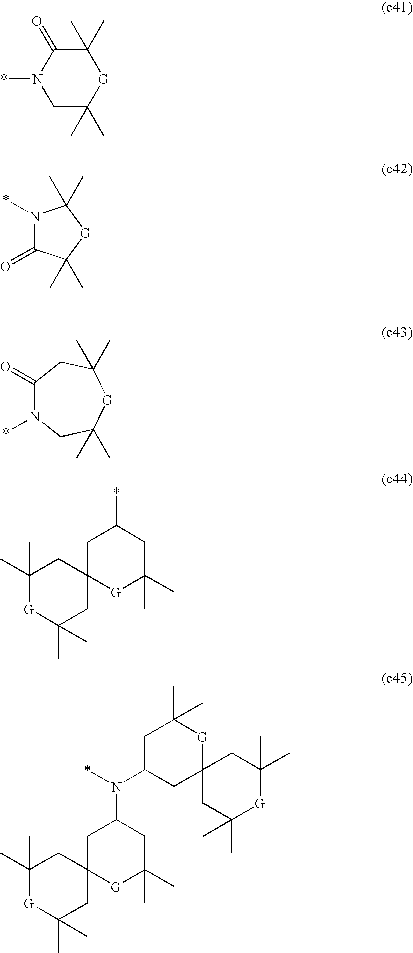
wherein
I is 2-6;
R₄ and R₅ are hydrogen, C₁-C₁₂alkyl, C₅-C₆cycloalkyl, phenyl, C₁-C₁₂acyl or a group of formula (c33) or (c36);
and
G is 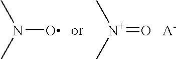
wherein A⁻ is an anion derived from an organic or inorganic acid;
with the proviso that at least one of X₁-X₈ is a group of formula 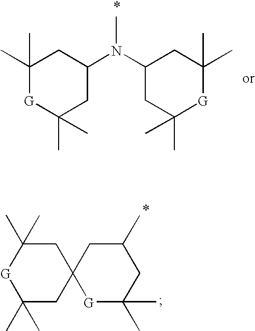 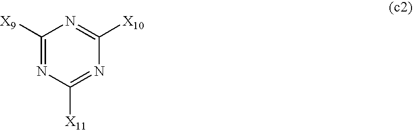
wherein X₉-X₁₁ independently of one another are groups as defined for X₁-X₈; 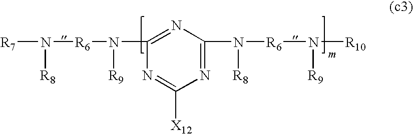
wherein
m is 0-100
R₆ is C₂-C₁₂alkylene, phenylene or a direct bond;
R₇, R₈, R₉ and R₁₀ independently are H or C₁-C₁₂alkyl, C₅-C₆cycloalkyl, phenyl, C₁-C₁₂acyl or the groups (c33) or (c36) or a group 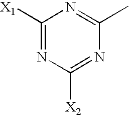
and X₁₂ has the meaning as defined for X₁-X₈; 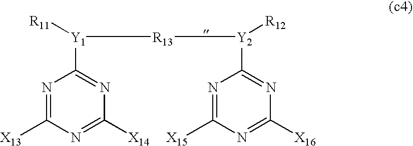
wherein
Y₁ and Y₂ are N, O or S
R₁₃ is C₂-C₁₂alkylene, phenylene and when Y₁ and Y₂ are N or S R₁₃ may also be a direct bond;
If Y₁ or Y₂ are O or S, then R₁₁ or R₁₂ are an electron pair;
If Y₁ and Y₂ are N, then R₁₁ and R₁₂ independently are H or C₁-C₁₂alkyl, C₅-C₆cycloalkyl, C₁-C₁₂acyl, phenyl or a group of formula (c33) or (c36) or a group 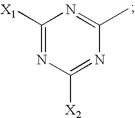
X₁₃-X₁₆ are independently as defined for X₁-X₈; 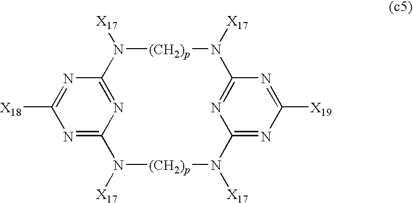
wherein
p is a number 0-18
X₁₇ is independently a group (c33) or (c36); and
X₁₈ and X₁₉ are independently groups as defined for X₁-X₈; 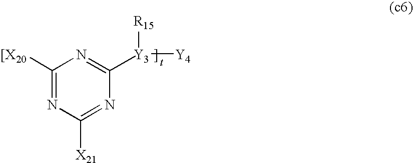
wherein
X₂₀ and X₂₁ are independently groups as defined for X₁-X₈;
Y₃ is N, O or S;
If Y₃ is O or S, then R₁₅ is an electron pair;
If Y₃ is N, then R₁₅ is H or C₁-C₁₂alkyl, C₅-C₆cycloalkyl, C₁-C₁₂acyl, phenyl or a group of formula (c33) or (c36) or a group 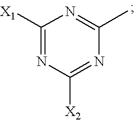
t is 3-6;
Y₄ is a trivalent-, tetravalent-, pentavalent or hexavalent inorganic or organic residue; 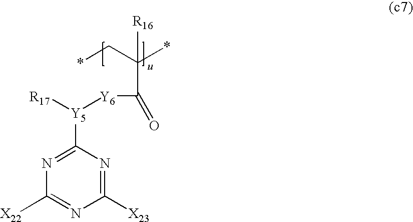
wherein
R₁₆ is H or CH₃;
Y₅ is N, O or S;
If Y₅ is O or S, then R₁₇ is an electron pair
If Y₅ is N, then R₁₇ is H or C₁-C₁₂alkyl, C₅-C₆cycloalkyl, C₁-C₁₂acyl, phenyl or a group of formula (c33) or (c36) or a group 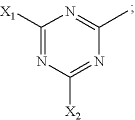
Y₆ is a direct bond, —NH— or —CH₂CH₂—N— or —CH₂CH₂—O—; and
X₂₂ and X₂₃ have the same meaning as defined for X₁-X₈.

15. A compound according to claim 14 of formulae (d1) to (d7) 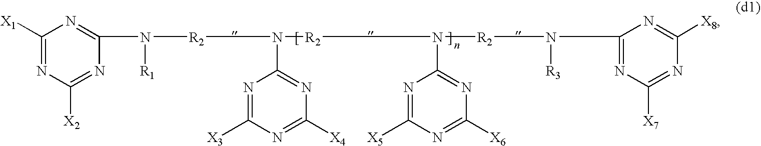
wherein
n is a number from 0-50
R₁ and R₃ are independently H, methyl, formyl, acetyl or a group 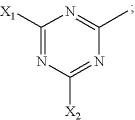
R₂ is C₂-C₆alkylene;
X₁-X₈ independently of one another are C₁-C₁₂alkyloxy, C₁-C₁₂alkylamino, C₁-C₁₂dialkylamino, carboxymethyl, halogen, —OH, NH₂, —SH, C₁-C₁₂alkyl H, C₁-C₁₂alkenylamino or C₁-C₁₂alkynylamino or
groups of the formulas (d31)-(d34), 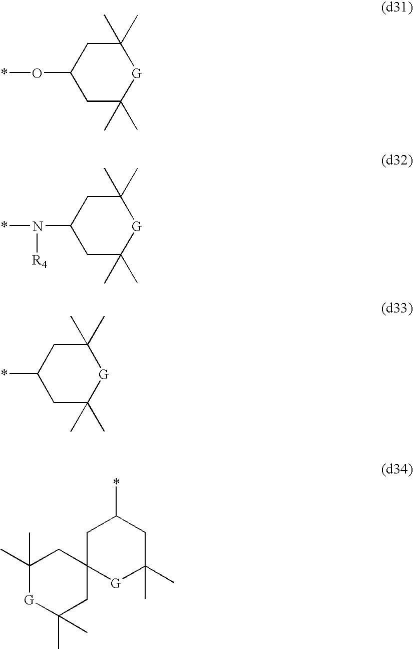
wherein G is 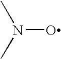
R₄ is hydrogen, C₁-C₄alkyl or a group of formula (d33)
with the proviso that at least one of X₁-X₈ is a group 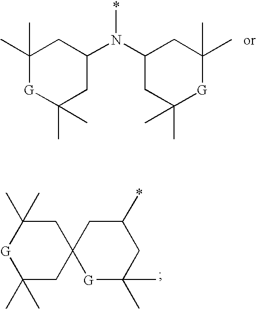
G is 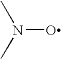
R₄ is hydrogen, C₁-C₄alkyl or a group of formula (d33); 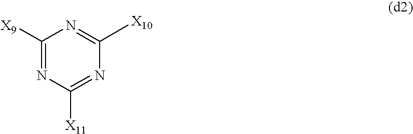
X₉-X₁₁ independently of one another are as defined for X₁-X₈; 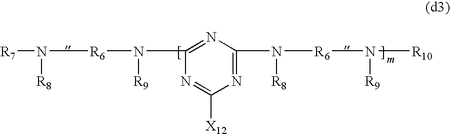
wherein
m is 0-50
R₆ is C₂-C₆alkylene or a direct bond;
R₇, R₈, R₉ and R₁₀ independently are H, methyl, formyl, acetyl or a group 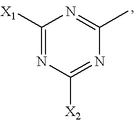
and X₁₂ has the meaning as defined for X₁-X₈; 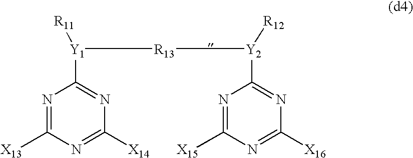
wherein
Y₁ and Y₂ are N, O or S
R₁₃ is C₂-C₆alkylene and when Y₁ and Y₂ are N or S R₁₃ may also be a direct bond;
If Y₁ or Y₂ are O or S, then R₁₁ or R₁₂ are an electron pair;
If Y₁ and Y₂ are N, then R₁₁ and R₁₂ independently are H, C₁-C₄alkyl or a group of formula (d33) or a group 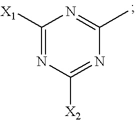
X₁₃-X₁₆ are independently as defined for X₁-X₈; 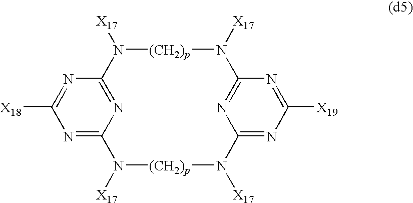
wherein
p is a number 2-6
X₁₇ is a group (d33); and
X₁₈ and X₁₉ are independently as defined for X₁-X₈; 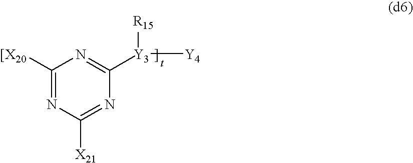
wherein
X₂₀ and X₂₁ are independently as defined for X₁-X₈;
Y₃ is N, O or S;
If Y₃ is O or S, then R₁₅ is an electron pair;
If Y₃ is N, then R₁₅ is H, C₁-C₄alkyl or a group of formula (d33);
t is 3;
Y₄ is a trivalent inorganic or organic residue; 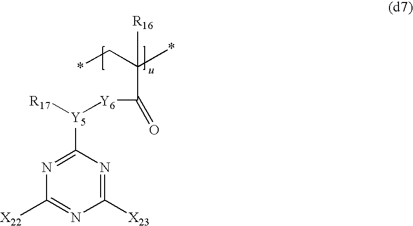
wherein
R₁₆ is H or CH₃;
Y₅ is N, O or S;
If Y₅ is O or S, then R₁₇ is an electron pair
If Y₅ is N, then R₁₇ is H or a group of formula (d33);
Y₆ is a direct bond, —NH— or —CH₂CH₂—N— or —CH₂CH₂—O—; and
X₂₂ and X₂₃ have the same meaning as defined for X₁-X₈.

16. A compound according to claim 15 of formulae (e1) to (e7) 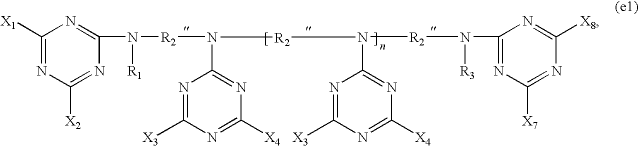
wherein
n is a number from 0-10
R₁ and R₃ are independently H or a group 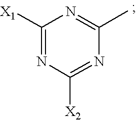
R₂ is —CH₂—CH₂—;
X₁-X₈ independently of one another are groups of the formulas (e31)-(e34) C₁-C₁₂alkylamino, C₁-C₁₂dialkylamino, halogen, NH₂, C₁-C₁₂alkenylamino or alkynylamino or groups of the formulas (e31)-(e34), 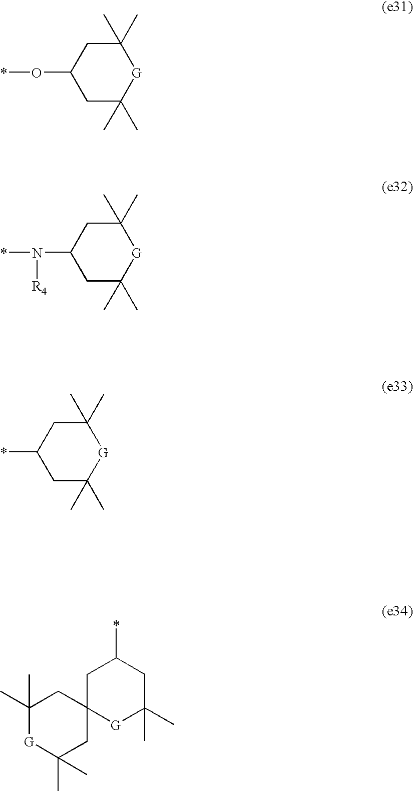
G is 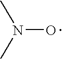
R₄ is hydrogen or a group of formula (e33)
with the proviso that at least one of X₁-X₈ is a group of formula 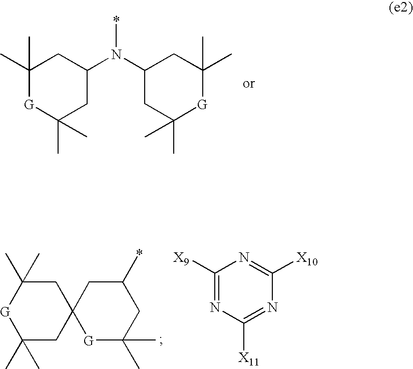
X₉-X₁₁ independently of one another are as defined for X₁-X₈; 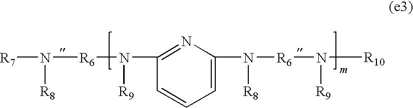
wherein
m is 0-10
R₆ is —CH₂CH₂— or a direct bond;
R₇, R₈, R₉ and R₁₀ are a group 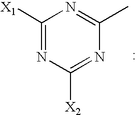
and X₁₂ has the meaning as defined for X₁-X₈; 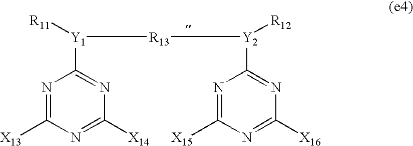
wherein
Y₁ and Y₂ are N, O or S
R₁₃ is —CH₂CH₂— and when Y₁ and Y₂ are N or S R₁₃ may also be a direct bond;
If Y₁ or Y₂ are O or S, then R₁₁ or R₁₂ are an electron pair;
If Y₁ and Y₂ are N, then R₁₁ and R₁₂ are a group of formula (e33) or a group 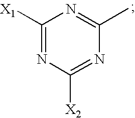
X₁₃-X₁₆ are independently as defined for X₁-X₈; 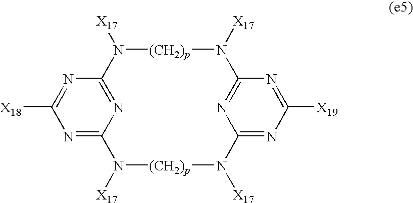
wherein
p is a number 2-6
X₁₇ is a group (e33); and
X₁₈ and X₁₉ are independently as defined for X₁-X₈; 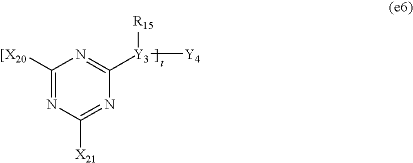
wherein
X₂₀ and X₂₁ are independently as defined for X₁-X₈,
Y₃ is N, O or S;
If Y₃ is O or S, then R₁₅ is an electron pair;
If Y₃ is N, then R₁₅ is H or a group of formula (e33);
t is 3;
Y₄ is a trivalent inorganic or organic residue; 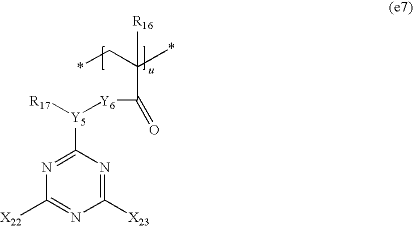
wherein
R₁₆ is H;
Y₅ is N or O;
If Y₅ is O, then R₁₇ is an electron pair
If Y₅ is N, then R₁₇ is H or a group of formula (e33);
Y₆ is a direct bond, —NH—, —CH₂CH₂—N— or —CH₂CH₂—O—; and
X₂₂ and X₂₃ have the same meaning as defined for X₁-X₈.
